SEQUENCE LISTING (103 sequences)

SEQ ID NO: 1 - protein, 1273 aa, SARS-COV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1255 aa, SARS-COV-2
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 3 - protein, 279 aa, SARS-COV-2
SNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGG

SEQ ID NO: 4 - protein, 283 aa, SARS-COV-2
SNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGEPEA

SEQ ID NO: 5 - protein, 279 aa, SARS-COV-2
SNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGG

SEQ ID NO: 6 - protein, 283 aa, SARS-COV-2
SNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGEPEA

SEQ ID NO: 7 - protein, 290 aa, SARS-COV-2
GIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITP

SEQ ID NO: 8 - protein, 278 aa, SARS-COV-2
SNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGG

SEQ ID NO: 9 - protein, 279 aa, SARS-COV-2
SNFRVQPTDSIVRFPNITNLCPFGEVFNATTFASVYAWNRKRISNCVADYSVLYNSTSFSTFKCYGVSPTKLNDLCFTNVYADSFVITGDEVRQIAPGQTGKIADYNYKLPDDFTGCVIAWNSKHIDAKEGGNFNYLYRLFRKANLKPFERDISTEIYQAGSKPCNGQTGLNCYYPLYRYGFYPTDGVGHQPYRVVVLSFELLNAPATVCGPKKSTNLVKNKCVNFNFNGLTGTGVLTESNKKFLPFQQFGRDIADTTDAVRDPQTLEILDITPCSFGG

SEQ ID NO: 10 - protein, 279 aa, SARS-COV-2
SNFRVQPTISIVRFPNITNLCPFGEVFNASKFASVYAWNRKRISNCVADYSVLYNSTSFSTFKCYGVSPTKLNDLCFTNVYADSFVVKGDEVRQIAPGQTGVIADYNYKLPDDFTGCVIAWNSVKQDALTGGNYGYLYRLFRKSKLKPFERDISTEIYQAGSTPCNGQVGLNCYYPLERYGFHPTTGVNYQPFRVVVLSXELLNGPATVCGPKLSTTLVKDKCVNFNFNGLTGTGVLTTSKKQFLPFQQFGRDISDTTDAVRDPQTLEILDITPCSFGG

SEQ ID NO: 11 - protein, 260 aa, SARS-COV-2
SNFRVSPTQEVVRFPNITNRCPFDKVFNATRFPSVYAWERTKISDCVADYTVLYNSTSFSTFKCYGVSPSKLIDLCFTSVYADTFLIRSSEVRQVAPGETGVIADYNYKLPDDFTGCVIAWNTAQQDKGQYYYRSSRKTKLKPFERDLSSDENGVRTLSTYDFYPTVPIEYQATRVVVLSFELLNAPATVCGPKLSTGLVKNQCVNFNFNGLKGTGVLTDSSKRFQSFQQFGRDTSDFTDSVRDPQTLQVLDITPCSFGG

SEQ ID NO: 12 - protein, 260 aa, SARS-COV-2
SNFRVSPTHEVIRFPNITNRCPFDKVFNASRFPNVYAWERTKISDCVADYTVLYNSTSFSTFKCYGVSPSKLIDLCFTSVYADTFLIRSSEVRQVAPGETGVIADYNYKLPDDFTGCVIAWNTAKQDQGQYYYRSSRKTKLKPFERDLSSDENGVRTLSTYDFYPTVPIEYQATRVVVLSFELLNAPATVCGPKLSTGLVKNQCVNFNFNGLKGTGVLTDSSKRFQSFQQFGRDTSDFTDSVRDPQTLQILDITPCSFGG

SEQ ID NO: 13 - protein, 260 aa, SARS-COV-2
SNFRVSPTHEVVRFPNITNRCPFDKVFNASRFPNVYAWERTKISDCVADYTVLYNSTSFSTFKCYGVSPSKLIDLCFTSVYADTFLIRSSEVRQVAPGETGVIADYNYKLPDDFTGCVIAWNTAKQDQGQYYYRSSRKTKLKPFERDLTSDENGVRTLSTYDFYPNVPIEYQATRVVVLSFELLNAPATVCGPKLSTALVKNQCVNFNFNGLKGIGVLTDSSKRFQSFQQFGRDTSDFTDSVRDPQTLQILDITPCSFGG

SEQ ID NO: 14 - protein, 260 aa, SARS-COV-2
SNFRVSPTHEVIRFPNITNRCPFDKVFNASRFPNVYAWERTKISDCVADYTVLYNSTSFSTFKCYGVSPSKLIDLCFTSVYADTFLIRSSEVRQVAPGETGVIADYNYKLPDDFTGCVIAWNTAKQDQGQYYYRSSRKTKLKPFERDLTSDENGVRTLSTYDFYPNVPIEYQATRVVVLSFELLNAPATVCGPKLSTGLVKNQCVNFNFNGLKGTGVLTDSSKRFQSFQQFGRDTSDFTDSVRDPQTLQILDITPCSFGG

SEQ ID NO: 15 - protein, 260 aa, SARS-COV-2
SNFRVSPTQEVIRFPNITNRCPFDKVFNASRFPNVYAWERTKISDCVADYTVLYNSTSFSTFKCYGVSPSKLIDLCFTSVYADTFLIRSSEVRQVAPGETGVIADYNYKLPDDFTGCVIAWNTAKQDQGQYYYRSSRKTKLKPFERDLSSDENGVRTLSTYDFYPTVPIEYQATRVVVLSFELLNAPATVCGPKLSTGLVKNQCVNFNFNGLKGTGVLTDSSKRFQSFQQFGRDMSDFTDSVRDPQTLQILDITPCSFGG

SEQ ID NO: 16 - protein, 260 aa, SARS-COV-2
SNFRVSPTQEVIRFPNITNRCPFDKVFNATRFPNVYAWERTKISDCVADYTVLYNSTSFSTFKCYGVSPSKLIDLCFTSVYADTFLIRSSEVRQVAPGETGVIADYNYKLPDDFTGCVIAWNTAKQDQGQYYYRSHRKTKLKPFERDLSSDENGVRTLSTYDFYPSVPVAYQATRVVVLSFELLNAPATVCGPKLSTQLVKNQCVNFNFNGLKGTGVLTESSKRFQSFQQFGRDTSDFTDSVRDPQTLEILDISPCSFGG

SEQ ID NO: 17 - protein, 260 aa, SARS-COV-2
SNFRVTPTQEVVRFPNITNRCPFDKVFNASRFPNVYAWERTKISDCVADYTVLYNSTSFSTFKCYGVSPSKLIDLCFTSVYADTFLIRSSEVRQVAPGETGVIADYNYKLPDDFTGCVIAWNTAQQDQGQYYYRSYRKEKLKPFERDLSSDENGVYTLSTYDFYPSIPVEYQATRVVVLSFELLNAPATVCGPKLSTQLVKNQCVNFNFNGLRGTGVLTTSSKRFQSFQQFGRDTSDFTDSVRDPQTLEILDISPCSFGG

SEQ ID NO: 18 - protein, 260 aa, SARS-COV-2; identical to SEQ ID NO: 17

SEQ ID NO: 19 - protein, 261 aa, SARS-COV-2
SNFRVSPTQEVIRFPNITNRCPFDKVFNATRFPNVYAWERTKISDCVADYTVLYNSTSFSTFKCYGVSPSKLIDLCFTSVYADTFLIRSSEVRQVAPGETGVIADYNYKLPDDFTGCVIAWNTAKHDTGNYYYRSHRKTKLKPFERDLSSDDGNGVYTLSTYDFNPNVPVAYQATRVVVLSFELLNAPATVCGPKLSTELVKNQCVNFNFNGLKGTGVLTSSSKRFQSFQQFGRDTSDFTDSVRDPQTLEILDISPCSFGG

SEQ ID NO: 20 - protein, 261 aa, SARS-COV-2
SNFRVSPTQEVIRFPNITNRCPFDKVFNVTRFPNVYAWERTKISDCVADYTVLYNSTSFSTFKCYGVSPSKLIDLCFTSVYADTFLIRSSEVRQVAPGETGVIADYNYKLPDDFTGCVIAWNTAKQDIGNYYYRSHRKTKLKPFERDLSSDDGNGVYTLSTYDFNPNVPVAYQATRVVVLSFELLNAPATVCGPKLSTQLVKNQCVNFNFNGLKGTGVLTSSSKRFQSFQQFGRDTSDFTDSVRDPQTLEILDISPCSFGG

SEQ ID NO: 21 - protein, 261 aa, SARS-COV-2
SNFRVSPTQEVIRFPNITNRCPFDKVFNASRFPNVYAWERTKISDCVADYTVLYNSTSFSTFKCYGVSPSKLIDLCFTSVYADTFLIRSSEVRQVAPGETGVIADYNYKLPDDFTGCVIAWNTAKQDTGHYYYRSHRKTKLKPFERDLSSDDGNGVYTLSTYDFNPNVPVAYQATRVVVLSFELLNAPATVCGPKLSTQLVKNQCVNFNFNGLKGTGVLTDSSKRFQSFQQFGRDTSDFTDSVRDPQTLEILDITPCSFGG

SEQ ID NO: 22 - protein, 261 aa, SARS-COV-2
SNFRVSPTQEVVRFPNITNRCPFDKVFNATRFPNVYAWERTKISDCVADYTVLYNSTSFSTFKCYGVSPSKLIDLCFTSVYADTFLIRSSEVRQVAPGETGVIADYNYKLPDDFTGCVIAWNTAKQDTGNYYYRSHRKTKLKPFERDLSSDDGNGVYTLSTYDFNPNVPVAYQATRVVVLSFELLNAPATVCGPKLSTQLVKNQCVNFNFNGLKGTGVLTPSLKRFQSFQQFGRDTSDFTDSVRDPQTLEILDISPCSFGG

SEQ ID NO: 23 - protein, 261 aa, SARS-COV-2
SNFRVSPTQEVIRFPNITNRCPFDRVFNASRFPSVYAWERTKISECVADYTVLYNSTSFSTFKCYGVSPSKLIDLCFTSVYADTFLIRSSEVRQVAPGETGVIADYNYKLPDDFTGCVIAWNTAKQDTGNYYYRSHRKTKLKPFERDLSSDDGNGVYTLSTYDFNPNVPVAYQATRVVVLSFELLNAPATVCGPKLSTQLVKNQCVNFNFNGLKGTGVLTPSSKRFQSFQQFGRDTSDFTDSVRDPQTLEILDISPCSFGG

SEQ ID NO: 24 - protein, 261 aa, SARS-COV-2
SNFRVTPTQEVVRFPNITNRCPFDRVFNASRFPSVYAWERTKISDCVADYTVLYNSTSFSTFKCYGVSPSKLIDLCFTSVYADTFLIRSSEVRQVAPGETGVIADYNYKLPDDFTGCVIAWNTAKQDTGYYYYRSHRKTKLKPFERDLSSDDGNGVYTLSTYDFNPNVPVAYQATRVVVLSFELLNAPATVCGPKLSTELVKNQCVNFNFNGLKGTGVLTKSSKRFQSFQQFGRDTSDFTDSVRDPQTLEILDISPCSFGG

SEQ ID NO: 25 - protein, 260 aa, SARS-COV-2
SNFRVSPTQEVVRFPNITNRCPFDKVFNATRFPSVYAWERTKISDCVADYTVLYNSTSFSTFKCYGVSPSKLIDLCFTSVYADTFLIRSSEVRQVAPGETGVIADYNYKLPDDFTGCVIAWNTANQDQGQYYYRSSRKEKLKPFERDLSSDENGVYTLSTYDFYPSVPLDYQATRVVVLSFELLNAPATVCGPKLSTTLVKNQCVNFNFNGLKGTGVLTASSKKFQSFQQFGRDASDFTDSVRDPQTLEILDISPCSFGG

SEQ ID NO: 26 - protein, 260 aa, SARS-COV-2
SNFRVSPSTEVIRFPNITNRCPFDRVFNASRFPSVYAWERTKISDCVADYTVLYNSTSFSTFKCYGVSPSKLIDLCFTSVYADTFLIRFSEVRQIAPGETGVIADYNYKLPDEFTGCVIAWNTANQDRGQYYYRSSRKTKLKPFERDLSSDENGVRTLSTYDFYPSVPLEYQATRVVVLSFELLNAPATVCGPKLSTSLIKNQCVNFNFNGLKGTGVLTDSSKKFQSFQQFGRDASDFTDSVRDPQTLQILDISPCSFGG

SEQ ID NO: 27 - protein, 260 aa, SARS-COV-2
SNFRVSPTREVVRFPNITNRCPFDSIFNASRFPSVYAWERTKISDCVADYTVLYNSTSFSTFKCYGVSPSKLIDLCFTSVYADTFLIRFSEVRQVAPGETGVIADYNYRLPDDFTGCVIAWNTANQDVGSYFYRSHRSTKLKPFERDLSSDENGVRTLSTYDFNPYVPLDYQATRVVVLSFELLNAPATVCGPKLSTELVKNQCVNFNFNGLKGTGVLSSSSKRFQSFQQFGRDASDFTDSVRDPQTLEILDITPCSFGG

SEQ ID NO: 28 - protein, 260 aa, SARS-COV-2
SNFRVSPTREVVRFPNITNRCPFDSIFNASRFPSVYAWERTKISDCVADYTVLYNSTLFSTFKCYGVSPSKLIDLCFTSVYADTFLIRFSEVRQVAPGETGVIADYNYRLPDDFTGCVIAWNTANQDVGSYFYRSHRSTKLKPFERDLSSDENGVRTLSTYDFNPNVPLDYQATRVVVLSFELLNAPATVCGPKLSTELVKNQCVNFNFNGLKGTGVLTSSSKRFQSFQQFGRDASDFTDSVRDPQTLEILDITPCSFGG

SEQ ID NO: 29 - protein, 260 aa, SARS-COV-2
SNFRVQPTVDVVRFPNITNLCPFDAVFNATRFPSVYAWERVKISNCVADYTAFYNSTSFSTFKCYGVSPSKLIDLCFTSVYADTFLIRFSEVRQVAPGETGVIADYNYKLPDDFTGCVIAWNTAKQDVGSYFYRSHRSSKLKPFERDLSSDENGVRTLSTYDFNPNVPLDYQATRVVVLSFELLNAPATVCGPKLSTQLVKNQCVNFNFNGLKGTGVLTDSSKRFQSFQQFGRDTSDFTDSVRDPQTLDILDITPCSFGG

SEQ ID NO: 30 - protein, 260 aa, SARS-COV-2
SNFRVSPVTEVVRFPNITNLCPFDKVFNATRFPSVYAWERTKISDCVADYTVFYNSTSFSTFNCYGVSPSKLIDLCFTSVYADTFLIRFSEVRQVAPGQTGVIADYNYKLPDDFTGCVIAWNTAKQDVGSYFYRSHRSSKLKPFERDLSSEENGVRTLSTYDFNQNVPLEYQATRVVVLSFELLNAPATVCGPKLSTSLVKNQCVNFNFNGFKGTGVLTDSSKTFQSFQQFGRDASDFTDSVRDPQTLRILDISPCSFGG

SEQ ID NO: 31 - protein, 260 aa, SARS-COV-2
SNFRVSPVTEVVRFPNITNLCPFDKVFNATRFPSVYAWERTKISDCVADYTVFYNSTSFSTFNCYGVSPSKLIDLCFTSVYADTFLIRFSEVRQVAPGQTGVIADYNYKLPDDFTGCVIAWNTAKQDVGSYFYRSHRSSKLKPFERDLSSEENGVRTLSTYDFNQYVPLEYQATRVVVLSFELLNAPATVCGPKLSTSLVKNQCVNFNFNGFKGTGVLTDSSKTFQSFQQFGRDASDFTDSVRDPQTLRILDISPCSFGG

SEQ ID NO: 32 - protein, 260 aa, SARS-COV-2
SNFRVSPVTEVVRFPNITNLCPFDKVFNATRFPSVYAWERTKISDCVADYTVFYNSTSFSTFNCYGVSPSKLIDLCFTSVYADTFLIRFSEVRQVAPGQTGVIADYNYKLPDDFTGCVIAWNTAKQDVGSYFYRSHRSSKLKPFERDLSSVEENGRTLSTYDFNQNVPLEYQATRVVVLSFELLNAPATVCGPKLSTSLVKNQCVNFNFNGFKGTGVLTDSSKTFQSFQQFGRDASDFTDSVRDPQTLRILDISPCSFGG

SEQ ID NO: 33 - protein, 260 aa, SARS-COV-2
SNFRVAPVTEVVRFPNITNLCPFDKVFNATRFPSVYAWERTKISDCVADYTVFYNSTSFSTFNCYGVSPSKLIDLCFTSVYADTFLIRFSEVRQVAPGQTGVIADYNYKLPDDFTGCVIAWNTAKYDVGSYFYRSHRSSKLKPFERDLSSEENGARTLSTYDFNQNVPLEYQATRVVVLSFELLNAPATVCGPKLSTSLVKNQCVNFNFNGFKGTGVLTDSSKTFQSFQQFGRDASDFTDSVRDPKTLQILDISPCSFGG

SEQ ID NO: 34 - protein, 260 aa, SARS-COV-2
SNFRVQPTVDVARFPNITNVCPFDKVFNATRFPSVYAWERTKISDCVADYTVFYNSTSFSTFNCYGVSPSKLIDLCFTSVYADTFLIRFSEVRQVAPGQTGVIADYNYKLPDDFIGCVIAWNTAKQDVGSYFYRSHRSSKLKPFERDLSSEENGVLTLSTYDFNQNVPLEYQATRVVVLSFELLNAPATVCGPKLSTPLVKNQCVNFNFNGLKGTGVLTDSSKTFQSFQQFGRDASDFTDSVRDPQTLQILDISPCSFGG

SEQ ID NO: 35 - protein, 261 aa, SARS-COV-2
SNFRVSPTQEVVRFPNITNRCPFDKVFNASRFPSVYAWERIKISDCVADYTVLYNSTSFSTFKCYGVSPSKLIDLCFTSVYADTFLIRSSEVRQVAPGETGVIADYNYKLPDDFTGCVIAWNTAKQDTGSYYYRSHRKTKLKPFERDLSSDDGNGVYTLSTYDFNPNVPVAYQATRVVVLSFELLNAPATVCGPKLSTQLVKNQCVNFNFNGLTGTGVLTPSSKRFQPFQQFGRDVSDFTDSVRDPKTSEILDISPCSFGG

SEQ ID NO: 36 - protein, 248 aa, SARS-COV-2
SRYRAQVAGFVRVTQRGSYCTPPYSVLQDPPQPVVWRRYMLYDCVFDFTVVVDSLPTHQLQCYGVSPRRLASMCYGSVTLDVMRINETHLNNLFNRVPDTFSLYNYALPDNFYGCLHAFYLNSTAPYAVANRFPIKPGGRQSNSAFIDTVINAAHYSPFSYVYGLAVITLKPAAGSKLVCPVANDTVVITDRCVQYNLYGYTGTGVLSKNTSLVIPDGKVFTASSTGTIIGVSINSTTYSIMPCVTVP

SEQ ID NO: 37 - protein, 364 aa, SARS-COV-2
NGYTVQPIADVYRRKPNLPNCNIEAWLNDKSVPSPLNWERKTFSNCNFNMSSLMSFIQADSFTCNNIDAAKIYGMCFSSITIDKFAIPNGRKVDLQLGNLGYLQSFNYRIDTTATSCQLYYNLPAANVSVSRFNPSTWNKRFGFIEDSVFKPRPAGVLTNHDVVYAQHCFKAPKNFCPCKLNGSCVGSGPGKNNGIGTCPAGTNYLTCDNLCTPDPITFTGTYKCPQTKSLVGIGEHCSGLAVKSDYCGGNSCTCRPQAFLGWSADSCLQGDKCNIFANFILHDVNSGLTCSTDLQKANTDIILGVCVNYDLYGILGQGIFVEVNATYYNSWQNLLYDSNGNLYGFRDYIINRTFMIRSCYSGR

SEQ ID NO: 38 - protein, 378 aa, SARS-COV-2
NGYTVQPIADVYRRIPNLPDCNIEAWLNDKSVPSPLNWERKTFSNCNFNMSSLMSFIQADSFTCNNIEAAKIYGMCFSSITIDKFAIPNGRKVDLQLGNLGYLQSFNYRIDTTAASCQLYYNLPAANVSVSRFNPSTWNRRFGFTEQSVFKPQPVGVFTHHDVVYAQHCFKAPTNFCPCKLDGSLCVGNGPGIDAGYKNSGIGTCPAGTNYLTCHNAAQCDCLCTPDPITSKSTGPYKCPQTKYLVGIGEHCSGLAIKSDYCGGNPCTCQPQAFLGWSVDSCLQGDRCNIFANFILHDVNSGTTCSTDLQKSNTDIILGVCVNYDLYGITGQGIFVEVNATYYNSWQNLLYDSNGNLYGFRDYLTNRTFMIRSCYSGR

SEQ ID NO: 39 - protein, 364 aa, SARS-COV-2
NGYTVQPVATVYRRIPDLPNCDIEAWLNSKTVSSPLNWERKIFSNCNFNMGRLMSFIQADSFGCNNIDASRLYGMCFGSITIDKFAIPNSRKVDLQVGKSGYLQSFNYKIDTAVSSCQLYYSLPAANVSVTHYNPSSWNRRYGFINQSFGSRGLHDAVYSQQCFNTPNTYCPCRTSQCIGGAGTGTCPVGTTVRKCFAAVTNATKCTCWCQPDPSTYKGVNAWTCPQSKVSIQPGQHCPGLGLVEDDCSGNPCTCKPQAFIGWSSETCLQNGRCNIFANFILNDVNSGTTCSTDLQQGNTNITTDVCVNYDLYGITGQGILIEVNATYYNSWQNLLYDSSGNLYGFRDYLSNRTFLIRSCYSGR

SEQ ID NO: 40 - protein, 368 aa, SARS-COV-2
SGYTVQPVGLVYRRVRNLPDCKIEEWLAANTVPSPLNWERKTFQNCNFNLSSLLRFVQAESLSCSNIDASKVYGMCFGSISIDKFAIPNSRRVDLQLGKSGLLQSFNYKIDTRATSCQLYYSLAQDNVTVINHNPSSWNRRYGFNDVATFHSGEHDVAYAEACFTVGASYCPCAKPSTVYSCVTGKPKSANCPTGTSNRECNVQASGFKSKCDCTCNPSPLTTYDPRCLQARSMLGVGDHCEGLGILEDKCGGSNICNCSADAFVGWAMDSCLSNARCHIFSNLMLNGINSGTTCSTDFQLPNTEVVTGVCVKYDLYGSTGQGVFKEVKADYYNSWQNLLYDVNGNLNGFRDIVTNKTYLLRSCYSGR

SEQ ID NO: 41 - protein, 372 aa, SARS-COV-2
SGFTVKPVATVHRRIPDLPDCDIDKWLNNFNVPSPLNWERKIFSNCNFNLSTLLRLVHTDSFSCNNFDESKIYGSCFKSIVLDKFAIPNSRRSDLQLGSSGFLQSSNYKIDTTSSSCQLYYSLPAINVTINNYNPSSWNRRYGFNNFNLSSHSVVYSRYCFSVNNTFCPCAKPSFASSCKSHKPPSASCPIGTNYRSCESTTVLDHTDWCRCSCLPDPITAYDPRSCSQKKSLVGVGEHCAGFGVDEEKCGVLDGSYNVSCLCSTDAFLGWSYDTCVSNNRCNIFSNFILNGINSGTTCSNDLLQPNTEVFTDVCVDYDLYGITGQGIFKEVSAVYYNSWQNLLYDSNGNIIGFKDFVTNKTYNIFPCYAGR

SEQ ID NO: 42 - protein, 370 aa, SARS-COV-2
SGFTVKPVATVYRRIPNLPDCDIDNWLNNVSVPSPLNWERRIFSNCNFNLSTLLRLVHVDSFSCNNLDKSKIFGSCFNSITVDKFAIPNRRRDDLQLGSSGFLQSSNYKIDISSSSCQLYYSLPLVNVTINNFNPSSWNRRYGFGSFNLSSYDVVYSDHCFSVNSDFCPCADPSVVNSCAKSKPPSAICPAGTKYRHCDLDTTLYVKNWCRCSCLPDPISTYSPNTCPQKKVVVGIGEHCPGLGINEEKCGTQLNHSSCFCSPDAFLGWSFDSCISNNRCNIFSNFIFNGINSGTTCSNDLLYSNTEISTGVCVNYDLYGITGQGIFKEVSAAYYNNWQNLLYDSNGNIIGFKDFLTNKTYTILPCYSGR

SEQ ID NO: 43 - protein, 329 aa, SARS-COV-2
SGYTVQPVGVVYRRVANLPACNIEEWLTARSVPSPLNWERKTFQNCNFNLSSLLRYVQAESLFCNNIDASKVYGRCFGSISVDKFAVPRSRQVDLQLGNSGFLQTANYKIDTAATSCQLHYTLPKNNVTINNHNPSSWNRRYGFNDAGVFGKNQHDVVYAQQCFTVRSSYCPCAQPDIVSPCTTQTKPKSAFVNVGDHCEGLGVLEDNCGNADPHKGCICANNSFIGWSHDTCLVNDRCQIFANILLNGINSGTTCSTDLQLPNTEVVTGICVKYDLYGITGQGVFKEVKADYYNSWQTLLYDVNGNLNGFRDLTTNKTYTIRSCYSGR

SEQ ID NO: 44 - protein, 240 aa, SARS-COV-2
SGYTVQPVGVVYRRVPNLPDCKIEEWLTAKSVPSPLNWERRTFQNCNFNLSSLLRYVQAESLSCNNIDASKVYGMCFGSVSVDKFAIPRSRQIDLQIGNSGFLQTANYKIDTAATSCQLYYSLPKNNVTINNYNPSSWNRRYGFKVNDRCQIFANILLNGINSGTTCSTDLQLPNTEVATGVCVRYDLYGITGQGVFKEVKADYYNSWQALLYDVNGNLNGFRDLTTNKTYTIRSCYSGR

SEQ ID NO: 45 - protein, 221 aa, SARS-COV-2
NFLDDNVLPETYVALPIYYQHTDINFTATASFGGSCYVCKPHQVNISLNGNTSVCVRTSHFSIRYIYNRVKSGSPGDSSWHIYLKSGTCPFSFSKLNNFQKFKTICFSTVEVPGSCNFPLEATWHYTSYTIVGALYVTWSEGNSITGVPYPVSGIREFSNLVLNNCTKYNIYDYVGTGIIRSSNQSLAGGITYVSNSGNLLGFKNVSTGNIFIVTPCNQPD

SEQ ID NO: 46 - protein, 223 aa, SARS-COV-2
SPIQSVELPVSIVSLPVYHKHTFIVLYVDFKPQSGGGKCFNCYPAGVNITLANFNETKGPLCVDTSHFTTKYVAVYANVGRWSASINTGNCPFSFGKVNNFVKFGSVCFSLKDIPGGCAMPIVANWAYSKYYTIGSLYVSWSDGDGITGVPQPVEGVSSFMNVTLDKCTKYNIYDVSGVGVIRVSNDTFLNGITYTSTSGNLLGFKDVTKGTIYSITPCNPPD

SEQ ID NO: 47 - protein, 285 aa, SARS-COV-2
CAGETNFKSLSLWDTPASDCVSGSYNQEATLGAFKVYFDLINCTFRYNYTITEDENAEWFGITQDTQGVHLYSSRKENVFRNNMFHFATLPVYQKILYYTVIPRSIRSPFNDRKAWAAFYIYKLHPLTYLLNFDVEGYITKAVDCGYDDLAQLQCSYESFEVETGVYSVSSFEASPRGEFIEQATTQECDFTPMLTGTPPPIYNFKRLVFTNCNYNLTKLLSLFQVSEFSCHQVSPSSLATGCYSSLTVDYFAYSTDMSSYLQPGSAGAIVQFNYKQDFSNPTCR

SEQ ID NO: 48 - protein, 295 aa, SARS-COV-2
SSYEASATGTFIEQPNATECDFSPMLTGVAPQVYNFKRLVFSNCNYNLTKLLSLFAVDEFSCNGISPDSIARGCYSTLTVDYFAYPLSMKSYIRPGSAGNIPLYNYKQSFANPTCRVMASVLANVTITKPHAYGYISKCSRLTGANQDVETPLYINPGEYSICRDFSPGGFSEDGQVFKRTLTQFEGGGLLIGVGTRVPMTDNLQMSFIISVQYGTGTDSVCPMLDLGDSLTITNRLGKCVDYSLYGVTGRGVFQNCTAVGVKQQRFVYDSFDNLVGYYSDDGNYYCVRPCVSVP

SEQ ID NO: 49 - protein, 295 aa, SARS-COV-2
SFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKY

SEQ ID NO: 50 - protein, 278 aa, SARS-COV-2
SNFRVSPSTEVVRFPNITNLCPFGQVFNASNFPSVYAWERLRISDCVADYAVLYNSSSSFSTFKCYGVSPTKLNDLCFSSVYADYFVVKGDDVRQIAPAQTGVIADYNYKLPDDFTGCVLAWNTNSVDSKSGNNFYYRLFRHGKIKPYERDISNVLYNSAGGTCSSISQLGCYEPLKSYGFTPTVGVGYQPYRVVVLSFELLNAPATVCGPKKSTELVKNKCVNFNFNGLTGTGVLTSSTKKFQPFQQFGRDVSDFTDSVRDPKTFEILDISPCSYGG

SEQ ID NO: 51 - protein, 275 aa, SARS-COV-2
SNFRVTPTTEVVRFPNITQLCPFNEVFNITSFPSVYAWERMRITNCVADYSVLYNSSASFSTFQCYGVSPTKLNDLCFSSVYADYFVVKGDDVRQIAPAQTGVIADYNYKLPDDFTGCVIAWNTNSLDSSNEFFYRRFRHGKIKPYGRDLSNVLFNPSGGTCSAEGLNCYKPLASYGFTQSSGIGFQPYRVVVLSFELLNAPATVCGPKQSTELVKNKCVNFNFNGLTGTGVLTNSTKKFQPFQQFGRDVSDFTDSVRDPKTLEILDIAPCSYGG

SEQ ID NO: 52 - protein, 278 aa, SARS-COV-2
SNFRVAPSKEVVRFPNITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFLGCVLAWNTNSKDSSTSGNYNYLYRWVRRSKLNPYERDLSNDIYSPGGQSCSAIGPNCYNPLRPYGFFTTAGVGHQPYRVVVLSFELLNAPATVCGPKLSTDLIKNQCVNFNFNGLTGTGVLTSSSKRFQPFQQFGRDVSDFTDSVRDPKTSEILDISPCSFGG

SEQ ID NO: 53 - protein, 278 aa, SARS-COV-2
SNFRVAPSKEVVRFPNITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFLGCVLAWNTNSKDSSTSGNYNYLYRWVRRSKLNPYERDLSNDIYSPGGQSCSAVGPNCYNPLRPYGFFTTAGVGHQPYRVVVLSFELLNAPATVCGPKLSTDLIKNQCVNFNFNGLTGTGVLTPSSKRFQPFQQFGRDVSDFTDSVRDPKTSEILDISPCSFGG

SEQ ID NO: 54 - protein, 278 aa, SARS-COV-2
SNFRVAPSKEVVRFPNITNLCPFGEVFNATTFPSVYAWERKRISNCVADYSILYNSTSFSTFKCYGVSATKLNDLCFSNVYADSFVVKGDDVRQIAPGQTGVIADYNYKLPDDFLGCVLAWNTNSKDSSTSGNYNYLYRWVRRSKLNPYERDLSNDIYSPGGQSCSAVGPNCYNPLRPYGFFTTAGVGHQPYRVVVLSFELLNAPATVCGPKLSTDLIKNQCVNFNFNGLTGTGVLTPSSKRFQPFQQFGRDVSDFTDSVRDPKTSEILDISPCSFGG

SEQ ID NO: 55 - protein, 278 aa, SARS-COV-2
SNFRVAPSKEVVRFPNITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFTGCVLAWNTRNIDATQTGNYNYKYRSLRHGKLRPFERDISNVPFSPDGKPCTPPAFNCYWPLNDYGFYITNGIGYQPYRVVVLSFELLNAPATVCGPKLSTDLIKNQCVNFNFNGLTGTGVLTPSSKRFQPFQQFGRDVSDFTDSVRDPKTSEILDISPCSFGG

SEQ ID NO: 56 - protein, 278 aa, SARS-COV-2; identical to SEQ ID NO: 55

SEQ ID NO: 57 - protein, 278 aa, SARS-COV-2; identical to SEQ ID NO: 55

SEQ ID NO: 58 - protein, 278 aa, SARS-COV-2
SNFRVSPSKEVVRFPNITNLCPFGEVFNATTFPSVYAWERKRISNCVADYSVLYNSTSFSTFKCYGVSAIKLNDLCFSNVYADSFVVKGDDVRQIAPGQTGVIADYNYKLPDDFMGCVLAWNTRNIDATSSGNFNYKYRSLRHGKLRPFERDISNVPFSPDGKPCTPPAFNCYWPLNDYGFYTTNGIGYQPYRVVVLSFELLNAPATVCGPKLSTDLITNQCVNFNFNGLTGTGVLTPSLKRFQPFQQFGRDFSDFTDSVRDPKTLEVLDISPCSFGG

SEQ ID NO: 59 - protein, 278 aa, SARS-COV-2
SNFRVSPSREVVRFPNITNLCPFGEVFNATTFPSVYAWERKRISNCVADYSVLYNSTSFSTFKCYGVSAIKLNDLCFSNVYADSFVVKGDDVRQIAPGQTGVIADYNYKLPDDFMGCVLAWNTRNIDATSSGNFHYKYRSLRHGKLRPFERDISNVPFSPDGKPCTPPAFNCYWPLNDYGFYTTNGIGYQPYRVVVLSFELLNAPATVCGPKLSTDLITNQCVNFNFNGLTGTGVLTPSSKRFQPFQQFGRDVSDFTDSVRDPKTLEVLDISPCSFGG

SEQ ID NO: 60 - protein, 278 aa, SARS-COV-2
SNFRVAPSKEVVRFPNITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFMGCVLAWNTRNIDATSTGNYNYKYRSLRHGKLRPFERDISNVPFSPDGKPCTPPAFNCYWPLNDYGFFTTNGIGYQPYRVVVLSFELLNAPATVCGPKLSTDLIKNQCVNFNFNGLTGTGVLTSSSKRFQPFQQFGRDVSDFTDSVRDPKTSEILDISPCSFGG

SEQ ID NO: 61 - protein, 278 aa, SARS-COV-2
SNFRVVPSRDVVRFPNITNLCPFGEVFNATKFPSVYAWERKRISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGG

SEQ ID NO: 62 - protein, 278 aa, SARS-COV-2
SNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRCLRHGKLRPFERDISNVPFSPDGKPCTPPAFNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGG

SEQ ID NO: 63 - protein, 278 aa, SARS-COV-2; identical to SEQ ID NO: 8

SEQ ID NO: 64 - protein, 278 aa, SARS-COV-2
SNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLKHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGG

SEQ ID NO: 65 - protein, 278 aa, SARS-COV-2
SNFRVVPSGDVVRFPNITNLCPFGEVFNATKFPSVYAWERKKISNCVADYSVLYNSTFFSTFKCYGVSATKLNDLCFSNVYVDSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGG

SEQ ID NO: 66 - protein, 278 aa, SARS-COV-2
SNFRVVPSGDVVRFPNITNLCPFGEVFNATKFPSVYAWERKKISNCVVDYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGG

SEQ ID NO: 67 - protein, 278 aa, SARS-COV-2
SNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNHNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGG

SEQ ID NO: 68 - protein, 278 aa, SARS-COV-2
SNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPNGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGG

SEQ ID NO: 69 - protein, 278 aa, SARS-COV-2
SNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPAPNCYWPLNDYGFYTTSGIGYQPYRVVVLSFELLNAPATVCGPKLSTDLIKNQCVNFNFNGLTGTGVLTPSSKRFQPFQQFGRDVSDFTDSVRDPKTSEILDISPCAFGG

SEQ ID NO: 70 - protein, 278 aa, SARS-COV-2
SNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPAPNCYWPLNGYGFYTTSGIGYQPYRVVVLSFELLNAPATVCGPKLSTDLIKNQCVNFNFNGLTGTGVLTPSSKRFQPFQQFGRDVSDFTDSVRDPKTSEILDISPCSFGG

SEQ ID NO: 71 - protein, 278 aa, SARS-COV-2
SNFRVVPSGDVVRFPNITNLCPFGEVFNATKFPSVYAWERKRISNCVADYSVLYNSTSFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPAPNCYWPLNGYGFYTTSGIGYQPYRVVVLSFELLNAPATVCGPKLSTDLIKNQCVNFNFNGLTGTGVLTPSSKRFQPFQQFGRDVSDFTDSVRDPKTSEILDISPCSFGG

SEQ ID NO: 72 - protein, 278 aa, SARS-COV-2
SNFRVVPSGDVVRFPNITNLCPFGEVFNATKFPSVYAWERKRISNCVADYSVLYNSTSFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPAPNCYWPLRGYGFYTTSGIGYQPYRVVVLSFELLNAPATVCGPKLSTDLIKNQCVNFNFNGLTGTGVLTPSSKRFQPFQQFGRDVSDFTDSVRDPKTSEILDISPCSFGG

SEQ ID NO: 73 - protein, 278 aa, SARS-COV-2; identical to SEQ ID NO: 72

SEQ ID NO: 74 - protein, 278 aa, SARS-COV-2
SNFRVVPSGDVVRFPNITNLCPFGEVFNATKFPSVYAWERKRISNCVADYSVLYNSTSFSTFKCYGVSATKLNDLCFSNVYADSFVVKGDDVRQIAPGQTGVIADYNYKLPDDFMGCVLAWNTRNIDATSTGNYNYKHRYLRHGKLRPFERDISNVPFSPDGKPCTPPAPNCYWPLRGYGFYTTSGIGYQPYRVVVLSFELLNAPATVCGPKLSTDLIKNQCVNFNFNGLTGTGVLTPSSKRFQPFQQFGRDVSDFTDSVRDPKTSEILDISPCSFGG

SEQ ID NO: 75 - protein, 278 aa, SARS-COV-2
SNFRVVPSGDVVRFPNITNLCPFGEVFNATKFPSVYAWERKRISNCVADYSVLYNSTSFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSSDGKPCTPPAPNCYWPLRGYGFYTTSGIGYQPYRVVVLSFELLNAPATVCGPKLSTDLIKNQCVNFNFNGLTGTGVLTPSSKRFQPFQQFGRDVSDFTDSVRDPKTSEILDISPCSFGG

SEQ ID NO: 76 - protein, 278 aa, SARS-COV-2
SNFRVVPSGDVVRFPNITNLCPFGEVFNATKFPSVYAWERKRISNCVADYSVLYNSTSFSTFKCYGVSATKLNDLCFSNVYADSFVVKGDDVRQIAPGQTGVIADYNYKLPDDFMGCVLAWNTRNIDATSTGNYNYKXRYLRHGKLRPFERDISNVPFSPXGKPCTPPAPNCYWPLRGYGFYTTSGIGYQPYRVVVLSFELLNAPATVCGPKLSTDLIKNQCVNFNFNGLTGTGVLTPSSKRFQPFQQFGRDVSDFTDSVRDPKTSEILDISPCSFGG

SEQ ID NO: 77 - protein, 279 aa, SARS-COV-2
SNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGPNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGG

SEQ ID NO: 78 - protein, 283 aa, SARS-COV-2
SNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGPNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGEPEA

SEQ ID NO: 79 - protein, 279 aa, SARS-COV-2
SNFRVQPTESIVRFPNITNLCPFHEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYLYRLFRKSKLKPFERDISTEIYQAGNKPCNGVAGFNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGG

SEQ ID NO: 80 - protein, 283 aa, SARS-COV-2
SNFRVQPTESIVRFPNITNLCPFHEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYLYRLFRKSKLKPFERDISTEIYQAGNKPCNGVAGFNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGEPEA

SEQ ID NO: 81 - protein, 279 aa, SARS-COV-2
SNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSTVGGNYNYRYRLFRKSKLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGG

SEQ ID NO: 82 - protein, 283 aa, SARS-COV-2
SNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSTVGGNYNYRYRLFRKSKLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGEPEA

SEQ ID NO: 83 - protein, 279 aa, SARS-COV-2
SNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNRPCNGVAGPNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGG

SEQ ID NO: 84 - protein, 283 aa, SARS-COV-2
SNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNRPCNGVAGPNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGEPEA

SEQ ID NO: 85 - protein, 279 aa, SARS-COV-2
SNFRVQPTESIVRFPNITNLCPFHEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSTVSGNYNYRYRLFRKSKLKPFERDISTEIYQAGNKPCNGVAGSNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGG

SEQ ID NO: 86 - protein, 283 aa, SARS-COV-2
SNFRVQPTESIVRFPNITNLCPFHEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSTVSGNYNYRYRLFRKSKLKPFERDISTEIYQAGNKPCNGVAGSNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGEPEA

SEQ ID NO: 87 - protein, 279 aa, SARS-COV-2
SNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGSNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGG

SEQ ID NO: 88 - protein, 283 aa, SARS-COV-2
SNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGSNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGEPEA

SEQ ID NO: 89 - protein, 279 aa, SARS-COV-2
SNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGPNCYSPLQSYGFRPTYGVGHQPYRVVVLSFELLHASATVCGPKKSTNLVKNKCVNFNFNGLTGTGVLTESNKKFLPFQQFGRDIADTTDAVRDPQTLEILDITPCSFGG

SEQ ID NO: 90 - protein, 283 aa, SARS-COV-2
SNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGPNCYSPLQSYGFRPTYGVGHQPYRVVVLSFELLHASATVCGPKKSTNLVKNKCVNFNFNGLTGTGVLTESNKKFLPFQQFGRDIADTTDAVRDPQTLEILDITPCSFGGEPEA

SEQ ID NO: 91 - protein, 279 aa, SARS-COV-2
SNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLLRKSKLKPFERDISTEIYQAGNKPCNGVAGPNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGG

SEQ ID NO: 92 - protein, 283 aa, SARS-COV-2
SNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLLRKSKLKPFERDISTEIYQAGNKPCNGVAGPNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGEPEA

SEQ ID NO: 93 - protein, 279 aa, SARS-COV-2
SNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRFLRKSKLKPFERDISTEIYQAGNKPCNGVAGPNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGG

SEQ ID NO: 94 - protein, 283 aa, SARS-COV-2
SNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRFLRKSKLKPFERDISTEIYQAGNKPCNGVAGPNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGEPEA

SEQ ID NO: 95 - protein, 279 aa, SARS-COV-2; identical to SEQ ID NO: 93

SEQ ID NO: 96 - protein, 283 aa, SARS-COV-2; identical to SEQ ID NO: 94

SEQ ID NO: 97 - protein, 278 aa, SARS-COV-2
SNFRVQPTESIVRFPNVTNLCPFHEVFNATRFASVYAWNRTRISNCVADYSVLYNFAPFFAFKCYGVSPTKLNDLCFTNVYADSFVIKGNEVSQIAPGQTGNIADYNYKLPDDFTGCVIAWNSNKLDSKHSGNYDYWYRLFRKSKLKPFERDISTEIYQAGNKPCKGKGPNCYFPLQSYGFRPTYGVGHQPYRVVVLSFELLHAPATVCGPKKSTNLVKNKCVNFNFNGLTGTGVLTKSNKKFLPFQQFGRDIVDTTDAVRDPQTLEILDITPCSFGG

SEQ ID NO: 98 - protein, 282 aa, SARS-COV-2
SNFRVQPTESIVRFPNVTNLCPFHEVFNATRFASVYAWNRTRISNCVADYSVLYNFAPFFAFKCYGVSPTKLNDLCFTNVYADSFVIKGNEVSQIAPGQTGNIADYNYKLPDDFTGCVIAWNSNKLDSKHSGNYDYWYRLFRKSKLKPFERDISTEIYQAGNKPCKGKGPNCYFPLQSYGFRPTYGVGHQPYRVVVLSFELLHAPATVCGPKKSTNLVKNKCVNFNFNGLTGTGVLTKSNKKFLPFQQFGRDIVDTTDAVRDPQTLEILDITPCSFGGEPEA

SEQ ID NO: 99 - protein, 278 aa, SARS-COV-2
SNFRVQPTESIVRFPNVTNLCPFHEVFNATRFASVYAWNRTRISNCVADYSVLYNFAPFFAFKCYGVSPTKLNDLCFTNVYADSFVIKGNEVSQIAPGQTGNIADYNYKLPDDFTGCVIAWNSNKLDSKHSGNYDYWYRSFRKSKLKPFERDISTEIYQAGNKPCKGKGPNCYFPLQSYGFRPTYGVGHQPYRVVVLSFELLHAPATVCGPKKSTNLVKNKCVNFNFNGLTGTGVLTKSNKKFLPFQQFGRDIVDTTDAVRDPQTLEILDITPCSFGG

SEQ ID NO: 100 - protein, 282 aa, SARS-COV-2
SNFRVQPTESIVRFPNVTNLCPFHEVFNATRFASVYAWNRTRISNCVADYSVLYNFAPFFAFKCYGVSPTKLNDLCFTNVYADSFVIKGNEVSQIAPGQTGNIADYNYKLPDDFTGCVIAWNSNKLDSKHSGNYDYWYRSFRKSKLKPFERDISTEIYQAGNKPCKGKGPNCYFPLQSYGFRPTYGVGHQPYRVVVLSFELLHAPATVCGPKKSTNLVKNKCVNFNFNGLTGTGVLTKSNKKFLPFQQFGRDIVDTTDAVRDPQTLEILDITPCSFGGEPEA

SEQ ID NO: 101 - DNA, 20 bp, synthetic construct
  source 1..20: mol_type = other DNA
gtgatgctgctcttgctttg

SEQ ID NO: 102 - DNA, 20 bp, synthetic construct
  source 1..20: mol_type = other DNA
gtgacagtttggccttgttg

SEQ ID NO: 103 - protein, 4 aa, synthetic construct
EPEA

(19) United States
(12) Patent Application Publication
(10) Pub. No.: US 2025/0345415 A1
(43) Pub. Date: Nov. 13, 2025

(54) SARS-CoV-2 Vaccine Constructs

(71) Applicant: Rutgers, the State University of New Jersey, New Brunswick, NJ (US)

(72) Inventors: Stephen Anderson, Princeton, NJ (US); Elliot Campbell, Somerset, NJ (US)

(73) Assignee: Rutgers, the State University of New Jersey, New Brunswick, NJ (US)

(21) Appl. No.: 19/277,129

(22) Filed: Jul. 22, 2025

Related U.S. Application Data
(63) Continuation of application No. PCT/US24/12464, filed on Jan. 22, 2024.
(60) Provisional application No. 63/481,154, filed on Jan. 23, 2023.

(51) Int. Cl. A61K 39/215; A61K 39/00; A61P 31/14; C07K 14/005; C07K 14/705; C07K 16/10; C12N 7/00

(52) U.S. Cl. CPC A61K 39/215; A61P 31/14; C07K 14/005; C07K 14/70507; C07K 16/1003; C12N 7/00; A61K 2039/545; A61K 2039/555; A61K 2039/55561; A61K 2039/575; C07K 2319/20; C07K 2319/85; C12N 2770/20022; C12N 2770/20034

(57) ABSTRACT

The present disclosure describes, inter alia, fusion polypeptides comprising a SARS-CoV-2 Spike polypeptide fragment comprising at least a portion of the N-terminal domain, domains CD1, RBM, and CD2, and at least a portion of CTD1, wherein the N- or C-terminus of the Spike polypeptide fragment is fused to a heterologous N- or C-terminal tag comprising at least two, at least three, or at least four amino acids, as well as polynucleotides and vectors expressing such fusion polypeptides, pharmaceutical compositions comprising the polypeptides or polynucleotides encoding them, host cells for their production, and methods of using such pharmaceutical compositions as vaccines or for generation of antibodies.

### CROSS-REFERENCE TO RELATED APPLICATIONS

This application is a continuation of International Application No. PCT/US2024/012464, filed Jan. 22, 2024, which claims the benefit of priority of U.S. Provisional Application No. 63/481,154, filed Jan. 23, 2023, both of which are incorporated by reference herein in their entirety for any purpose.

### SEQUENCE LISTING

The present application is filed with a Sequence Listing in XML format. The Sequence Listing is provided as a file entitled “01308-0004-00US.xml” created on Jul. 1, 2025, which is 118,516 bytes in size. The information in the electronic format of the sequence listing is incorporated herein by reference in its entirety.

### FIELD

The present disclosure relates to particular SARS-CoV-2 (COVID-19) vaccine constructs and methods of making and using such constructs.

### BACKGROUND

The first approved SARS-CoV-2 vaccines were remarkably effective against the ancestral strain, with numerous clinical trials demonstrating a vaccine effectiveness of over 90% (references 1, 2). However, waning immunity and the emergence of new variants, many of which possess some degree of immune escape (3, 4), has necessitated boosters and spurred the development of variant-specific and pan-coronavirus vaccines. Further, despite the availability of approved vaccines, accessibility has been problematic outside of the developed world, and hesitancy towards vaccines and new vaccine technologies has slowed vaccination rates everywhere. Finally, efficacious vaccines and strategies for members of the population who are immunocompromised remain a significant scientific and medical challenge.

The continued research and development of novel vaccines, adjuvants, and immunization strategies to combat these weaknesses remains a high priority (5, 6). The WHO Target Product Profiles for COVID-19 Vaccines was revised in April 2022 to reflect this need and describes several desired characteristics for the next generation of vaccine candidates. Notable among these are the durability of protection, broader protection against emerging variants, and ease of manufacture and distribution. No current vaccine meets all of these criteria. Booster doses have been shown to enable protection against some emerging variants, but with rapid waning of effectiveness and continued vaccine hesitancy (7, 8) it is not clear whether current booster administration paradigms will comprise a sustainable strategy, even with variant-specific modifications to current vaccines (9, 10).

Among the earliest vaccines approved in the US and EU were two mRNA vaccines from Pfizer/BioNTech (BNT162b2) and Moderna (mRNA-1273), and two viral vectored vaccines from Janssen/J&J (Ad26.COV2.S) and Oxford/AstraZeneca (ADZ1222). The mRNA vaccines BNT162b2 and mRNA-1273 elicit extremely high antibody titers (11), but studies have shown that the immunity fades relatively quickly (12), prompting many countries to recommend a third booster dose and, presently, even a fourth or fifth booster in some cases (13). Unfortunately, even with multiple boosts, protection against SARS-CoV-2 variants remains modest (14). Conversely, the viral-vectored vaccines Ad26.COV2.S and AZD1222 elicit lower initial antibody responses (15), but protection seems to be more durable as immunological readouts remain relatively constant over time (12, 16). Perhaps most unexpectedly, and in stark contrast to the waning immunity observed with the mRNA vaccines, both the magnitude and breadth of the immune response increase with time after vaccination with Ad26.COV2.S (17, 18). The mechanisms mediating this non-waning behavior are not clear, but it may be due to differences in the kinetics of antigen presentation. The mRNA vaccines have been shown to produce a large bolus of short-lived Spike protein (19), whereas the viral-vectored vaccines may provide more modest, yet sustained, levels of antigen over a longer period (20).

With respect to the choice of immunogen, most approved vaccines use the full-length SARS-CoV-2 spike protein as immunogen. However, vaccines using a receptor binding domain-based (RBD-based) SARS-CoV-2 fragment have been shown to elicit a higher fraction of neutralizing antibodies (nAbs) than vaccines based on the full-length Spike protein, likely due to the entire immune response being directed toward the RBD (21, 22). Nonetheless, existing RBD vaccine candidates have often suffered from relatively poor expression and/or reduced immunogenicity. Accordingly, improved vaccine constructs are needed to address the shortcomings of existing vaccines, for example, to address the goals of greater potency as well as durability of protection, broader protection against emerging variants, and ease of manufacture and distribution.

### SUMMARY OF THE INVENTION

The present disclosure describes, inter alia, fusion polypeptides comprising a SARS-CoV-2 Spike polypeptide fragment that may be used as vaccines against SARS-CoV-2 and to generate antibodies, for example. The fusion polypeptides, for example, encompass the receptor binding domain (RBD), and comprise at least a portion of the N-terminal domain, domains CD1, RBM, and CD2, and at least a portion of CTD1, wherein the N- or C-terminus of the Spike polypeptide fragment is fused to a heterologous N- or C-terminal tag comprising at least two, at least three, or at least four amino acids. The present disclosure also describes polynucleotides and vectors expressing such fusion polypeptides, pharmaceutical compositions comprising the polypeptides or polynucleotides encoding them, host cells for their production, and methods of using such pharmaceutical compositions as vaccines, optionally in combination with particular adjuvants, and methods of using them for generation of antibodies.

Previous efforts to design RBD constructs for SARS-CoV-2 vaccines have, at times, attempted to trim the domain down to the “minimal expressible unit” containing the receptor binding motif (RBM), either by inspection or based upon homology to constructs used for other coronavirus RBDs (27-33). These approaches often truncate a significant portion of the protein structure “context” surrounding the RBM. The inventors herein have observed, however, that, from a structural biology perspective, this truncation could negatively impact protein folding and stability.

Indeed, several such constructs have been designed with key glycosylation sites knocked out, disulfides removed, or stabilizing mutations made within the structure in order to rescue protein expression (27, 28, 30). However, the present inventors have noted that such changes may lead to an immunogen 3D structure that differs from the native protein conformation against which the immune response is directed, potentially impacting antigenicity and utility as a vaccine.

In contrast, this present disclosure describes a novel protein component vaccine candidate called MT-001, based on a fragment of the SARS-CoV-2 spike protein that encompasses the receptor binding domain (RBD), including at least a portion of the N-terminal domain, domains CD1, RBM, and CD2, and at least a portion of CTD1, wherein the N- or C-terminus of the Spike polypeptide fragment is fused to a heterologous N- or C-terminal tag comprising at least two, at least three, or at least four amino acids. Mice and hamsters immunized with a prime-boost regimen of MT-001 unexpectedly demonstrated extremely high anti-spike IgG titers, while remarkably, this humoral response did not appreciably wane for a period of up to 12 months following vaccination. Also unexpectedly, virus neutralization titers, including titers against variants such as Delta and Omicron BA.1, also remained high throughout this long period, without the requirement for subsequent boosting.

For example, this disclosure encompasses, inter alia, (1) a fusion polypeptide comprising a SARS-CoV-2 Spike polypeptide fragment comprising at least a portion of the N-terminal domain, domains CD1, RBM, and CD2, and at least a portion of CTD1, wherein the N- or C-terminus of the Spike polypeptide fragment is fused to a heterologous N- or C-terminal tag comprising at least two, at least three, or at least four amino acids; (2) a fusion polypeptide comprising a SARS-CoV-2 Spike polypeptide fragment comprising at least a portion of the N-terminal domain, domains CD1, RBM, and CD2, and at least a portion of each of NT and CTD1, wherein the N- or C-terminus of the Spike polypeptide fragment is fused to a heterologous N- or C-terminal tag comprising at least two, at least three, or at least four amino acids, wherein the N- and C-terminal residues of the Spike polypeptide fragment are comprised within an antiparallel beta-sheet; and (3) a fusion polypeptide comprising a Spike polypeptide fragment comprising the amino acid sequence of residues 316-594 of SEQ ID NO: 1 or comprising an amino acid sequence of a Spike polypeptide fragment that aligns with residues 316-594 of SEQ ID NO: 1, wherein the N- or C-terminus of the Spike polypeptide fragment is fused to a heterologous N- or C-terminal tag comprising at least two, at least three, or at least four amino acids. The disclosure also encompasses, inter alia, any one of the following: (a) a fusion polypeptide, wherein the Spike polypeptide fragment comprises an amino acid sequence at least 90%, at least 95%, at least 97%, at least 98%, or at least 99% identical to the amino acid sequence of SEQ ID NO: 5; (b) a fusion polypeptide comprising the amino acid sequence of SEQ ID NO: 6; (c) a fusion polypeptide consisting of the amino acid sequence of SEQ ID NO: 6; (d) a fusion polypeptide comprising an amino acid sequence at least 90%, at least 95%, at least 97%, at least 98%, or at least 99% identical to the amino acid sequence of SEQ ID NO: 6; (e) a fusion polypeptide, wherein the Spike polypeptide fragment comprises an amino acid sequence at least 90%, at least 95%, at least 97%, at least 98%, or at least 99% identical to the amino acid sequence of SEQ ID NO: 77; (f) a fusion polypeptide comprising the amino acid sequence of SEQ ID NO: 78; (g) a fusion polypeptide consisting of the amino acid sequence of SEQ ID NO: 78; (h) a fusion polypeptide comprising an amino acid sequence at least 90%, at least 95%, at least 97%, at least 98%, or at least 99% identical to the amino acid sequence of SEQ ID NO: 78; (i) a fusion polypeptide, wherein the Spike polypeptide fragment comprises an amino acid sequence at least 90%, at least 95%, at least 97%, at least 98%, or at least 99% identical to the amino acid sequence of any one of SEQ ID NOs: 7-76, 79, 81, 83, 85, 87, 89, 91, 93, 95, 97, or 99; ( ) a fusion polypeptide comprising the amino acid sequence of any one of SEQ ID NOs: 7-100; (k) a fusion polypeptide consisting of the amino acid sequence of any one of SEQ ID NOs: 7-76 followed at the C-terminus by the amino acid sequence EPEA (SEQ ID NO: 103), or consisting of the amino acid sequence of any one of SEQ ID NOs: 80, 82, 84, 86, 88, 90, 92, 94, 96, 98, or 100; or (1) a fusion polypeptide comprising an amino acid sequence at least 90%, at least 95%, at least 97%, at least 98%, or at least 99% identical to the amino acid sequence of any one of SEQ ID NOs: 7-100.

In some embodiments, any of the above Spike polypeptide fragments may comprise one or more of the following amino acid substitutions: (a) a substitution at amino acid position 365 (with reference to SEQ ID NO: 1), such as Y365L; (b) a substitution at amino acid position 511 (with reference to SEQ ID NO: 1), such as V511A; (c) a substitution at amino acid position 402 (with reference to SEQ ID NO: 1), such as I402V; and/or (d) substitutions at amino acid positions 519-521 (with reference to SEQ ID NO: 1) so as to engineer an N-X-T sequence at those amino acid positions, wherein X is any residue but proline.

In any of the above fusion polypeptides, the N- or C-terminus of the Spike polypeptide fragment may be fused to a heterologous N- or C-terminal tag comprising at least two, at least three, or at least four amino acids. In some embodiments, the heterologous N- or C-terminal tag of the fusion polypeptide comprises a C-terminal tag. In some embodiments, the C-terminal tag comprises the amino acid sequence Glu-Pro-Glu-Ala (EPEA (SEQ ID NO: 103)). In some embodiments, the C-terminal tag consists of the amino acid sequence Glu-Pro-Glu-Ala (EPEA (SEQ ID NO: 103)).

In some embodiments, the Spike polypeptide fragment of the fusion polypeptide comprises the amino acid sequence of SEQ ID NO: 3. In some embodiments, the Spike polypeptide fragment comprises an amino acid sequence at least 90%, at least 95%, at least 97%, at least 98%, or at least 99% identical to the amino acid sequence of SEQ ID NO: 3.

In some embodiments, the fusion polypeptide comprises the amino acid sequence of SEQ ID NO: 4. In some embodiments, the fusion polypeptide consists of the amino acid sequence of SEQ ID NO: 4. In some embodiments, the fusion polypeptide comprises an amino acid sequence at least 90%, at least 95%, at least 97%, at least 98%, or at least 99% identical to the amino acid sequence of SEQ ID NO: 4.

In some embodiments, the Spike polypeptide fragment of the fusion polypeptide comprises the amino acid sequence of SEQ ID NO: 5. In some embodiments, the Spike polypeptide fragment comprises an amino acid sequence at least 90%, at least 95%, at least 97%, at least 98%, or at least 99% identical to the amino acid sequence of SEQ ID NO: 5.

In some embodiments, the fusion polypeptide comprises the amino acid sequence of SEQ ID NO: 6. In some embodiments, the fusion polypeptide consists of the amino acid sequence of SEQ ID NO: 6. In some embodiments, the fusion polypeptide comprises an amino acid sequence at least 90%, at least 95%, at least 97%, at least 98%, or at least 99% identical to the amino acid sequence of SEQ ID NO: 6.

In some embodiments, the Spike polypeptide fragment of the fusion polypeptide comprises the amino acid sequence of SEQ ID NO: 77. In some embodiments, the Spike polypeptide fragment comprises an amino acid sequence at least 90%, at least 95%, at least 97%, at least 98%, or at least 99% identical to the amino acid sequence of SEQ ID NO: 77.

In some embodiments, the fusion polypeptide comprises the amino acid sequence of SEQ ID NO: 78. In some embodiments, the fusion polypeptide consists of the amino acid sequence of SEQ ID NO: 78. In some embodiments, the fusion polypeptide comprises an amino acid sequence at least 90%, at least 95%, at least 97%, at least 98%, or at least 99% identical to the amino acid sequence of SEQ ID NO: 78.

In some embodiments, the Spike polypeptide fragment of the fusion polypeptide comprises the amino acid sequence of any one of SEQ ID NOs: 8-76, 79, 81, 83, 85, 87, 89, 91, 93, 95, 97, or 99. In some embodiments, the Spike polypeptide fragment comprises an amino acid sequence at least 90%, at least 95%, at least 97%, at least 98%, or at least 99% identical to the amino acid sequence of any one or more of SEQ ID NOs: 8-76, 79, 81, 83, 85, 87, 89, 91, 93, 95, 97, or 99.

In some embodiments, the fusion polypeptide comprises the amino acid sequence of any one of SEQ ID NOs: 8-76 μlus an EPEA (SEQ ID NO: 103) tag at the C-terminus or any one of SEQ ID NOs: 80, 82, 84, 86, 88, 90, 92, 94, 96, 98, or 100. In some embodiments, the fusion polypeptide consists of the amino acid sequence of any one of SEQ ID NOs: 8-76 μlus an EPEA (SEQ ID NO: 103) tag at the C-terminus or any one of SEQ ID NOs: 80, 82, 84, 86, 88, 90, 92, 94, 96, 98, or 100. In some embodiments, the fusion polypeptide comprises an amino acid sequence at least 90%, at least 95%, at least 97%, at least 98%, or at least 99% identical to the amino acid sequence of any one or more of SEQ ID NOs: 8-76 μlus an EPEA (SEQ ID NO: 103) tag at the C-terminus or any one of SEQ ID NOs: 80, 82, 84, 86, 88, 90, 92, 94, 96, 98, or 100.

In some embodiments, the fusion polypeptide is expressed in a fungal cell, such as a yeast cell, or an animal cell, such as an insect cell, or a mammalian cell, such as an HEK293 cell or CHO cell.

Embodiments herein also comprise compositions comprising a fusion polypeptide comprising two or more of the polypeptides described above. Embodiments herein also comprise compositions comprising at least one fusion polypeptide comprising one or more of the following amino acid substitutions: (a) a substitution at amino acid position 365 (with reference to SEQ ID NO: 1), such as Y365L; (b) a substitution at amino acid position 511 (with reference to SEQ ID NO: 1), such as V511A; (c) a substitution at amino acid position 402 (with reference to SEQ ID NO: 1), such as I402V; and/or (d) substitutions at amino acid positions 519-521 (with reference to SEQ ID NO: 1) so as to engineer an N-X-T sequence at those amino acid positions, wherein X is any residue but proline; and optionally further comprising the unsubstituted starting fusion polypeptide from which such mutant polypeptide(s) were derived.

The disclosure also contemplates pharmaceutical compositions comprising Spike polypeptide fragments and/or fusion polypeptides herein. In some embodiments, a pharmaceutical composition comprises a Spike polypeptide fragment or fusion polypeptide as disclosed herein and at least one adjuvant. In some embodiments, the adjuvant of the pharmaceutical composition comprises an aluminum salt and/or a Toll-like receptor (TLR) agonist. In some embodiments, the TLR agonist of the pharmaceutical composition is a TLR3, TLR4, TLR7, TLR8, TLR7/8, or TLR9 agonist. In some embodiments, the TLR agonist is a TLR9 agonist. In some embodiments, the TLR9 agonist is a CpG di-nucleotide agonist.

In some embodiments, the pharmaceutical composition has at least one of the following properties: (a) is capable of being administered annually; (b) provokes an immune response in a subject that has a durability of at least 6 months and/or of at least 1 year; (c) provokes an antibody-mediated immune response that does not wane after 6 months and/or after 1 year following administration; and (d) anti-RBD IgG antibody titer in a blood sample from a subject administered the pharmaceutical composition does not significantly reduce after 6 months, and/or after 1 year following administration. In some such cases, these properties are observed when the adjuvant comprises an aluminum salt but does not comprise a TLR agonist or does not comprise a CpG di-nucleotide agonist.

In some embodiments, a polynucleotide molecule encodes a fusion polypeptide as disclosed herein. In some embodiments, the polynucleotide molecule is a viral vector.

In some embodiments, a host cell expresses a polynucleotide molecule or a vector as disclosed herein.

In some embodiments, a method of preparing a fusion polypeptide as disclosed herein comprises incubating a host cell as disclosed herein under conditions allowing for expression of the fusion polypeptide, and optionally isolating the fusion polypeptide expressed by the host cell.

In some embodiments, a method of vaccinating an individual comprises administering a fusion polypeptide or a pharmaceutical composition as disclosed herein to the individual. In some embodiments, the method comprises administering the fusion polypeptide or pharmaceutical composition in a single dose. In some embodiments, the method comprises administering the fusion polypeptide or pharmaceutical composition in two doses within a two- to eight-week period of time. In some embodiments, the fusion polypeptide or pharmaceutical composition is administered to the individual every 6 months, every 9 months, or annually. In some embodiments, the method comprises administering concurrently or sequentially at least one adjuvant. In some embodiments, the at least one adjuvant comprises an aluminum salt such as aluminum hydroxide and/or a Toll-like receptor (TLR) agonist. In some embodiments, the TLR agonist is a TLR3, TLR4, TLR7, TLR8, TLR7/8, or TLR9 agonist. In some embodiments, the TLR agonist is a TLR9 agonist. In some embodiments, the TLR9 agonist is a CpG di-nucleotide agonist. In some cases, the at least one adjuvant comprises an aluminum salt such as aluminum hydroxide but does not comprise a TLR agonist. In some cases, the at least one adjuvant comprises an aluminum salt such as aluminum hydroxide but does not comprise a TLR9 agonist. In some cases, the at least one adjuvant comprises an aluminum salt such as aluminum hydroxide but does not comprise a CpG di-nucleotide agonist.

In some embodiments, administration of the fusion protein (a) provokes an immune response in a subject that has a durability of at least 6 months and/or of at least 1 year; (b) provokes an antibody-mediated immune response that does not wane after 6 months and/or after 1 year following administration; and/or (c) provokes an anti-RBD IgG antibody titer in a blood sample from the subject that does not significantly reduce after 6 months, and/or after 1 year following administration. For example, in some embodiments, these properties are observed when the fusion protein is administered with an aluminum salt agonist alone.

In some embodiments, a method of obtaining antibodies against a SARS-CoV-2 Spike polypeptide comprises administering a fusion polypeptide, a pharmaceutical composition, or a polynucleotide as disclosed herein to an animal, and optionally isolating antibodies produced by the animal or isolating B cells produced by the animal from which nucleic acids encoding specific antibodies can be cloned. In some embodiments, the antibodies and/or B cells produced by the animal are isolated 2 months, 3 months, 6 months, 9 months, or 12 months following administration of the fusion polypeptide, pharmaceutical composition, or polynucleotide to the animal.

In some embodiments, an isolated antibody is produced by a method disclosed herein.

The foregoing Summary and the further description that follows provide a non-limiting illustration of certain aspects of the disclosure.

### BRIEF DESCRIPTION OF THE DRAWINGS

FIGS. 1A-1C show detail of the SARS-CoV-2 spike protein in the region 300-600 and MT-001 construct design. FIG. 1A shows a structure of MT-001 construct derived from PDB IDs 7BYR and 7KNE. The RBD construct is organized by annotated blocks of amino acid sequence (“regions”, see panel C and ref. 45). Cysteines are shown as balls. The ligand of the RBM, ACE2 (from 7KNE), is shown as a gray molecular surface (left side of FIG. 1A). FIG. 1B shows the MT-001 construct (ribbon) shown in the context of the full-length spike trimer (space-filling model). FIG. 1C shows a schematic of the regions shown in FIG. 1A. Top of FIG. 1C: Region key for the MT-001 construct in FIG. 1A. NT: N-terminal region (residues 316-332); CD1: “core domain 1” region (333-436); RBM: receptor binding motif (437-508); CD2: “core domain 2” region (509-527); CTD1: “C-terminal domain 1” region (528-594). The 538-590 disulfide bond that stabilizes CTD1 is indicated by an arrow in FIG. 1A. Middle of FIG. 1C: % Ident. bars —Sequence identity per residue between SARS-CoV-2 spike and representative members of the coronavirus superfamily, demonstrating highly conserved regions N- and C-terminal to the RBM (FIG. 11). Variants bars—Sites of and frequency of mutations in characterized SARS-CoV-2 variants (3). Bottom of FIG. 1C: Schematic showing secondary structure and post-translational modifications in the region from residues 300-600 in the SARS-CoV-2 spike protein. Alpha helices are shown as cylinders, beta sheets as arrows, and turns as solid, black loops. Disulfide bonds are denoted with checkered, line bridges, and N-linked glycosylation sites with cross hatch circles. Bottom: Alignment of the MT-001 construct with the visualized region.

FIGS. 2A-2B show dose response and durability of anti-RBD serum IgG levels in mice vaccinated intramuscularly with Alhydrogel®-adjuvanted MT-001. FIG. 2A shows an immunization and bleeding schedule. Prime and secondary immunizations of the animals were at weeks 0 and 3, respectively; bleeds were performed on week 5, week 29, and week 52 to provide sera for analyses. Primary and secondary immunizations were with same amount of MT-001 antigen per animal—1 μg, 3 μg, or 15 μg—for each group of 10 mice. FIG. 2B shows midpoint (EC50) geometric mean anti-RBD IgG ELISA titers for each dosage group at each timepoint. GMTs are indicated numerically below each cluster of points.

FIGS. 3A-3E show immunization of mice with 3 μg of MT-001 antigen in Alhydrogel® with or without the TLR-9 agonist co-adjuvant, CpG ODN1826. FIG. 3A shows a schematic illustrating the MT-001 prime-boost regimen and bleeding schedule for 8-10-week-old female BALB/cJ mice (N=10). FIG. 3B shows RBD-specific IgG binding titers were assessed in mice immunized with 3 μg MT-001 adjuvanted with 500 μg Alhydrogel® only (−, open circles) or with 500 μg Alhydrogel® plus 20 μg CpG ODN1826 (+, closed circles). Binding antibody responses are displayed at 5, 15, 29, and 47 weeks post-primary immunization. The balanced Th1/Th2 response resulting from addition of CpG ODN1826 is evidenced by increased RBD-specific IgG1 (FIG. 3C) and IgG2 (FIG. 3D) antibody titers. This corresponded to an increased ratio of IgG2 to IgG1 antibody levels in CpG ODN1826-adjuvanted animals (FIG. 3E). Each circle (open or solid) represents the half-maximal titer for each serum sample averaged across at least three independent ELISAs. Horizontal bars indicate geometric mean titers per dose. P values reflect unpaired t tests between groups (*P<0.05; **P<0.01; ***P<0.001; ****P<0.0001).

FIGS. 4A-4E show SARS-CoV-2 challenge of hamsters vaccinated with MT-001 adjuvanted with Alhydrogel® and CpG ODN1826. FIG. 4A shows an MT-001 prime-boost regimen and a SARS-CoV-2 challenge schedule for Syrian golden hamsters. FIG. 4B shows midpoint hamster RBD-specific IgG ELISA GMTs. FIG. 4C shows lung viral load in hamsters vaccinated with 10 μg of MT-001 co-adjuvanted with 500 μg Alhydrogel®+100 μg CpG ODN1826, or mock-vaccinated with adjuvants alone, and infected with 10⁵ PFU of SARS-CoV-2 six weeks after the primary immunization (top). Individual data points and mean+/−S.D for MT-001 with adjuvants (open circles; n=8) or adjuvants alone (open squares; n=6) is shown. FIG. 4D shows viral RNA copy numbers in the lungs of hamsters vaccinated with MT-001 or adjuvants alone four days after intranasal infection with SARS-CoV-2. Data was analyzed by non-parametric Mann-Whitney test (**P<0.01). FIG. 4E shows representative plates from lung viral load assessment. Lung homogenates from adjuvant only (control) or MT-001 immunized (MT-001) hamsters were prepared 4 days post infection and used to infect Vero E6 cells. No plaques are observed with the lung homogenates from MT-001 immunized hamsters even at a 1:10 dilution, while plaques are visible from the lungs of control animals even at a 1:1,000,000 dilution.

FIGS. 5A-5B show SARS-CoV-2 variant neutralization. BALB/cJ mice (N=10 per group) were immunized twice at a three-week interval with 3 μg MT-001 and 500 μg Alhydrogel®, with or without 20 μg ODN1826 (CpG), as indicated. Mice were bled at 30 weeks post-primary immunization, and sera were assayed for antibody binding and neutralization. FIG. 5A shows anti-RBD midpoint ELISA titers were determined using the Wu-1 (WT) RBD or the Delta variant RBD (Delta) as a target. FIG. 5B shows virus neutralization titers at six months and eleven months post-primary immunization for mice immunized without (−CpG) or with (+CpG) were determined using SARS-CoV-2 USA-WA1/2020 (US/WA-1) or SARS-CoV-2 isolate hCoV-19/USA/MD-HP20874/2021 (Omicron BA. 1) as described in Methods. Geometric mean titers are as indicated. Asterisks indicate statistical significance as determined by a two-tailed Kruskal-Wallis test with subsequent Dunn's multiple-comparisons test (*P<0.05; **P<0.01).

FIG. 6 shows an exemplary DisMeta Output for the SARS-CoV-2 Spike protein from residues 300-600. Residues 300-600 correspond to numbers 1-301 in the plots. Secondary structure and detailed disordered region prediction results are shown.

FIGS. 7A-7B show expression (7A) and purification (7B) of MT-001. MT-001 was transiently expressed in 1L HEK293 suspension culture and purified in a single affinity chromatography step using CaptureSelect™ C-tagXL resin. Purified MT-001 was buffer exchanged into PBS containing 10% glycerol, and protein quantity, concentration, and purity were determined by UV-VIS spectroscopy and capillary electrophoresis, and oligomeric state was determined by HPLC-SEC. The purified protein was found to be predominantly monomeric (estimated size 33.78 kDa), with an apparent molecular weight by capillary electrophoresis under reducing conditions of 39.4 kDa (calculated 31.6 kDa) consistent with a glycosylated protein. The final purified yield of MT-001 was 160.49 mg.

FIGS. 8A-8C show hamster challenge data. Hamsters were immunized twice at a three-week interval with 10 μg MT-001, 500 μg Alhydrogel®, and 100 μg ODN1826 or a mock control where MT-001 was replaced with PBS. Six-weeks post-primary immunization, hamsters were challenged intranasally with 10⁵ PFU of SARS-CoV-2 US/WA-1. Body weight was monitored daily for four days, at which point the animals were sacrificed in order to collect sera and tissue samples for analysis.

FIG. 9 shows validation and correlation of anti-RBD IgG titers (using sandwich ELISA, FIG. 10) with anti-spike IgG titers (using standard ELISA, independently determined by a third party) obtained from the same samples. To determine if the MT-001 vaccine elicited a strong antibody response to RBD in the context of the entire SARS-CoV-2 spike protein and to validate our results, these sera were also independently analyzed by a third party (91). The resulting anti-spike IgG titers were similarly robust with significantly increased antibody levels (P=0.0028) in mice immunized with 15 μg MT-001 when compared to mice immunized with 1 g. For all sera tested, the ELISA results for our in-house indirect RBD binding assay were highly correlated with the independently performed anti-spike IgG binding assays

FIG. 10 shows a sandwich ELISA displaying 3D-conformational epitopes.

FIG. 11 shows a multiple sequence alignment of SARS-CoV-2 spike residues 300-600 with other members of the CoV family. Representative sequences were aligned using PROMALS3D (prodata.swmed.edu/promals3d) utilizing available structural information to ensure accurate domain alignments.

FIGS. 12A-12B show representative flow cytometry experiments (FIG. 12A) and an overall bar plot of RBD++ B cells in control mice (OVA), mice receiving MT-001, and mice receiving mixture composition MAV-004 (FIG. 12B), showing that the RBD-specific B cell response was significantly greater in MAV-004 compared to MT-001 and the control. The number of mice in each group is shown at the bottom of each bar plot in FIG. 12B and statistical significance is shown by P-values above the bar plot, comparing MAV-004 to the control and to MT-001.

### DETAILED DESCRIPTION OF CERTAIN EMBODIMENTS

### Definitions

Unless defined otherwise, all technical and scientific terms used herein have the same meaning as commonly understood by those of ordinary skill in the art, such as in the arts of peptide chemistry, cell culture and phage display, nucleic acid chemistry and biochemistry. Standard techniques are used for molecular biology, genetic and biochemical methods (see Sambrook et al., Molecular Cloning: A Laboratory Manual, 3rd ed., 2001, Cold Spring Harbor Laboratory Press, Cold Spring Harbor, NY; Ausubel et al., Short Protocols in Molecular Biology (1999) 4^{th} ed., John Wiley & Sons, Inc.), which are incorporated herein by reference.

As used herein, “and/or” is to be taken as specific disclosure of each of the two or more specified features or components with or without the others. For example, “A, B and/or C” is to be taken as specific disclosure of each (i) A, (ii) B, (iii) C, (iv) A and B, (v) A and C, (vi) B and C and (vii) A and B and C, just as if each is set out individually.

A “polypeptide fragment” herein refers to a portion of a protein, which, for example, may comprise one or more domains of a protein.

A “SARS-CoV-2 Spike polypeptide fragment” or “Spike polypeptide fragment” as described herein refers to a fragment of the SARS-CoV-2 Spike protein. In some embodiments, the fragment includes at least certain domains of the Spike protein, such as the CD1, RBM, and CD2, and optionally also at least a portion of either or both of the N-terminal domain (NT) and C-terminal domain 1 (CTD1), and that does not comprise the complete polypeptide sequence of a native SARS-CoV-2 Spike protein. While in some embodiments, a Spike polypeptide fragment may be described with reference to particular domains of the SARS CoV-2 Spike protein, it is understood that those of ordinary skill in the art can identify the residues comprised within those domains in a new SARS CoV-2 variant Spike protein based on alignments with known SARS CoV-2 Spike protein sequences, such as those of SEQ ID NO: 1 or SEQ ID NO: 2, for example.

When referring to sections of a protein, such as a SARS-CoV-2 Spike protein, the terms “domain” and “region” are used interchangeably herein to refer to particular sections of the protein.

The “N-terminal domain” or “N-terminal region” of the SARS-CoV-2 Spike polypeptide fragment, abbreviated herein “NT” refers to the portion of the SARV-CoV-2 Spike protein comprising residues 316-332 of SEQ ID NO: 1, or the equivalent residues from a SARS-CoV-2 Spike protein that align with residues 316-332 of SEQ ID NO: 1. In some cases, at least a portion of NT is included in a construct herein, such as at least one residue (i.e., position 332 of SEQ ID NO: 1) up to the entire 316-332 NT domain. In describing this and other domains herein, those of ordinary skill in the art will recognize that SEQ ID NO: 1 provides a reference SARS CoV-2 Spike polypeptide amino acid sequence that can be used as a reference sequence to define the bounds of the domains, and that many variant SARS CoV-2 Spike proteins have been identified and continue to be identified. Thus, the bounds of the domains described herein in a newly identified variant Spike protein, for example, can be determined by performing a sequence alignment against a reference SARS CoV-2 Spike polypeptide, such as SEQ ID NO: 1.

The “core domain 1” or “CD1” domain of the SARS-CoV-2 Spike protein refers to the portion comprising residues 333-436 of SEQ ID NO: 1, or the equivalent residues from a SARS-CoV-2 Spike protein that align with residues 333-436 of SEQ ID NO: 1.

The “receptor binding motif” or “RBM” domain of the SARS-CoV-2 Spike protein refers to the part of protein comprising residues 437-508 of SEQ ID NO: 1, or the equivalent residues from a SARS-CoV-2 Spike protein that align with residues 437-508 of SEQ ID NO: 1.

The “CD2” or “core domain 2” of the SARS-CoV-2 Spike protein refers to the portion comprising residues 509-527 of SEQ ID NO: 1, or the equivalent residues from a SARS-CoV-2 Spike protein that align with residues 509-527 of SEQ ID NO: 1.

The “C-terminal domain 1” or “CTD1” region of the SARS-CoV-2 Spike protein refers to the portion comprising residues 528-594 of SEQ ID NO: 1, or the equivalent residues from a SARS-CoV-2 Spike protein that align with residues 528-594 of SEQ ID NO: 1.

In the present disclosure, an “N-terminal tag” or a “C-terminal tag” refers to an amino acid sequence, of at least two amino acids, that is placed at the N-terminus or C-terminus, respectively, of a polypeptide, and that comprises a sequence that is not found at that location in the SARS-CoV-2 Spike protein (in other words, that differs from the native sequence, or is heterologous in sequence). In some instances, such a tag may be used as a means to isolate the SARS-CoV-2 Spike polypeptide fragment during manufacture in cell culture. In some instances, such a tag may be placed at the N- or C-terminus of the Spike polypeptide fragment for other purposes, such as to improve yield during manufacturing, or to add stability to the polypeptide.

A Spike polypeptide fragment may be “fused” to an “N-terminal tag” or “C-terminal tag” herein. In this context, “fused” indicates that the sequence of the N- or C-terminal tag precedes or follows that of the polypeptide fragment either directly or with an intervening linker peptide sequence between the Spike fragment and the tag sequence.

A “fusion protein” or “fusion polypeptide” or “chimeric protein” or “chimeric polypeptide” herein refers to a protein that is made up of amino acid sequences from two different proteins or two different sources, such as, in this case a viral protein and an N- or C-terminal tag sequence (optionally fused directly or via a linker peptide). Accordingly, a Spike polypeptide fragment fused to an N- or C-terminal tag herein constitutes a type of “fusion protein” or “fusion polypeptide” or “chimeric protein” or “chimeric polypeptide,” all of which terms may be used interchangeably when referring to the overall SARS-CoV-2 Spike polypeptide fragment plus N- or C-terminal tag protein construct herein.

In some embodiments herein, the N- and C-terminal residues of the SARS CoV-2 Spike polypeptide fragment are “comprised within an antiparallel beta sheet.” This phrase indicates herein that the N- and the C-terminal residues of the Spike fragment, and/or that the one or two residues immediately adjacent to the N-terminal (i.e., at positions 2 and 3 if the N-terminal residue is at position 1) or the one or two residues immediately adjacent to the C-terminal residue (i.e, at positions 598 and 599 if the C-terminal residue is at position 600, or the like), participate in an antiparallel beta sheet tertiary structure as observed via structural analysis (e.g., by cryo EM, X-ray crystallography, or NMR), or are predicted to participate in an antiparallel beta sheet tertiary structure based on structural analysis of a representative SARS CoV-2 Spike protein and appropriate modeling studies.

The term “host cell” as used herein refers to the particular subject cell transfected with a nucleic acid molecule and the progeny or potential progeny of such a cell. Progeny may not be identical to the parent cell transfected with the nucleic acid molecule due to mutations or environmental influences or developmental steps that may occur in succeeding generations or integration of the nucleic acid molecule into the host cell genome.

As used herein, the “subject” or “patient” or “subject suitable for treatment” or “individual” that may be treated with a fusion polypeptide as described herein is a human, unless specifically noted otherwise (e.g., a mouse subject or the like). In some embodiments, another mammal may be treated, such as a rodent (e.g., a guinea pig, a hamster, a rat, a mouse), murine (e.g., a mouse), canine (e.g., a dog), feline (e.g., a cat), equine (e.g., a horse), a primate, simian (e.g., a monkey or ape), a monkey (e.g., marmoset, baboon, rhesus macaque), an ape (e.g., gorilla, chimpanzee, orangutan, gibbon), or a human. In other embodiments, non-human mammals, especially mammals that are conventionally used as models for demonstrating therapeutic efficacy in humans (e.g., murine, primate, porcine, canine, camels, llamas, or rabbits) may be employed.

As used herein, “and/or” is to be taken as specific disclosure of each of the two or more specified features or components with or without the others. For example, “A, B and/or C” is to be taken as specific disclosure of each (i) A, (ii) B, (iii) C, (iv) A and B, (v) A and C, (vi) B and C and (vii) A and B and C, just as if each is set out individually.

An “immune response” as used herein may include either or both generation of antibodies against a vaccine or antigen administered herein (an “antibody-mediated immune response”), such as anti-Spike protein RBD domain antibodies, in a subject, and a cellular response such as generation, proliferation, or expansion of particular immune cells such as particular types or classes of B cells, long-lived plasma cells (LLPCs), T cells and/or natural killer (NK) cells (an “innate immune response”). An antibody-mediated immune response may be measured, for example, by determining the titer of antibodies against the vaccine or antigen, i.e., anti-RBD antibodies in this case, while an innate immune response may be measured by determining the concentration of particular types of T cells or NK cells or biomarkers related to such cells such as cytokines. For example, in some embodiments, an immune response may be measured by determining a neutralizing titer (NT50) of an antibody directed to the vaccine or antigen, such as of an anti-RBD antibody.

An “adjuvant” as used herein refers to a substance that may be administered to a subject in conjunction with a vaccine or antigen, which may help to improve an immune response against the vaccine or antigen.

The “durability” of a response to a pharmaceutical composition, such as an immune response, as used herein, refers to the length of time that a significant immune response to a vaccine or antigen is observed in a subject. In some cases, durability can be measured by measuring one or more biomarkers of an immune response against the vaccine or antigen at time points following vaccination, such as, for example, titers of antibodies against the vaccine or pathogen antigen, or neutralizing titers of antibodies or serum against live or pseudotyped pathogen, or the presence of certain populations immune cells such as particular T cells, in a sample from a vaccinated subject, such as a blood or serum sample. In some embodiments, durability of an immune response may be measured by determining a neutralizing titer (NT50) of an antibody directed to the vaccine or antigen, such as of an anti-RBD antibody, over time.

The “waning” of a response to a pharmaceutical composition, such as an immune response, as used herein, refers to a significant, measurable reduction in an immune response, such as a reduction in antibody titer against a vaccine or antigen or live or pseudotyped pathogen, or a reduction in particular classes of immune cells; or alternatively, to a response potency that falls below a threshold indicating an effective immune response in a subject, such as an antibody or neutralization titer. Thus, an immune response that “does not wane” over a particular period of time in a subject indicates that biomarkers of immune protection in the subject continue to indicate an effective immune response over that period of time; for example, antibody titers indicate a potent immune response throughout the period, and in some cases also do not decrease significantly over the period. In some embodiments, waning of an immune response may be measured by determining a neutralizing titer (NT50) of an antibody directed to the vaccine or antigen, such as of an anti-RBD antibody, over time.

In many cases, for example, a vaccination or exposure to antigen provokes an initial increase in biomarkers of immune response such as particular antibody levels. These then may decrease over time. In some cases of non-waning immunity, the biomarker levels may decrease but then plateau at a relatively high level, indicating continued immune protection, or may decrease slowly while still remaining high enough to indicate good immune protection over a particular period of time.

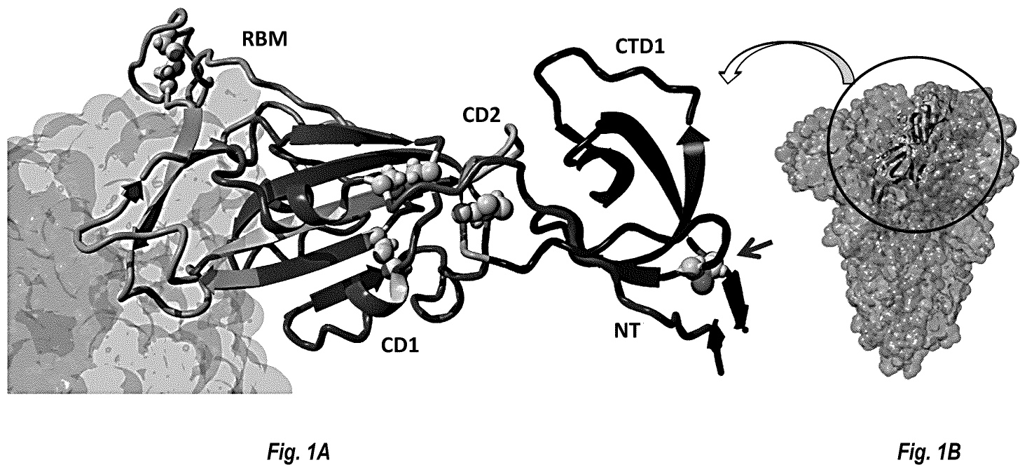

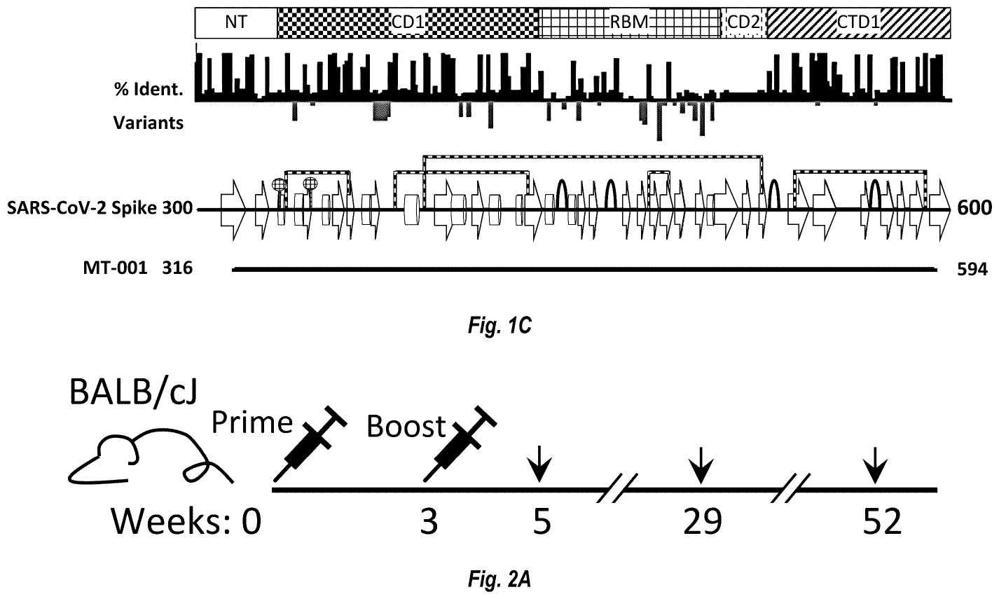

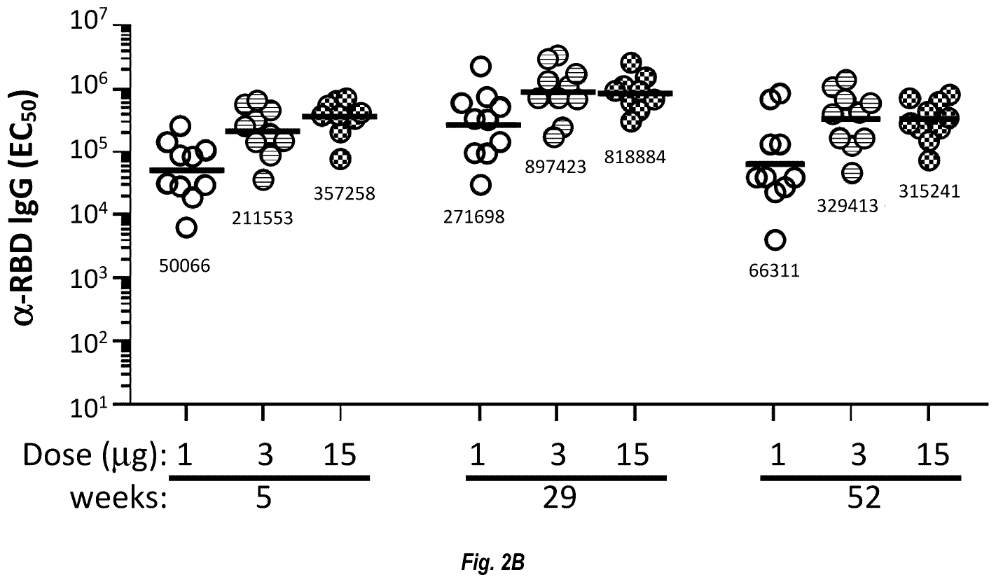

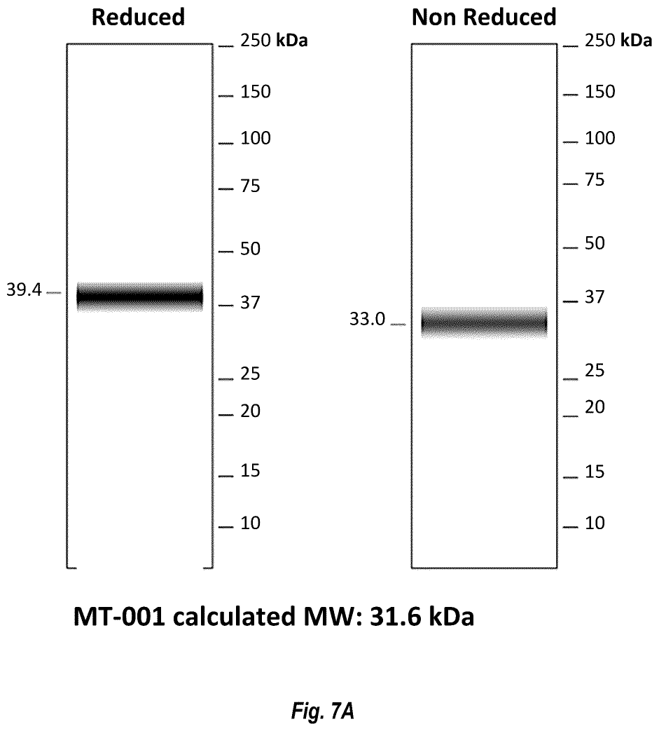

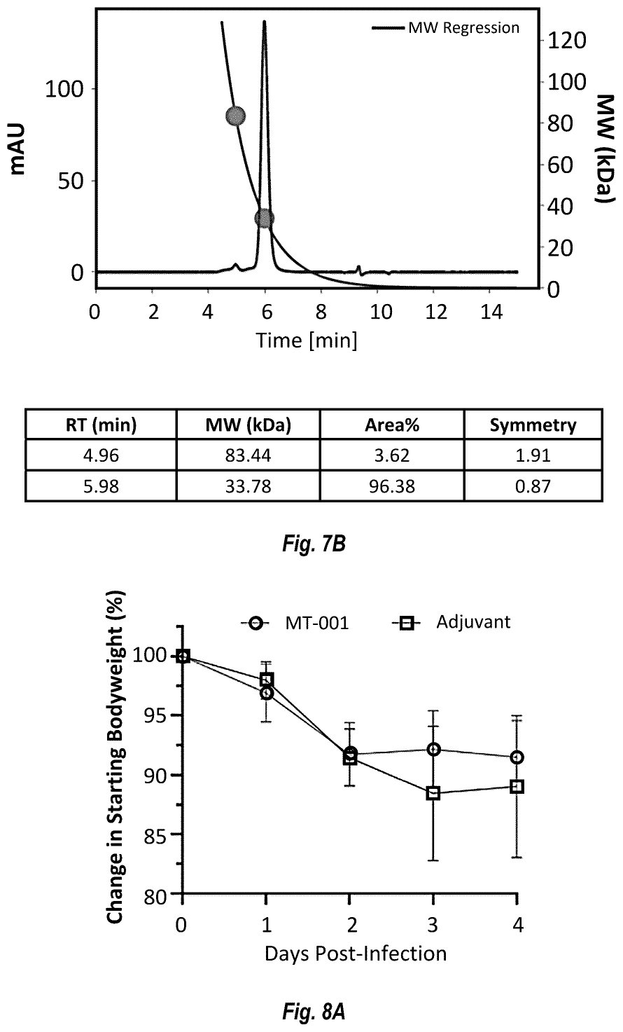

As used herein, “peptidogenicity” refers to the propensity of a protein to efficiently yield a set of diverse peptides which elicit a robust immune response. Various assays exist for measuring peptidogenicity (see, for example, So et al., FIGS. 2c-d; Thai et al., FIG. 7c-f, and Delamarre et al., FIG. 1b-c, 4b-c and 5a-b).

As used herein, a “SARS-CoV-2 peptidogenic protein” or “SARS-CoV-2 peptidogenic fragment” or “SARS-CoV-2 Spike peptidogenic fragment” or similar terms refer to a mutated SARS-CoV-2 encoded protein or fragment thereof that has been modified in its amino acid sequence to alter its conformational dynamics as compared to the SARS-CoV-2 starting protein sequence while maintaining a similar conformation to the SARS-CoV-2 starting protein.

As used herein, “non-surface residues” are residues that are not surface accessible with regard to the 3D structure of a SARS-CoV-2 protein such as a Spike protein, e.g., residues that are buried within the interior of the 3D structure of the native protein. In some embodiments, “non-surface” residues are defined by the method of Lee and Richards (see, e.g., Lee B et al., J. Mol.

Biol. (1971); 55(3):379-IN4. dx.doi.org/10.1016/0022-2836(71)90324-X.), where the relative solvent accessibility of the residue in the native protein is less than 50%, less than 40%, less than 30%, less than 25%, less than 20%, less than 10%, less than 5%, or 0%, or by the same method where the difference between the absolute solvent accessible surface area and the surface area in the fully extended Ala-X-Ala tripeptide (see, e.g., Gready J E et al., Protein Science. (1997); 6(5):983-98. doi: 10.1002/pro.5560060504.) is greater than 40 Å², greater than 50 Å², greater than 60 Å², greater than 70 Å², greater than 80 Å², greater than 90 Å², greater than 100 Å², greater than 110 Å², or greater than 120 Å². In further embodiments, “non-surface” residues are defined as residues with a solvent accessible surface area of less than 10 Å², less than 5 Å², less than 2.5 Å², or less than 1 Å², as calculated by a structural analysis software package familiar to those skilled in the art (e.g., UCSF Chimera (see, e.g., Pettersen E F et al., J. Comput. Chem. (2004); 25(13):1605-12. Epub 2004/07/21.), PyMol (see, e.g., Schrodinger, LLC. The PyMOL Molecular Graphics System, Version 1.8. 2015.), etc.

As used herein, a SARS-CoV-2 peptidogenic protein has a “similar conformation” to a SARS-CoV-2 starting protein if the 3-D structure is sufficiently maintained after mutating non-surface residues of the protein (and, consequently, potentially modifying its overall conformational dynamics) to allow for an antibody to cross react with both the SARS-CoV-2 peptidogenic protein and the SARS-CoV-2 starting protein. “Cross-reactivity” can be measured by a binding assay as described herein or as is well known in the art and is measured as a “binding affinity” which is based on dissociation constants (K_{D}), off rates (k_{off}), and/or on rates (kₒₙ). The SARS-CoV-2 peptidogenic protein does not need to have an identical 3-D structure as the SARS-CoV-2 starting protein; just a sufficiently similar structure displaying similar 3D conformational epitopes (including discontinuous epitopes), that will allow for an antibody to recognize both proteins, even though the binding affinities may be nonidentical.

A “conservative amino acid substitution” is one in which the amino acid residue is replaced with an amino acid residue having a side chain with a similar chemical nature (e.g., size, charge, steric features [e.g., beta-branched vs. non-beta-branched], polarity [hydrophilic vs. hydrophobic], aromatic vs. non-aromatic, etc.). Whether or not a particular substitution is deemed “conservative” may also depend on the structural context in the folded protein in which a substitution occurs.

Amino acid side chains may be chemically similar in one respect but chemically dissimilar in another, and the context may determine which of these properties dominates in terms of how “conservative” (i.e., least disruptive) that particular substitution is. Families of amino acid residues having chemically similar side chains have been defined in the art. These families include amino acids with basic side chains (e.g., lysine, arginine), acidic side chains (e.g., aspartic acid, glutamic acid), uncharged polar side chains (e.g., asparagine, glutamine, serine, threonine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Some side chains have a hybrid character that is pH-dependent in physiologically relevant pH ranges. For example, histidine (pKa ˜6) becomes more positively-charged (basic) below pH 6, and polar but substantially uncharged at pH 7.5 and above. Cysteine (pKa ˜8.5) is substantially uncharged (and not particularly polar) below pH 8, but negatively charged (and acidic) at pH 9. The tyrosine phenolic side chain is also partially ionized and negatively charged at higher pH. Moreover, the local electrostatic environment (context) of the rest of the protein can shift these effective pH values substantially. Moreover, an acidic protein cysteine thiolate side chain can react, via thiol-disulfide exchange involving an intermediary disulfide-containing compound such as oxidized glutathione, with another protein cysteine thiol to form an intramolecular disulfide bond; such bonds are highly hydrophobic (non-polar).

Additionally, both naturally occurring and/or non-naturally occurring amino acids can be used in the SARS-CoV-2 peptidogenic proteins and/or Spike fragment.

Mutations can be introduced in a site-directed fashion or randomly along all or part of the coding sequence. Libraries of mutants can be designed to introduce a single amino acid substitution, two amino acid substitutions, three amino acid substitutions, four amino acid substitutions, and so forth, up to nineteen amino acid substitutions at a given residue site. In still other embodiments, libraries of mutants can be designed to introduce more than nineteen amino acid substitutions (including natural and non-natural amino acids) at a given residue site. In addition, libraries can be combinatorially designed to simultaneously produce multiple mutations at two sites, three sites, four sites, and so on. Following mutagenesis, the encoded protein may routinely be expressed and the conformational dynamics of the encoded protein and/or peptidogenicity can be determined using techniques described herein or by routinely modifying techniques known in the art. The resultant mutant proteins can be screened and evaluated for altered thermodynamic stability or for peptidogenicity or for similar conformation to the SARS-CoV-2 starting protein and/or Spike fragment. Alternatively, the expressed protein “output” from the designed library can be used to immunize an animal without prior screening for protein properties.

In the present disclosure, the term “antibody,” refers to immunoglobulin molecules and immunologically active portions of immunoglobulin molecules, i.e., molecules that contain an antigen binding site. As such, the term antibody encompasses not only whole antibody molecules, but also antibody fragments as well as variants (including derivatives such as fusion proteins) of antibodies and antibody fragments that retain the antigen binding site. In most antibodies, the antigen binding site comprises respective heavy and light chain complementary determining regions (CDRs) and sufficient surrounding framework (FR) regions so that the CDRs are correctly oriented for antigen binding. Examples of molecules which are described by the term “antibody” in this application include, but are not limited to: single chain Fvs (scFvs), Fab fragments, Fab′ fragments, F(ab′)2, disulfide linked Fvs (sdFvs), Fvs, and fragments comprising or alternatively consisting of, either a VL or a VH domain. The term “single chain Fv” or “scFv” as used herein refers to a polypeptide comprising a VL domain of an antibody linked to a VH domain of an antibody. See Carter (2006) Nature Rev. Immunol. 6:243. The antibodies of the present invention may be monospecific, bispecific, trispecific or of greater multispecificity.

Additionally, antibodies of the invention include, but are not limited to, monoclonal, multi-specific, bi-specific, Fc-modified, human, humanized, mouse, or chimeric antibodies, single chain antibodies, camelid antibodies, Fab fragments, F(ab′) fragments, anti-idiotypic (anti-Id) antibodies (including, e.g., anti-Id antibodies to antibodies of the invention), domain antibodies and epitope-binding fragments of any of the above. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule.

### SARS-CoV-2 Spike Polypeptide Fusion Polypeptides

The present disclosure includes, inter alia, certain fusion polypeptides comprising a SARS-CoV-2 Spike polypeptide fragment that comprises at least a portion of the N-terminal domain (NT), domains CD1, RBM, and CD2, and at least a portion of the C-terminal domain 1 (CTD1) as well as a heterologous N- or C-terminal tag comprising at least two, at least three, at least four, at least five, or at least six, or 2, 3, 4, 5, or 6 amino acids that is fused to the N- or C-terminus of the Spike polypeptide fragment.

In some embodiments, the N- and C-terminal residues of the Spike polypeptide fragment, e.g., drawn from the portions of the N-terminal domain and CTD1 included in the fragment, are comprised within an antiparallel beta-sheet. In other words, the N- and C-terminal residues of the Spike fragment are predicted to make contacts with other amino acid residues of the Spike fragment forming an antiparallel beta sheet, either based on molecular modeling with reference to known Spike protein structures, or as observed by cryo-electron microscopy or X-ray crystallography of the Spike polypeptide from which the fragment is taken. In some cases, the antiparallel beta sheet forms between the N- and C-terminal residues of the fragment. Such contacts with other residues at the termini of the fragment may provide stability to the Spike polypeptide fragment in comparison to fragments of different lengths, such as those of shorter length. Without being bound by theory, it is hypothesized that this added stability may contribute to a long-lived durability of immune response of the fusion polypeptides herein in comparison to other RBD Spike polypeptide fragments that are generally shorter in length than those here. (See, e.g., Lazo et al., Vaccine 40: 1162-1169 (2022); Pollet et al., Vaccine 40: 3655-63 (2022); Thuluva et al., eBioMedicine 83: 104217 (2022).) For example, certain constructs comprising an RBD Spike polypeptide fragment have been reported in the literature, but do not comprise residues comprising or aligning with the entire length of SEQ ID NO: 3 herein and/or do not include a portion of both the NT and CTD1 domains, and are not reported to provide the long-lived durability of immune response that certain embodiments herein are shown to provide. Without being bound by theory, one contributing factor to the surprisingly long-lived durability of immune response shown by embodiments herein may be that the N- and C-termini of the Spike polypeptide fragments herein are comprised within an antiparallel beta-sheet, thus providing added stability. It is also possible that the presence of a C-terminal or N-terminal tag may also contribute to the added durability of the immune response. In some cases, for example, a C-terminal EPEA (SEQ ID NO: 103) tag may contribute to the durability of the immune response of the present constructs.

For example, in some embodiments, an antiparallel beta-sheet connects residues at the N- and C-termini of the Spike polypeptide fragment, such as that shown in SEQ ID NO: 7, creating close contacts between residues F318 and G593, N317 and G594, S316 and V595, T315 and S596, Q314 and V597, Y313 and 1598, 1312 and T599, and G311 and P600, among other residues. Thus, in some embodiments, a Spike polypeptide fragment herein includes at least one of these pairs of residues that are comprised within this antiparallel beta sheet. See SEQ ID NO: 7, which encompasses residues 311-600 of SEQ ID NO: 1, as an example. Thus, in some cases, such a Spike polypeptide fragment could begin at a residue from 311 to 316 of SEQ ID NO: 1 such as at 311, 312, 313, 314, 315, or 316 (or at equivalent positions in a SARS-CoV-2 polypeptide that aligns with those residues). In some cases, such a Spike polypeptide fragment could end at a residue from 594 to 600 of SEQ ID NO: 1 such as at 594, 595, 596, 597, 598, 599, or 600 (or at equivalent positions in a SARS-CoV-2 polypeptide that aligns with those residues). In some cases, such a Spike polypeptide fragment could begin at a residue from 311 to 316 of SEQ ID NO: 1 such as at 311, 312, 313, 314, 315, or 316 (or at equivalent positions in a SARS-CoV-2 polypeptide that aligns with those residues) and could end at a residue from 594 to 600 of SEQ ID NO: 1 such as at 594, 595, 596, 597, 598, 599, or 600 (or at equivalent positions in a SARS-CoV-2 polypeptide that aligns with those residues). In some embodiments, the SARS-CoV-2 Spike polypeptide fragment comprises the amino acid sequence of SEQ ID NO: 7, which comprises residues 311-600 of SEQ ID NO: 1.

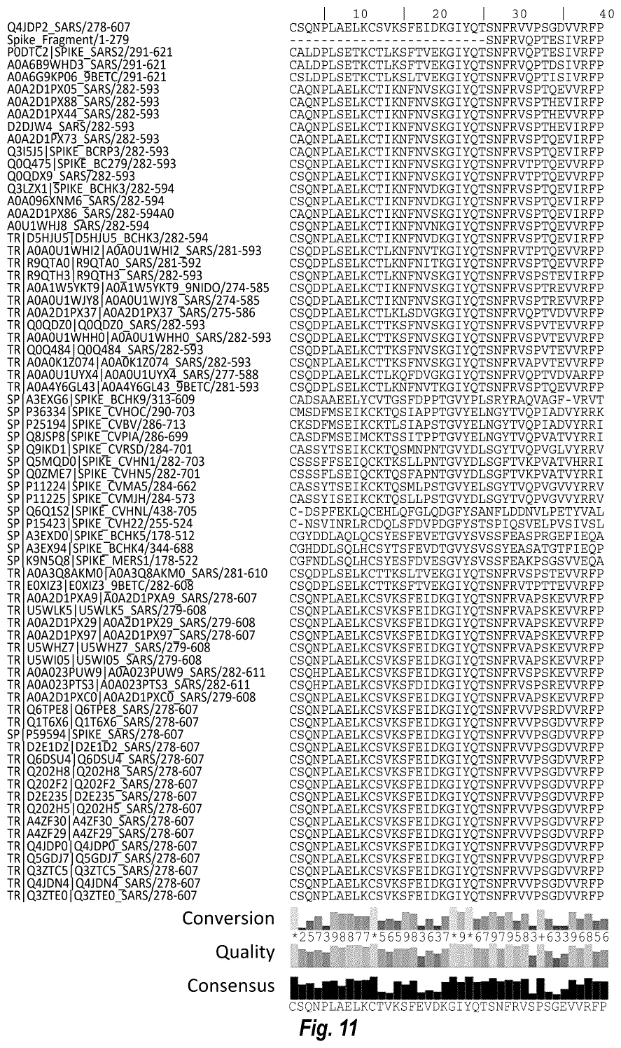

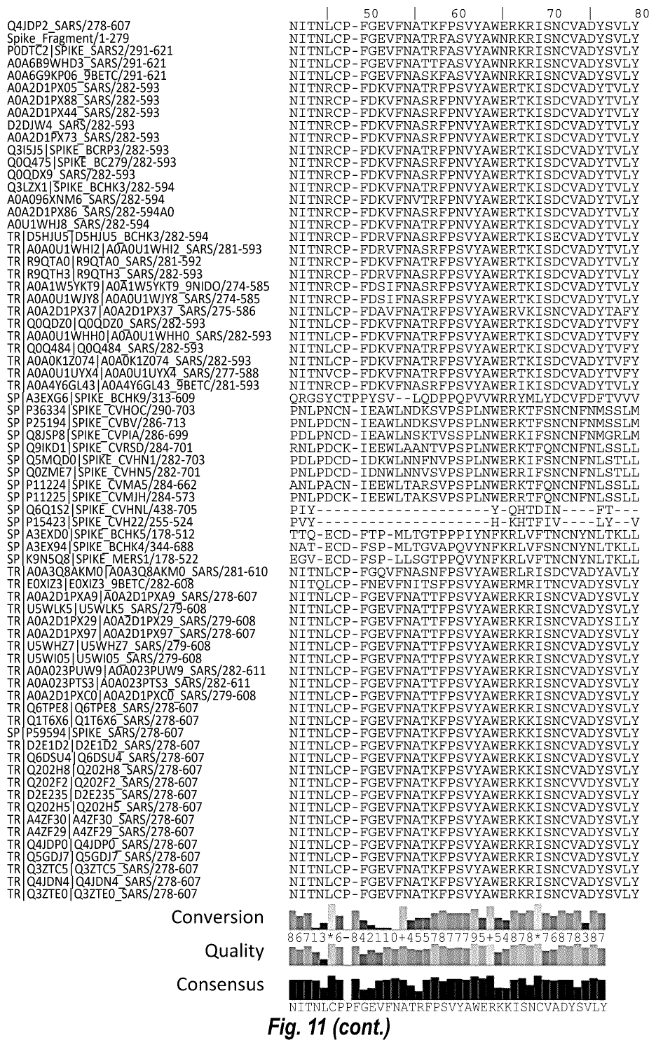

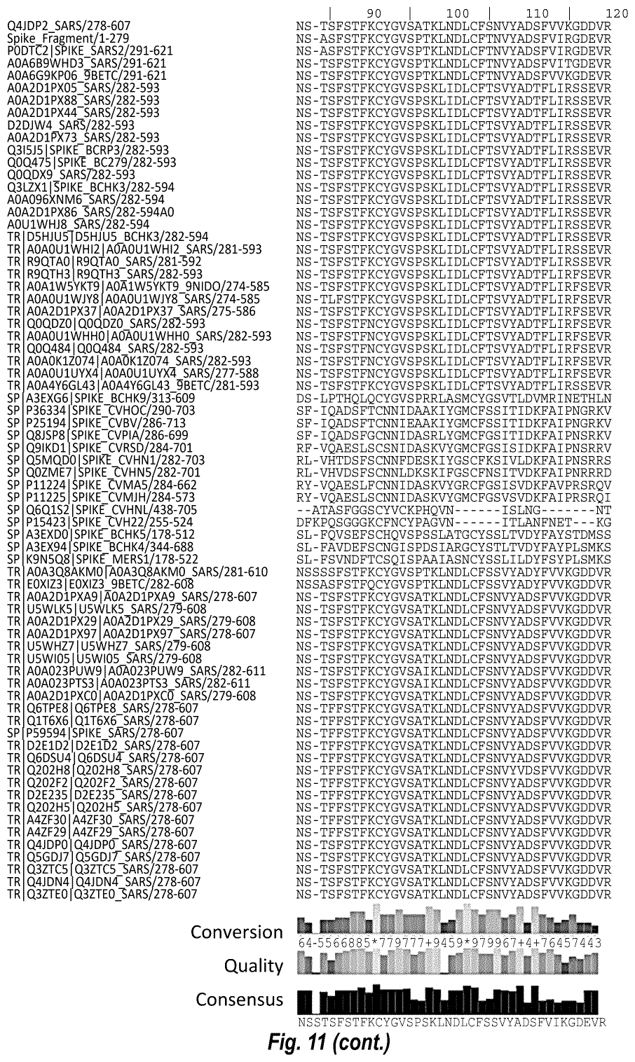

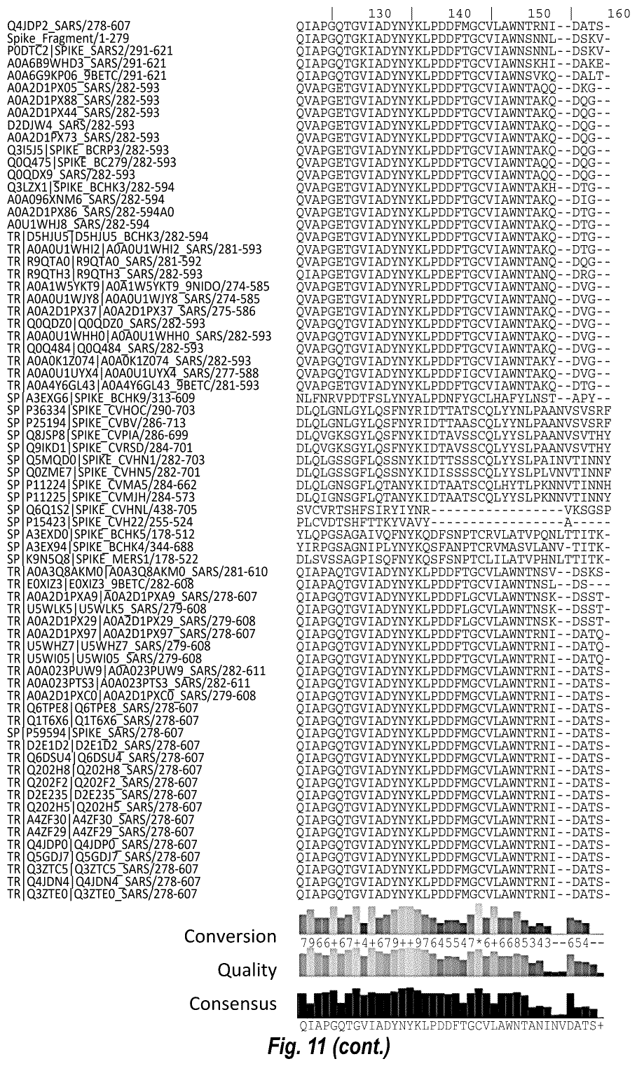

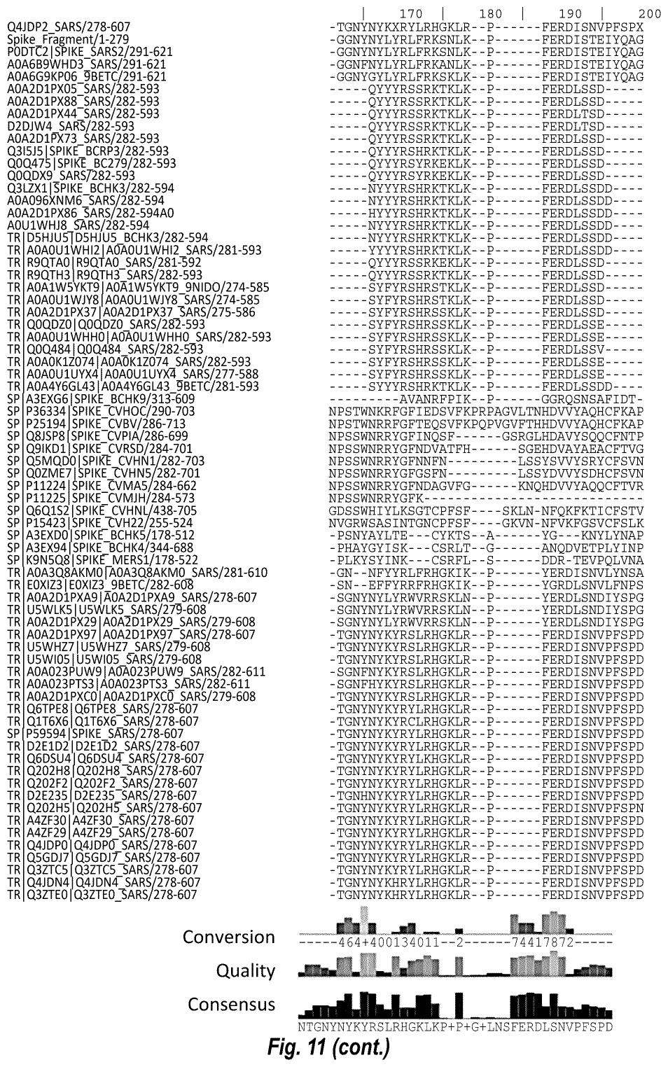

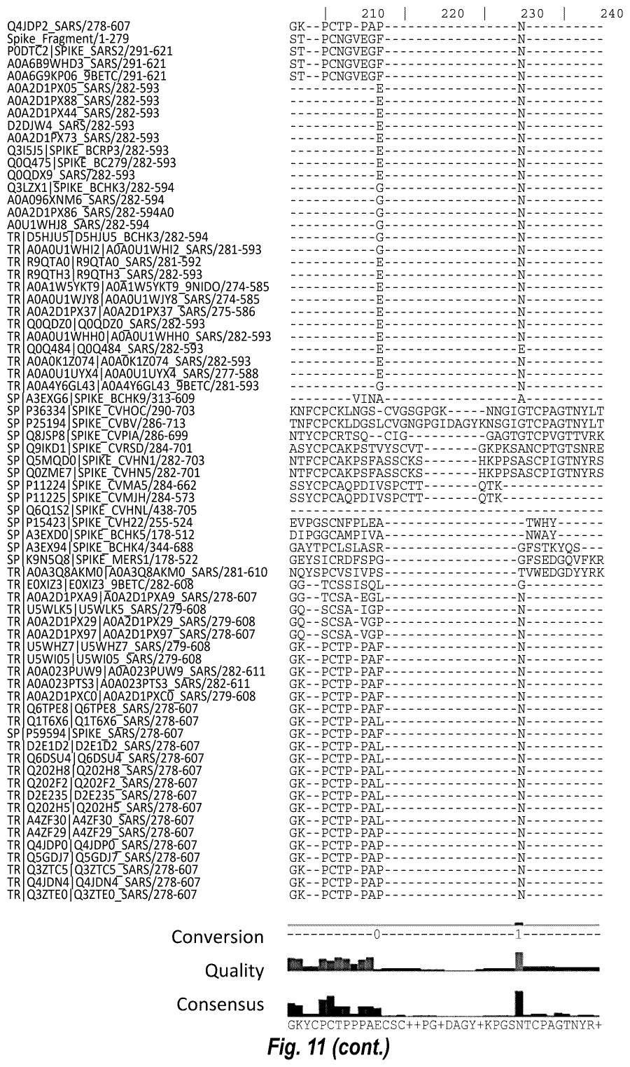

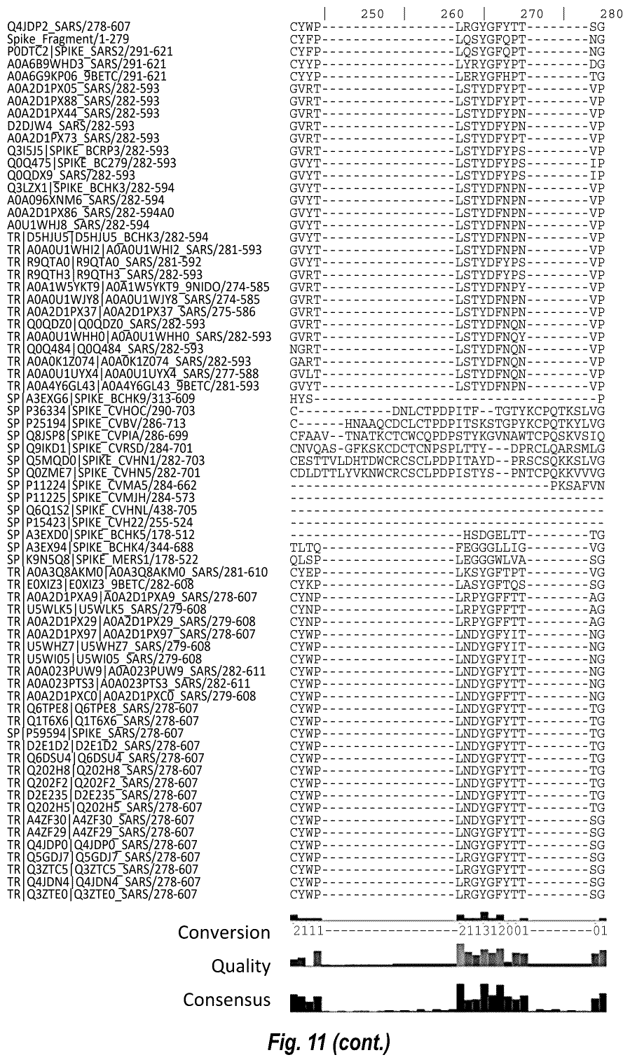

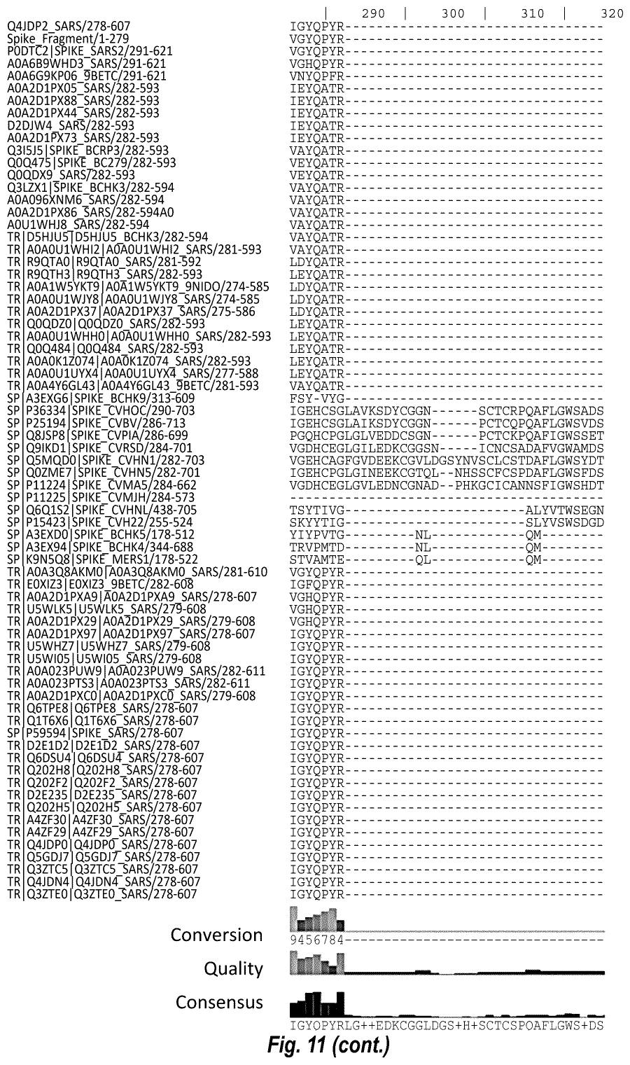

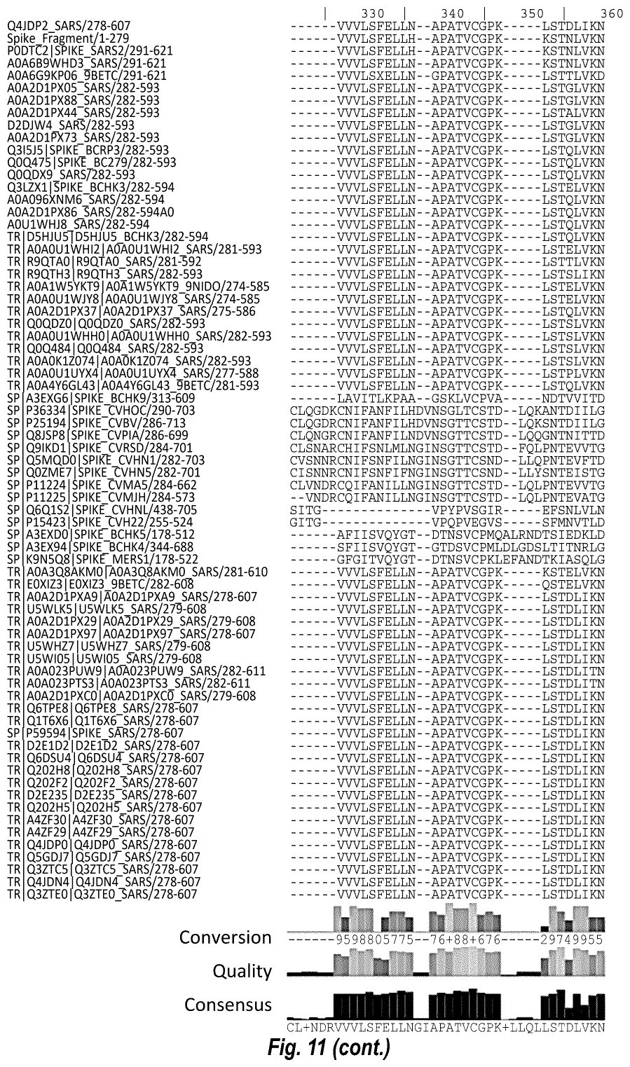

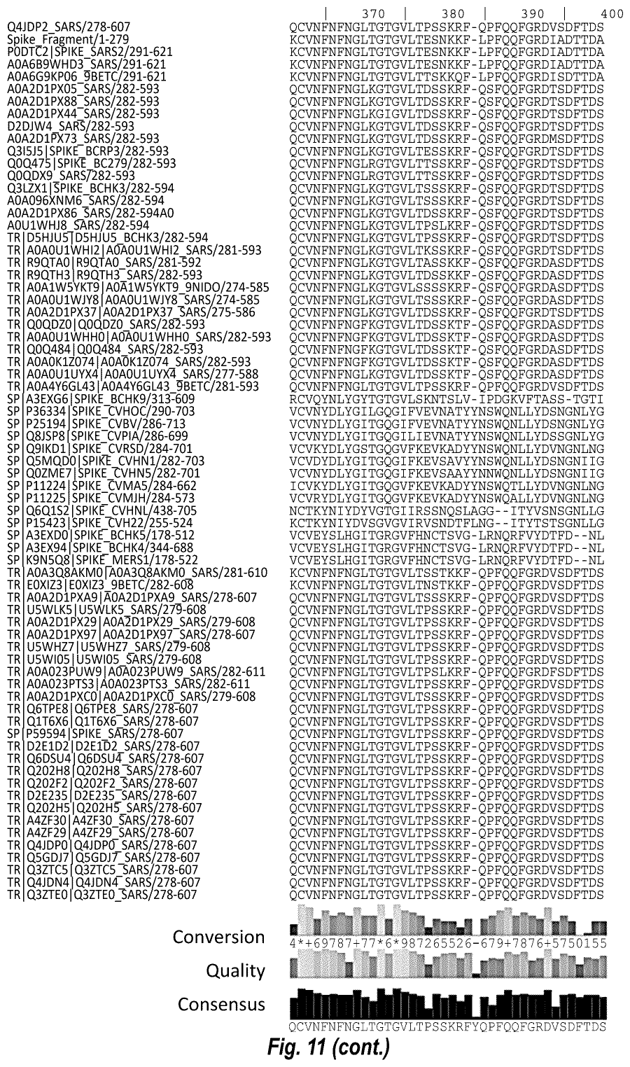

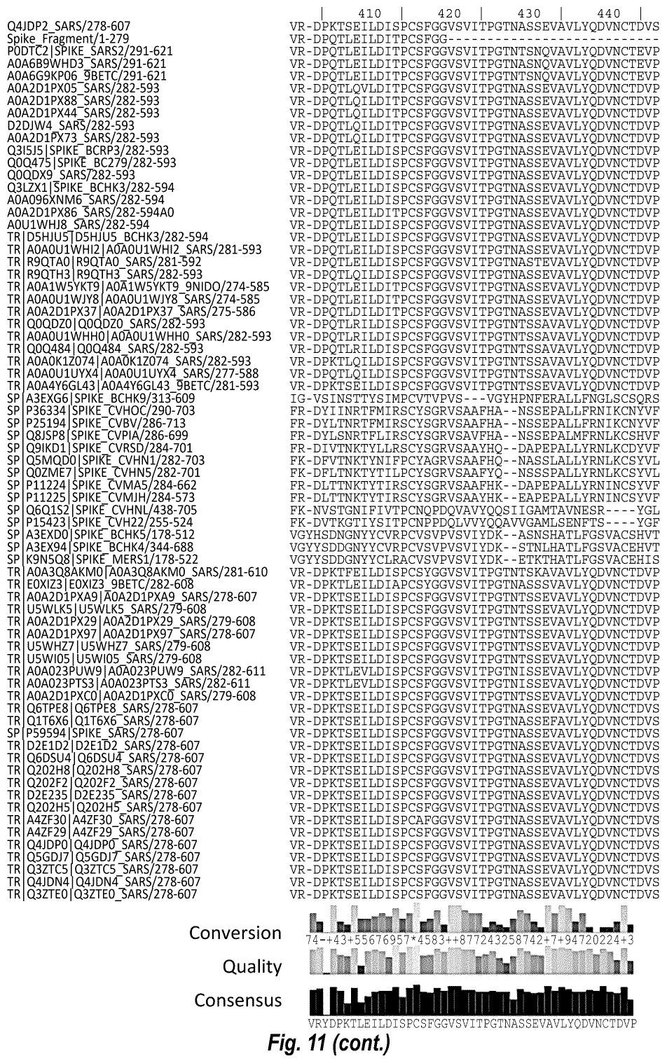

In some embodiments, the SARS-CoV-2 Spike protein fragment comprises residues 316-594 of a SARS-CoV-2 Spike protein as shown in SEQ ID NO: 1, which also corresponds to SEQ ID NO: 3. Positions 316-594 of SEQ ID NO: 1 (SEQ ID NO: 3) include a portion of the N-terminal domain, domains CD1, RBM, and CD2, and a portion of CTD1. In other embodiments, the SARS-CoV-2 Spike protein fragment comprises an equivalent portion of a SARS-CoV-2 Spike protein of a different amino acid sequence that aligns with residues 316-594 of SEQ ID NO: 1. Examples are SEQ ID NO: 5 as well as any of SEQ ID NO: 8 (corresponding to residues 303-580 of SEQ ID NO: 2), and SEQ ID Nos: 9-76 shown in the Table 1 below. Further sequence alignments at positions 300-600 of SARS-CoV-2 Spike polypeptides are shown in FIG. 11 herein. Thus, in some embodiments, a sequence from FIG. 11 that aligns with positions 316-594 of SEQ ID NO: 1 may be selected.

In some embodiments, the SARS-CoV-2 Spike polypeptide fragment comprises or consists of amino acids 316-594 of SEQ ID NO:1 (SEQ ID NO: 3), SEQ ID NO: 5, amino acids 303-580 of SEQ ID NO:2 (SEQ ID NO: 8), or equivalent fragments in other Spike glycoproteins of Coronaviruses, such as those as shown in the below table of sequences (Table 1; SEQ ID NO: 9-76). In some embodiments, the Spike polypeptide fragment comprises or consists of an amino acid sequence that aligns with that of residues 316-594 of SEQ ID NO: 1 and/or residues 303-580 of SEQ ID NO: 2 and/or the amino acid sequence of any one of SEQ ID Nos: 3, 5, 7, or 8 using an appropriate sequence alignment platform.

In some embodiments, an N- or C-tag is placed at the N- and/or C-terminus of the Spike polypeptide fragment. The tag may comprise at least two, at least three, at least four, at least five, at least six, or 2, 3, 4, 5, or 6 amino acid residues. In some embodiments, the tag comprises the amino acid sequence EPEA (SEQ ID NO: 103). In some embodiments, the tag may contribute to protein purification by being recognized by particular antibodies, nanobodies, or other protein capture reagents that specifically bind to the amino acid sequence epitope of the tag, for example. In some embodiments, one or more residues of the tag may further be comprised within the above-described anti-parallel beta sheet involving the N- and C-termini of the Spike polypeptide fragment. In other embodiments, one or more or all of the residues of the tag may be unstructured, i.e., not involved in contacts with residues of the Spike polypeptide fragment.

In some cases, the fusion polypeptide herein comprises a SARS-CoV-2 Spike polypeptide fragment comprising amino acids 316-594 of SEQ ID NO: 1 (SEQ ID NO: 3). In some cases, the fusion polypeptide herein comprises a SARS-CoV-2 Spike polypeptide fragment consisting of amino acids 316-594 of SEQ ID NO: 1 (SEQ ID NO: 3). In some cases, the fusion polypeptide herein comprises a SARS-CoV-2 Spike polypeptide fragment comprising the amino acid sequence of SEQ ID NO: 5. In some cases, the fusion polypeptide herein comprises a SARS-CoV-2 Spike polypeptide fragment consisting of the amino acid sequence of SEQ ID NO: 5. In some cases, the fusion polypeptide herein comprises a SARS-CoV-2 Spike polypeptide fragment comprising an amino acid sequence at least 90%, at least 95%, at least 97%, at least 98%, or at least 99% identical to the amino acid sequence of SEQ ID NO: 3 or SEQ ID NO: 5. In any of those cases, in some embodiments the fusion polypeptide comprises a C-terminal tag, such as a C-terminal tag comprising or consisting of the amino acid sequence of EPEA (SEQ ID NO: 103).

Thus, in some embodiments, the fusion polypeptide comprises or consists of the amino acid sequence of SEQ ID NO: 4. In some embodiments, the fusion polypeptide comprises or consists of the amino acid sequence of SEQ ID NO: 6. In some embodiments, the fusion polypeptide comprises or consists of the amino acid sequence of SEQ ID NO: 78. In some embodiments, the fusion polypeptide comprises or consists of the amino acid sequence of SEQ ID NO: 80. In some embodiments, the fusion polypeptide comprises or consists of the amino acid sequence of SEQ ID NO: 82. In some embodiments, the fusion polypeptide comprises or consists of the amino acid sequence of SEQ ID NO: 84. In some embodiments, the fusion polypeptide comprises or consists of the amino acid sequence of SEQ ID NO: 86. In some embodiments, the fusion polypeptide comprises or consists of the amino acid sequence of SEQ ID NO: 88. In some embodiments, the fusion polypeptide comprises or consists of the amino acid sequence of SEQ ID NO: 90. In some embodiments, the fusion polypeptide comprises or consists of the amino acid sequence of SEQ ID NO: 92. In some embodiments, the fusion polypeptide comprises or consists of the amino acid sequence of SEQ ID NO: 94. In some embodiments, the fusion polypeptide comprises or consists of the amino acid sequence of SEQ ID NO: 96. In some embodiments, the fusion polypeptide comprises or consists of the amino acid sequence of SEQ ID NO: 98. In some embodiments, the fusion polypeptide comprises or consists of the amino acid sequence of SEQ ID NO: 100. In some embodiments, the fusion polypeptide comprises or consists of the amino acid sequence of any one of SEQ ID Nos: 3, 5, or 7-77, 79, 81, 83, 85, 87, 89, 91, 93, 95, 97, or 99 shown below, or any one of the sequences shown in FIG. 11, followed by the amino acid sequence EPEA (SEQ ID NO: 103). In some embodiments, the fusion polypeptide comprises or consists of an amino acid sequence at least 90%, at least 95%, at least 97%, at least 98%, or at least 99% identical to the amino acid sequence of any one of SEQ ID Nos: 3, 5, or 7-77, 79, 81, 83, 85, 87, 89, 91, 93, 95, 97, or 99 shown below, or any one of the sequences shown in FIG. 11, which is followed by the amino acid sequence EPEA (SEQ ID NO: 103). In some embodiments, a composition is provided that comprises a mixture of more than one fusion polypeptide as described above, or a mixture of one or more of the fusion polypeptides described above along with at least one mutant fusion polypeptide as described herein below.

### Exemplary Sequences of SARS-CoV-2 Spike Polypeptide Fragments and Fusion Polypeptides

In some embodiments, a Spike polypeptide fragment herein comprises the amino acid sequence of residues 316-594 of SEQ ID NO: 1 (i.e., SEQ ID NO: 3) below, or alternatively comprises an amino acid sequence of a Spike polypeptide fragment that aligns with residues 316-594 of SEQ ID NO: 1. Examples are depicted in SEQ ID NOs: 5 and 8-76 as shown below, as well as in SEQ ID NOs: 77, 79, 81, 83, 85, 87, 89, 91, 93, 95, 97, and 99 below, and in FIG. 11. In some cases, the SARS-CoV-2 Spike polypeptide fragment is part of a fusion polypeptide, for example, wherein the N- or C-terminus of the Spike polypeptide fragment is fused to a heterologous N- or C-terminal tag comprising at least two, at least three, or at least four amino acids.

As depicted below, residues 316-594 of SEQ ID NO: 1 and the section of SEQ ID NO: 2 equivalent to it based on alignment (residues 303-580 of SEQ ID NO: 2) are highlighted in bold text. These bold sequences are further provided in SEQ ID Nos: 3 and 8. SEQ ID NO: 8 and additional Spike protein fragments that align with residues 316-594 of SEQ ID NO: 1 are provided in the further SEQ ID Nos: 5, 7, 77, 79, 81, 83, 85, 87, 89, 91, 93, 95, 97, and 99 and in SEQ ID NOs: 8-76 of Table 1 below and in FIG. 11. Note that residue numberings 316-594 below in certain sequences such as 77, 79, 81, 83, 85, 87, 89, 91, 93, 95, 97, and 99 are based on alignment of the more recently discovered SARS-CoV-2 spike RBD sequences to the P0DTC2 sequence of SEQ ID NO: 1 below. In some cases, newly discovered RBD sequences comprise deletions or insertions as well as amino acid substitutions, meaning that the actual residue numbering of the complete Spike protein corresponding to the RBD domain may differ from that of P0DTC2. However, to allow for comparison between different RBD domains, the residue numbering of P0DTC2 is retained herein unless explicitly noted otherwise (e.g., in SEQ ID NO: 8). Thus, herein, when particular substitutions are discussed at particular residue locations, those residue locations are based on P0DTC2 residues 316-594 or 311-600 or the like. For example, a substitution at position 365, 402, 511, 519, or 521 means a substitution at the residue corresponding to that residue position in SEQ ID NO: 1.

Certain sequences are as follows:

(Spike protein; Uniprot ID No. P0DTC2 (SPIKE_SARS2)
SEQ ID NO: 1
MFVFLVLLPLVSSQCVNLTTRTQLPPAYTNSFTRGVYYPDKVFRSSVLHSTQDLFLPFFSNVTWF
HAIHVSGTNGTKRFDNPVLPFNDGVYFASTEKSNIIRGWIFGTTLDSKTQSLLIVNNATNVVIKV
CEFQFCNDPFLGVYYHKNNKSWMESEFRVYSSANNCTFEYVSQPFLMDLEGKQGNFKNLREFVFK
NIDGYFKIYSKHTPINLVRDLPQGFSALEPLVDLPIGINITRFQTLLALHRSYLTPGDSSSGWTA
GAAAYYVGYLQPRTFLLKYNENGTITDAVDCALDPLSETKCTLKSFTVEKGIYQTSNFRVQPTES
IVRFPNITNLCPFGEVFNATRFASVYAWNRKRISNCVADYSVLYNSASFSTFKCYGVSPTKLNDL
CFTNVYADSFVIRGDEVRQIAPGQTGKIADYNYKLPDDFTGCVIAWNSNNLDSKVGGNYNYLYRL
FRKSNLKPFERDISTEIYQAGSTPCNGVEGFNCYFPLQSYGFQPTNGVGYQPYRVVVLSFELLHA
PATVCGPKKSTNLVKNKCVNFNFNGLTGTGVLTESNKKFLPFQQFGRDIADTTDAVRDPQTLEIL
DITPCSFGGVSVITPGTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLGAENSVAYSNNSIAIP
TNFTISVTTEILPVSMTKTSVDCTMYICGDSTECSNLLLQYGSFCTQLNRALTGIAVEQDKNTQE
VFAQVKQIYKTPPIKDFGGFNFSQILPDPSKPSKRSFIEDLLFNKVTLADAGFIKQYGDCLGDIA
ARDLICAQKFNGLTVLPPLLTDEMIAQYTSALLAGTITSGWTFGAGAALQIPFAMQMAYRFNGIG
VTQNVLYENQKLIANQFNSAIGKIQDSLSSTASALGKLQDVVNQNAQALNTLVKQLSSNFGAISS
VLNDILSRLDKVEAEVQIDRLITGRLQSLQTYVTQQLIRAAEIRASANLAATKMSECVLGQSKRV
DFCGKGYHLMSFPQSAPHGVVFLHVTYVPAQEKNFTTAPAICHDGKAHFPREGVFVSNGTHWFVT
QRNFYEPQIITTDNTFVSGNCDVVIGIVNNTVYDPLQPELDSFKEELDKYFKNHTSPDVDLGDIS
GINASVVNIQKEIDRLNEVAKNLNESLIDLQELGKYEQYIKWPWYIWLGFIAGLIAIVMVTIMLC
CMTSCCSCLKGCCSCGSCCKFDEDDSEPVLKGVKLHYT
(Spike protein Uniprot ID No. P59594; SPIKE CVHSA)
SEQ ID NO: 2
MFIFLLFLTLTSGSDLDRCTTFDDVQAPNYTQHTSSMRGVYYPDEIFRSDTLYLTQDLFLPFYSN
VTGFHTINHTFGNPVIPFKDGIYFAATEKSNVVRGWVFGSTMNNKSQSVIIINNSTNVVIRACNF
ELCDNPFFAVSKPMGTQTHTMIFDNAFNCTFEYISDAFSLDVSEKSGNFKHLREFVFKNKDGFLY
VYKGYQPIDVVRDLPSGFNTLKPIFKLPLGINITNFRAILTAFSPAQDIWGTSAAAYFVGYLKPT
TFMLKYDENGTITDAVDCSQNPLAELKCSVKSFEIDKGIYQTSNFRVVPSGDVVRFPNITNLCPF
GEVFNATKFPSVYAWERKKISNCVADYSVLYNSTFFSTFKCYGVSATKLNDLCFSNVYADSFVVK
GDDVRQIAPGQTGVIADYNYKLPDDFMGCVLAWNTRNIDATSTGNYNYKYRYLRHGKLRPFERDI
SNVPFSPDGKPCTPPALNCYWPLNDYGFYTTTGIGYQPYRVVVLSFELLNAPATVCGPKLSTDLI
KNQCVNFNFNGLTGTGVLTPSSKRFQPFQQFGRDVSDFTDSVRDPKTSEILDISPCSFGGVSVIT
PGTNASSEVAVLYQDVNCTDVSTAIHADQLTPAWRIYSTGNNVFQTQAGCLIGAEHVDTSYECDI
PIGAGICASYHTVSLLRSTSQKSIVAYTMSLGADSSIAYSNNTIAIPTNFSISITTEVMPVSMAK
TSVDCNMYICGDSTECANLLLQYGSFCTQLNRALSGIAAEQDRNTREVFAQVKQMYKTPTLKYFG
GFNFSQILPDPLKPTKRSFIEDLLFNKVTLADAGFMKQYGECLGDINARDLICAQKFNGLTVLPP
LLTDDMIAAYTAALVSGTATAGWTFGAGAALQIPFAMQMAYRFNGIGVTQNVLYENQKQIANQFN
KAISQIQESLTTTSTALGKLQDVVNQNAQALNTLVKQLSSNFGAISSVLNDILSRLDKVEAEVQI
DRLITGRLQSLQTYVTQQLIRAAEIRASANLAATKMSECVLGQSKRVDFCGKGYHLMSFPQAAPH
GVVFLHVTYVPSQERNFTTAPAICHEGKAYFPREGVFVFNGTSWFITQRNFFSPQIITTDNTFVS
GNCDVVIGIINNTVYDPLQPELDSFKEELDKYFKNHTSPDVDLGDISGINASVVNIQKEIDRLNE
VAKNLNESLIDLQELGKYEQYIKWPWYVWLGFIAGLIAIVMVTILLCCMTSCCSCLKGACSCGSC
CKFDEDDSEPVLKGVKLHYT
(Fragment 316-594 of P0DTC2)
SEQ ID NO: 3
SNFRVQPTESIVRFPNITNLCPFGEVFNATRFASVYAWNRKRISNCVADYSVLYNSASFSTFKCY
GVSPTKLNDLCFTNVYADSFVIRGDEVRQIAPGQTGKIADYNYKLPDDFTGCVIAWNSNNLDSKV
GGNYNYLYRLFRKSNLKPFERDISTEIYQAGSTPCNGVEGFNCYFPLQSYGFQPTNGVGYQPYRV
VVLSFELLHAPATVCGPKKSTNLVKNKCVNFNFNGLTGTGVLTESNKKFLPFQQFGRDIADTTDA
VRDPQTLEILDITPCSFGG
(Fragment 316-594 of P0DTC2 plus EPEA C-terminal tag)
SEQ ID NO: 4
SNFRVQPTESIVRFPNITNLCPFGEVFNATRFASVYAWNRKRISNCVADYSVLYNSASFSTFKCY
GVSPTKLNDLCFTNVYADSFVIRGDEVRQIAPGQTGKIADYNYKLPDDFTGCVIAWNSNNLDSKV
GGNYNYLYRLFRKSNLKPFERDISTEIYQAGSTPCNGVEGFNCYFPLQSYGFQPTNGVGYQPYRV
VVLSFELLHAPATVCGPKKSTNLVKNKCVNFNFNGLTGTGVLTESNKKFLPFQQFGRDIADTTDA
VRDPQTLEILDITPCSFGGEPEA
(Fragment SARS-CoV-2 BA.5 equivalent to fragment 316-594 of SEQ
ID NO: 1)
SEQ ID NO: 5
SNFRVQPTESIVRFPNITNLCPFDEVFNATRFASVYAWNRKRISNCVADYSVLYNFAPFFAFKCY
GVSPTKLNDLCFTNVYADSFVIRGNEVSQIAPGQTGNIADYNYKLPDDFTGCVIAWNSNKLDSKV
GGNYNYRYRLFRKSNLKPFERDISTEIYQAGNKPCNGVAGVNCYFPLQSYGFRPTYGVGHQPYRV
VVLSFELLHAPATVCGPKKSTNLVKNKCVNFNFNGLTGTGVLTESNKKFLPFQQFGRDIADTTDA
VRDPQTLEILDITPCSFGG
(Fragment SARS-CoV-2 BA.5 equivalent to fragment 316-594 of SEQ
ID NO: 1 plus EPEA C-terminal tag)
SEQ ID NO: 6
SNFRVQPTESIVRFPNITNLCPFDEVFNATRFASVYAWNRKRISNCVADYSVLYNFAPFFAFKCY
GVSPTKLNDLCFTNVYADSFVIRGNEVSQIAPGQTGNIADYNYKLPDDFTGCVIAWNSNKLDSKV
GGNYNYRYRLFRKSNLKPFERDISTEIYQAGNKPCNGVAGVNCYFPLQSYGFRPTYGVGHQPYRV
VVLSFELLHAPATVCGPKKSTNLVKNKCVNFNFNGLTGTGVLTESNKKFLPFQQFGRDIADTTDA
VRDPQTLEILDITPCSFGGEPEA
(Residues 311-600 of P0DTC2)
SEQ ID NO: 7
GIYQTSNFRVQPTESIVRFPNITNLCPFGEVFNATRFASVYAWNRKRISNCVADYSVLYNSASFS
TFKCYGVSPTKLNDLCFTNVYADSFVIRGDEVRQIAPGQTGKIADYNYKLPDDFTGCVIAWNSNN
LDSKVGGNYNYLYRLFRKSNLKPFERDISTEIYQAGSTPCNGVEGFNCYFPLQSYGFQPTNGVGY
QPYRVVVLSFELLHAPATVCGPKKSTNLVKNKCVNFNFNGLTGTGVLTESNKKFLPFQQFGRDIA
DTTDAVRDPQTLEILDITPCSFGGVSVITP
(Residues 316-594 of XBB 1.5)
SEQ ID NO: 77
SNFRVQPTESIVRFPNITNLCPFHEVFNATTFASVYAWNRKRISNCVADYSVIYNFAPFFAFKCY
GVSPTKLNDLCFTNVYADSFVIRGNEVSQIAPGQTGNIADYNYKLPDDFTGCVIAWNSNKLDSKP
SGNYNYLYRLFRKSKLKPFERDISTEIYQAGNKPCNGVAGPNCYSPLQSYGFRPTYGVGHQPYRV
VVLSFELLHAPATVCGPKKSTNLVKNKCVNFNFNGLTGTGVLTESNKKFLPFQQFGRDIADTTDA
VRDPQTLEILDITPCSFGG
(Residues 316-594 of XBB 1.5 plus EPEA C-terminal tag)
SEQ ID NO: 78
SNFRVQPTESIVRFPNITNLCPFHEVFNATTFASVYAWNRKRISNCVADYSVIYNFAPFFAFKCY
GVSPTKLNDLCFTNVYADSFVIRGNEVSQIAPGQTGNIADYNYKLPDDFTGCVIAWNSNKLDSKP
SGNYNYLYRLFRKSKLKPFERDISTEIYQAGNKPCNGVAGPNCYSPLQSYGFRPTYGVGHQPYRV
VVLSFELLHAPATVCGPKKSTNLVKNKCVNFNFNGLTGTGVLTESNKKFLPFQQFGRDIADTTDA
VRDPQTLEILDITPCSFGGEPEA
(SARS-CoV-2 BA.2.75 RBD residues 316-594)
SEQ ID NO: 79
SNFRVQPTESIVRFPNITNLCPFHEVFNATRFASVYAWNRKRISNCVADYSVLYNFAPFFAFKCY
GVSPTKLNDLCFTNVYADSFVIRGNEVSQIAPGQTGNIADYNYKLPDDFTGCVIAWNSNKLDSKV
SGNYNYLYRLFRKSKLKPFERDISTEIYQAGNKPCNGVAGFNCYFPLQSYGFRPTYGVGHQPYRV
VVLSFELLHAPATVCGPKKSTNLVKNKCVNFNFNGLTGTGVLTESNKKFLPFQQFGRDIADTTDA
VRDPQTLEILDITPCSFGG
(SARS-CoV-2 BA.2.75 RBD residues 316-594 plus EPEA C-terminal
tag)
SEQ ID NO: 80
SNFRVQPTESIVRFPNITNLCPFHEVFNATRFASVYAWNRKRISNCVADYSVLYNFAPFFAFKCY
GVSPTKLNDLCFTNVYADSFVIRGNEVSQIAPGQTGNIADYNYKLPDDFTGCVIAWNSNKLDSKV
SGNYNYLYRLFRKSKLKPFERDISTEIYQAGNKPCNGVAGFNCYFPLQSYGFRPTYGVGHQPYRV
VVLSFELLHAPATVCGPKKSTNLVKNKCVNFNFNGLTGTGVLTESNKKFLPFQQFGRDIADTTDA
VRDPQTLEILDITPCSFGGEPEA
(SARS-CoV-2 BQ.1 RBD residues 316-594)
SEQ ID NO: 81
SNFRVQPTESIVRFPNITNLCPFDEVFNATRFASVYAWNRKRISNCVADYSVLYNFAPFFAFKCY
GVSPTKLNDLCFTNVYADSFVIRGNEVSQIAPGQTGNIADYNYKLPDDFTGCVIAWNSNKLDSTV
GGNYNYRYRLFRKSKLKPFERDISTEIYQAGNKPCNGVAGVNCYFPLQSYGFRPTYGVGHQPYRV
VVLSFELLHAPATVCGPKKSTNLVKNKCVNFNFNGLTGTGVLTESNKKFLPFQQFGRDIADTTDA
VRDPQTLEILDITPCSFGG
(SARS-CoV-2 BQ.1 RBD residues 316-594 plus EPEA C-terminal tag)
SEQ ID NO: 82
SNFRVQPTESIVRFPNITNLCPFDEVFNATRFASVYAWNRKRISNCVADYSVLYNFAPFFAFKCY
GVSPTKLNDLCFTNVYADSFVIRGNEVSQIAPGQTGNIADYNYKLPDDFTGCVIAWNSNKLDSTV
GGNYNYRYRLFRKSKLKPFERDISTEIYQAGNKPCNGVAGVNCYFPLQSYGFRPTYGVGHQPYRV
VVLSFELLHAPATVCGPKKSTNLVKNKCVNFNFNGLTGTGVLTESNKKFLPFQQFGRDIADTTDA
VRDPQTLEILDITPCSFGGEPEA
(SARS-CoV-2 XBB.1.16 RBD residues 316-594)
SEQ ID NO: 83
SNFRVQPTESIVRFPNITNLCPFHEVFNATTFASVYAWNRKRISNCVADYSVIYNFAPFFAFKCY
GVSPTKLNDLCFTNVYADSFVIRGNEVSQIAPGQTGNIADYNYKLPDDFTGCVIAWNSNKLDSKP
SGNYNYLYRLFRKSKLKPFERDISTEIYQAGNRPCNGVAGPNCYSPLQSYGFRPTYGVGHQPYRV
VVLSFELLHAPATVCGPKKSTNLVKNKCVNFNFNGLTGTGVLTESNKKFLPFQQFGRDIADTTDA
VRDPQTLEILDITPCSFGG
(SARS-CoV-2 XBB.1.16 RBD residues 316-594 plus C-terminal EPEA
tag)
SEQ ID NO: 84
SNFRVQPTESIVRFPNITNLCPFHEVFNATTFASVYAWNRKRISNCVADYSVIYNFAPFFAFKCY
GVSPTKLNDLCFTNVYADSFVIRGNEVSQIAPGQTGNIADYNYKLPDDFTGCVIAWNSNKLDSKP
SGNYNYLYRLFRKSKLKPFERDISTEIYQAGNRPCNGVAGPNCYSPLQSYGFRPTYGVGHQPYRV
VVLSFELLHAPATVCGPKKSTNLVKNKCVNFNFNGLTGTGVLTESNKKFLPFQQFGRDIADTTDA
VRDPQTLEILDITPCSFGGEPEA
(SARS-CoV-2 CH.1.1 RBD residues 316-594)
SEQ ID NO: 85
SNFRVQPTESIVRFPNITNLCPFHEVFNATTFASVYAWNRKRISNCVADYSVLYNFAPFFAFKCY
GVSPTKLNDLCFTNVYADSFVIRGNEVSQIAPGQTGNIADYNYKLPDDFTGCVIAWNSNKLDSTV
SGNYNYRYRLFRKSKLKPFERDISTEIYQAGNKPCNGVAGSNCYFPLQSYGFRPTYGVGHQPYRV
VVLSFELLHAPATVCGPKKSTNLVKNKCVNFNFNGLTGTGVLTESNKKFLPFQQFGRDIADTTDA
VRDPQTLEILDITPCSFGG
(SARS-CoV-2 CH.1.1 RBD residues 316-594 plus EPEA C-terminal tag)
SEQ ID NO: 86
SNFRVQPTESIVRFPNITNLCPFHEVFNATTFASVYAWNRKRISNCVADYSVLYNFAPFFAFKCY
GVSPTKLNDLCFTNVYADSFVIRGNEVSQIAPGQTGNIADYNYKLPDDFTGCVIAWNSNKLDSTV
SGNYNYRYRLFRKSKLKPFERDISTEIYQAGNKPCNGVAGSNCYFPLQSYGFRPTYGVGHQPYRV
VVLSFELLHAPATVCGPKKSTNLVKNKCVNFNFNGLTGTGVLTESNKKFLPFQQFGRDIADTTDA
VRDPQTLEILDITPCSFGGEPEA
(SARS-CoV-2 XBB.1.9 RBD residues 316-594)
SEQ ID NO: 87
SNFRVQPTESIVRFPNITNLCPFHEVFNATTFASVYAWNRKRISNCVADYSVIYNFAPFFAFKCY
GVSPTKLNDLCFTNVYADSFVIRGNEVSQIAPGQTGNIADYNYKLPDDFTGCVIAWNSNKLDSKP
SGNYNYLYRLFRKSKLKPFERDISTEIYQAGNKPCNGVAGSNCYSPLQSYGFRPTYGVGHQPYRV
VVLSFELLHAPATVCGPKKSTNLVKNKCVNFNFNGLTGTGVLTESNKKFLPFQQFGRDIADTTDA
VRDPQTLEILDITPCSFGG
(SARS-CoV-2 XBB.1.9 RBD residues 316-594 plus EPEA C-terminal
tag)
SEQ ID NO: 88
SNFRVQPTESIVRFPNITNLCPFHEVFNATTFASVYAWNRKRISNCVADYSVIYNFAPFFAFKCY
GVSPTKLNDLCFTNVYADSFVIRGNEVSQIAPGQTGNIADYNYKLPDDFTGCVIAWNSNKLDSKP
SGNYNYLYRLFRKSKLKPFERDISTEIYQAGNKPCNGVAGSNCYSPLQSYGFRPTYGVGHQPYRV
VVLSFELLHAPATVCGPKKSTNLVKNKCVNFNFNGLTGTGVLTESNKKFLPFQQFGRDIADTTDA
VRDPQTLEILDITPCSFGGEPEA
(SARS-CoV-2 XBB.2.3 RBD residues 316-594)
SEQ ID NO: 89
SNFRVQPTESIVRFPNITNLCPFHEVFNATTFASVYAWNRKRISNCVADYSVIYNFAPFFAFKCY
GVSPTKLNDLCFTNVYADSFVIRGNEVSQIAPGQTGNIADYNYKLPDDFTGCVIAWNSNKLDSKP
SGNYNYLYRLFRKSKLKPFERDISTEIYQAGNKPCNGVAGPNCYSPLQSYGFRPTYGVGHQPYRV
VVLSFELLHASATVCGPKKSTNLVKNKCVNFNFNGLTGTGVLTESNKKFLPFQQFGRDIADTTDA
VRDPQTLEILDITPCSFGG
(SARS-CoV-2 XBB.2.3 RBD residues 316-594 plus EPEA C-terminal
tag)
SEQ ID NO: 90
SNFRVQPTESIVRFPNITNLCPFHEVFNATTFASVYAWNRKRISNCVADYSVIYNFAPFFAFKCY
GVSPTKLNDLCFTNVYADSFVIRGNEVSQIAPGQTGNIADYNYKLPDDFTGCVIAWNSNKLDSKP
SGNYNYLYRLFRKSKLKPFERDISTEIYQAGNKPCNGVAGPNCYSPLQSYGFRPTYGVGHQPYRV
VVLSFELLHASATVCGPKKSTNLVKNKCVNFNFNGLTGTGVLTESNKKFLPFQQFGRDIADTTDA
VRDPQTLEILDITPCSFGGEPEA
(SARS-CoV-2 EG.5.1 RBD residues 316-594)
SEQ ID NO: 91
SNFRVQPTESIVRFPNITNLCPFHEVFNATTFASVYAWNRKRISNCVADYSVIYNFAPFFAFKCY
GVSPTKLNDLCFTNVYADSFVIRGNEVSQIAPGQTGNIADYNYKLPDDFTGCVIAWNSNKLDSKP
SGNYNYLYRLLRKSKLKPFERDISTEIYQAGNKPCNGVAGPNCYSPLQSYGFRPTYGVGHQPYRV
VVLSFELLHAPATVCGPKKSTNLVKNKCVNFNFNGLTGTGVLTESNKKFLPFQQFGRDIADTTDA
VRDPQTLEILDITPCSFGG
(SARS-CoV-2 EG.5.1 RBD residues 316-594 plus C-terminal EPEA tag)
SEQ ID NO: 92
SNFRVQPTESIVRFPNITNLCPFHEVFNATTFASVYAWNRKRISNCVADYSVIYNFAPFFAFKCY
GVSPTKLNDLCFTNVYADSFVIRGNEVSQIAPGQTGNIADYNYKLPDDFTGCVIAWNSNKLDSKP
SGNYNYLYRLLRKSKLKPFERDISTEIYQAGNKPCNGVAGPNCYSPLQSYGFRPTYGVGHQPYRV
VVLSFELLHAPATVCGPKKSTNLVKNKCVNFNFNGLTGTGVLTESNKKFLPFQQFGRDIADTTDA
VRDPQTLEILDITPCSFGGEPEA
(SARS-CoV-2 XBB.1.5.70 RBD residues 316-594)
SEQ ID NO: 93
SNFRVQPTESIVRFPNITNLCPFHEVFNATTFASVYAWNRKRISNCVADYSVIYNFAPFFAFKCY
GVSPTKLNDLCFTNVYADSFVIRGNEVSQIAPGQTGNIADYNYKLPDDFTGCVIAWNSNKLDSKP
SGNYNYLYRFLRKSKLKPFERDISTEIYQAGNKPCNGVAGPNCYSPLQSYGFRPTYGVGHQPYRV
VVLSFELLHAPATVCGPKKSTNLVKNKCVNFNFNGLTGTGVLTESNKKFLPFQQFGRDIADTTDA
VRDPQTLEILDITPCSFGG
(SARS-CoV-2 XBB.1.5.70 RBD residues 316-594 plus EPEA C-terminal
tag)
SEQ ID NO: 94
SNFRVQPTESIVRFPNITNLCPFHEVFNATTFASVYAWNRKRISNCVADYSVIYNFAPFFAFKCY
GVSPTKLNDLCFTNVYADSFVIRGNEVSQIAPGQTGNIADYNYKLPDDFTGCVIAWNSNKLDSKP
SGNYNYLYRFLRKSKLKPFERDISTEIYQAGNKPCNGVAGPNCYSPLQSYGFRPTYGVGHQPYRV
VVLSFELLHAPATVCGPKKSTNLVKNKCVNFNFNGLTGTGVLTESNKKFLPFQQFGRDIADTTDA
VRDPQTLEILDITPCSFGGEPEA
(SARS-CoV-2 HK.3 RBD residues 316-594)
SEQ ID NO: 95
SNFRVQPTESIVRFPNITNLCPFHEVFNATTFASVYAWNRKRISNCVADYSVIYNFAPFFAFKCY
GVSPTKLNDLCFTNVYADSFVIRGNEVSQIAPGQTGNIADYNYKLPDDFTGCVIAWNSNKLDSKP
SGNYNYLYRFLRKSKLKPFERDISTEIYQAGNKPCNGVAGPNCYSPLQSYGFRPTYGVGHQPYRV
VVLSFELLHAPATVCGPKKSTNLVKNKCVNFNFNGLTGTGVLTESNKKFLPFQQFGRDIADTTDA
VRDPQTLEILDITPCSFGG
(SARS-CoV-2 HK.3 RBD residues 316-594 plus EPEA C-terminal tag)
SEQ ID NO: 96
SNFRVQPTESIVRFPNITNLCPFHEVFNATTFASVYAWNRKRISNCVADYSVIYNFAPFFAFKCY
GVSPTKLNDLCFTNVYADSFVIRGNEVSQIAPGQTGNIADYNYKLPDDFTGCVIAWNSNKLDSKP
SGNYNYLYRFLRKSKLKPFERDISTEIYQAGNKPCNGVAGPNCYSPLQSYGFRPTYGVGHQPYRV
VVLSFELLHAPATVCGPKKSTNLVKNKCVNFNFNGLTGTGVLTESNKKFLPFQQFGRDIADTTDA
VRDPQTLEILDITPCSFGGEPEA
(SARS-CoV-2 BA.2.86 RBD residues 316-594)
SEQ ID NO: 97
SNFRVQPTESIVRFPNVTNLCPFHEVFNATRFASVYAWNRTRISNCVADYSVLYNFAPFFAFKCY
GVSPTKLNDLCFTNVYADSFVIKGNEVSQIAPGQTGNIADYNYKLPDDFTGCVIAWNSNKLDSKH
SGNYDYWYRLFRKSKLKPFERDISTEIYQAGNKPCKGKGPNCYFPLQSYGFRPTYGVGHQPYRVV
VLSFELLHAPATVCGPKKSTNLVKNKCVNFNFNGLTGTGVLTKSNKKFLPFQQFGRDIVDTTDAV
RDPQTLEILDITPCSFGG
(SARS-CoV-2 BA.2.86 RBD residues 316-594 plus EPEA C-terminal
tag)
SEQ ID NO: 98
SNFRVQPTESIVRFPNVTNLCPFHEVFNATRFASVYAWNRTRISNCVADYSVLYNFAPFFAFKCY
GVSPTKLNDLCFTNVYADSFVIKGNEVSQIAPGQTGNIADYNYKLPDDFTGCVIAWNSNKLDSKH
SGNYDYWYRLFRKSKLKPFERDISTEIYQAGNKPCKGKGPNCYFPLQSYGFRPTYGVGHQPYRVV
VLSFELLHAPATVCGPKKSTNLVKNKCVNFNFNGLTGTGVLTKSNKKFLPFQQFGRDIVDTTDAV
RDPQTLEILDITPCSFGGEPEA
(SARS-CoV-2 JN.1 RBD residues 316-594)
SEQ ID NO: 99
SNFRVQPTESIVRFPNVTNLCPFHEVFNATRFASVYAWNRTRISNCVADYSVLYNFAPFFAFKCY
GVSPTKLNDLCFTNVYADSFVIKGNEVSQIAPGQTGNIADYNYKLPDDFTGCVIAWNSNKLDSKH
SGNYDYWYRSFRKSKLKPFERDISTEIYQAGNKPCKGKGPNCYFPLQSYGFRPTYGVGHQPYRVV
VLSFELLHAPATVCGPKKSTNLVKNKCVNFNFNGLTGTGVLTKSNKKFLPFQQFGRDIVDTTDAV
RDPQTLEILDITPCSFGG
(SARS-CoV-2 JN.1 RBD residues 316-594 plus EPEA C-terminal tag)
SEQ ID NO: 100
SNFRVQPTESIVRFPNVTNLCPFHEVFNATRFASVYAWNRTRISNCVADYSVLYNFAPFFAFKCY
GVSPTKLNDLCFTNVYADSFVIKGNEVSQIAPGQTGNIADYNYKLPDDFTGCVIAWNSNKLDSKH
SGNYDYWYRSFRKSKLKPFERDISTEIYQAGNKPCKGKGPNCYFPLQSYGFRPTYGVGHQPYRVV
VLSFELLHAPATVCGPKKSTNLVKNKCVNFNFNGLTGTGVLTKSNKKFLPFQQFGRDIVDTTDAV
RDPQTLEILDITPCSFGGEPEA

TABLE 1
Additional SARS-CoV-2 Spike RBD Sequences
with their original residue numbering in column 2

| Additional | | | |
|---|---|---|---|
| Spike | Residues | | |
| Protein | aligining | | |
| Fragment | to P0DTC2 | SEQ | |
| Accession | residues | ID | |
| No. | 316-594 | NO: | Spike Fragment Sequence |
| P59594 | 303-580 | 8 | SNFRVVPSGDVVRFPNITNLCPFGEVFNATKFPSVYAWERKKISNCVADYSVLYNSTFFSTFKCYGVSAT |
| | | | KLNDLCFSNVYADSFVVKGDDVRQIAPGQTGVIADYNYKLPDDFMGCVLAWNTRNIDATSTGNYNYKYRY |
| | | | LRHGKLRPFERDISNVPFSPDGKPCTPPALNCYWPLNDYGFYTTTGIGYQPYRVVVLSFELLNAPATVCG |
| | | | PKLSTDLIKNQCVNFNFNGLTGTGVLTPSSKRFQPFQQFGRDVSDFTDSVRDPKTSEILDISPCSFGG |
| A0A6B9WHD3 | 316-594 | 9 | SNFRVQPTDSIVRFPNITNLCPFGEVFNATTFASVYAWNRKRISNCVADYSVLYNSTSFSTFKCYGVSPT |
| | | | KLNDLCFTNVYADSFVITGDEVRQIAPGQTGKIADYNYKLPDDFTGCVIAWNSKHIDAKEGGNFNYLYRL |
| | | | FRKANLKPFERDISTEIYQAGSKPCNGQTGLNCYYPLYRYGFYPTDGVGHQPYRVVVLSFELLNAPATVC |
| | | | GPKKSTNLVKNKCVNFNFNGLTGTGVLTESNKKFLPFQQFGRDIADTTDAVRDPQTLEILDITPCSFGG |
| A0A6G9KP06 | 316-594 | 10 | SNFRVQPTISIVRFPNITNLCPFGEVFNASKFASVYAWNRKRISNCVADYSVLYNSTSFSTFKCYGVSPT |
| | | | KLNDLCFTNVYADSFVVKGDEVRQIAPGQTGVIADYNYKLPDDFTGCVIAWNSVKQDALTGGNYGYLYRL |
| | | | FRKSKLKPFERDISTEIYQAGSTPCNGQVGLNCYYPLERYGFHPTTGVNYQPFRVVVLSXELLNGPATVC |
| | | | GPKLSTTLVKDKCVNFNFNGLTGTGVLTTSKKQFLPFQQFGRDISDTTDAVRDPQTLEILDITPCSFGG |
| A0A2D1PX05 | 307-566 | 11 | SNFRVSPTQEVVRFPNITNRCPFDKVFNATRFPSVYAWERTKISDCVADYTVLYNSTSFSTFKCYGVSPS |
| | | | KLIDLCFTSVYADTFLIRSSEVRQVAPGETGVIADYNYKLPDDFTGCVIAWNTAQQDKGQYYYRSSRKTK |
| | | | LKPFERDLSSDENGVRTLSTYDFYPTVPIEYQATRVVVLSFELLNAPATVCGPKLSTGLVKNQCVNFNFN |
| | | | GLKGTGVLTDSSKRFQSFQQFGRDTSDFTDSVRDPQTLQVLDITPCSFGG |
| A0A2D1PX88 | 307-566 | 12 | SNFRVSPTHEVIRFPNITNRCPFDKVFNASRFPNVYAWERTKISDCVADYTVLYNSTSFSTFKCYGVSPS |
| | | | KLIDLCFTSVYADTFLIRSSEVRQVAPGETGVIADYNYKLPDDFTGCVIAWNTAKQDQGQYYYRSSRKTK |
| | | | LKPFERDLSSDENGVRTLSTYDFYPTVPIEYQATRVVVLSFELLNAPATVCGPKLSTGLVKNQCVNFNFN |
| | | | GLKGTGVLTDSSKRFQSFQQFGRDTSDFTDSVRDPQTLQILDITPCSFGG |
| A0A2D1PX44 | 307-566 | 13 | SNFRVSPTHEVVRFPNITNRCPFDKVFNASRFPNVYAWERTKISDCVADYTVLYNSTSFSTFKCYGVSPS |
| | | | KLIDLCFTSVYADTFLIRSSEVRQVAPGETGVIADYNYKLPDDFTGCVIAWNTAKQDQGQYYYRSSRKTK |
| | | | LKPFERDLTSDENGVRTLSTYDFYPNVPIEYQATRVVVLSFELLNAPATVCGPKLSTALVKNQCVNFNFN |
| | | | GLKGIGVLTDSSKRFQSFQQFGRDTSDFTDSVRDPQTLQILDITPCSFGG |
| D2DJW4 | 307-566 | 14 | SNFRVSPTHEVIRFPNITNRCPFDKVFNASRFPNVYAWERTKISDCVADYTVLYNSTSFSTFKCYGVSPS |
| | | | KLIDLCFTSVYADTFLIRSSEVRQVAPGETGVIADYNYKLPDDFTGCVIAWNTAKQDQGQYYYRSSRKTK |
| | | | LKPFERDLTSDENGVRTLSTYDFYPNVPIEYQATRVVVLSFELLNAPATVCGPKLSTGLVKNQCVNFNFN |
| | | | GLKGTGVLTDSSKRFQSFQQFGRDTSDFTDSVRDPQTLQILDITPCSFGG |
| A0A2D1PX73 | 307-566 | 15 | SNFRVSPTQEVIRFPNITNRCPFDKVFNASRFPNVYAWERTKISDCVADYTVLYNSTSFSTFKCYGVSPS |
| | | | KLIDLCFTSVYADTFLIRSSEVRQVAPGETGVIADYNYKLPDDFTGCVIAWNTAKQDQGQYYYRSSRKTK |
| | | | LKPFERDLSSDENGVRTLSTYDFYPTVPIEYQATRVVVLSFELLNAPATVCGPKLSTGLVKNQCVNFNFN |
| | | | GLKGTGVLTDSSKRFQSFQQFGRDMSDFTDSVRDPQTLQILDITPCSFGG |
| Q3I5J5 | 307-566 | 16 | SNFRVSPTQEVIRFPNITNRCPFDKVFNATRFPNVYAWERTKISDCVADYTVLYNSTSFSTFKCYGVSPS |
| | | | KLIDLCFTSVYADTFLIRSSEVRQVAPGETGVIADYNYKLPDDFTGCVIAWNTAKQDQGQYYYRSHRKTK |
| | | | LKPFERDLSSDENGVRTLSTYDFYPSVPVAYQATRVVVLSFELLNAPATVCGPKLSTQLVKNQCVNFNFN |
| | | | GLKGTGVLTESSKRFQSFQQFGRDTSDFTDSVRDPQTLEILDISPCSFGG |
| Q0Q475 | 307-566 | 17 | SNFRVTPTQEVVRFPNITNRCPFDKVFNASRFPNVYAWERTKISDCVADYTVLYNSTSFSTFKCYGVSPS |
| | | | KLIDLCFTSVYADTFLIRSSEVRQVAPGETGVIADYNYKLPDDFTGCVIAWNTAQQDQGQYYYRSYRKEK |
| | | | LKPFERDLSSDENGVYTLSTYDFYPSIPVEYQATRVVVLSFELLNAPATVCGPKLSTQLVKNQCVNFNFN |
| | | | GLRGTGVLTTSSKRFQSFQQFGRDTSDFTDSVRDPQTLEILDISPCSFGG |
| Q0QDX9 | 307-566 | 18 | SNFRVTPTQEVVRFPNITNRCPFDKVFNASRFPNVYAWERTKISDCVADYTVLYNSTSFSTFKCYGVSPS |
| | | | KLIDLCFTSVYADTFLIRSSEVRQVAPGETGVIADYNYKLPDDFTGCVIAWNTAQQDQGQYYYRSYRKEK |
| | | | LKPFERDLSSDENGVYTLSTYDFYPSIPVEYQATRVVVLSFELLNAPATVCGPKLSTQLVKNQCVNFNFN |
| | | | GLRGTGVLTTSSKRFQSFQQFGRDTSDFTDSVRDPQTLEILDISPCSFGG |
| Q3LZX1 | 307-567 | 19 | SNFRVSPTQEVIRFPNITNRCPFDKVFNATRFPNVYAWERTKISDCVADYTVLYNSTSFSTFKCYGVSPS |
| | | | KLIDLCFTSVYADTFLIRSSEVRQVAPGETGVIADYNYKLPDDFTGCVIAWNTAKHDTGNYYYRSHRKTK |
| | | | LKPFERDLSSDDGNGVYTLSTYDFNPNVPVAYQATRVVVLSFELLNAPATVCGPKLSTELVKNQCVNFNF |
| | | | NGLKGTGVLTSSSKRFQSFQQFGRDTSDFTDSVRDPQTLEILDISPCSFGG |
| A0A096XNM6 | 307-567 | 20 | SNFRVSPTQEVIRFPNITNRCPFDKVFNVTRFPNVYAWERTKISDCVADYTVLYNSTSFSTFKCYGVSPS |
| | | | KLIDLCFTSVYADTFLIRSSEVRQVAPGETGVIADYNYKLPDDFTGCVIAWNTAKQDIGNYYYRSHRKTK |
| | | | LKPFERDLSSDDGNGVYTLSTYDFNPNVPVAYQATRVVVLSFELLNAPATVCGPKLSTQLVKNQCVNFNF |
| | | | NGLKGTGVLTSSSKRFQSFQQFGRDTSDFTDSVRDPQTLEILDISPCSFGG |
| A0A2D1PX86 | 307-567 | 21 | SNFRVSPTQEVIRFPNITNRCPFDKVFNASRFPNVYAWERTKISDCVADYTVLYNSTSFSTFKCYGVSPS |
| | | | KLIDLCFTSVYADTFLIRSSEVRQVAPGETGVIADYNYKLPDDFTGCVIAWNTAKQDTGHYYYRSHRKTK |
| | | | LKPFERDLSSDDGNGVYTLSTYDFNPNVPVAYQATRVVVLSFELLNAPATVCGPKLSTQLVKNQCVNFNF |
| | | | NGLKGTGVLTDSSKRFQSFQQFGRDTSDFTDSVRDPQTLEILDITPCSFGG |
| A0A0U1WHJ8 | 307-567 | 22 | SNFRVSPTQEVVRFPNITNRCPFDKVFNATRFPNVYAWERTKISDCVADYTVLYNSTSFSTFKCYGVSPS |
| | | | KLIDLCFTSVYADTFLIRSSEVRQVAPGETGVIADYNYKLPDDFTGCVIAWNTAKQDTGNYYYRSHRKTK |
| | | | LKPFERDLSSDDGNGVYTLSTYDFNPNVPVAYQATRVVVLSFELLNAPATVCGPKLSTQLVKNQCVNFNF |
| | | | NGLKGTGVLTPSLKRFQSFQQFGRDTSDFTDSVRDPQTLEILDISPCSFGG |
| D5HJU5 | 307-567 | 23 | SNFRVSPTQEVIRFPNITNRCPFDRVFNASRFPSVYAWERTKISECVADYTVLYNSTSFSTFKCYGVSPS |
| | | | KLIDLCFTSVYADTFLIRSSEVRQVAPGETGVIADYNYKLPDDFTGCVIAWNTAKQDTGNYYYRSHRKTK |
| | | | LKPFERDLSSDDGNGVYTLSTYDFNPNVPVAYQATRVVVLSFELLNAPATVCGPKLSTQLVKNQCVNFNF |
| | | | NGLKGTGVLTPSSKRFQSFQQFGRDTSDFTDSVRDPQTLEILDISPCSFGG |
| A0A0U1WHI2 | 306-566 | 24 | SNFRVTPTQEVVRFPNITNRCPFDRVFNASRFPSVYAWERTKISDCVADYTVLYNSTSFSTFKCYGVSPS |
| | | | KLIDLCFTSVYADTFLIRSSEVRQVAPGETGVIADYNYKLPDDFTGCVIAWNTAKQDTGYYYYRSHRKTK |
| | | | LKPFERDLSSDDGNGVYTLSTYDFNPNVPVAYQATRVVVLSFELLNAPATVCGPKLSTELVKNQCVNFNF |
| | | | NGLKGTGVLTKSSKRFQSFQQFGRDTSDFTDSVRDPQTLEILDISPCSFGG |
| R9QTA0 | 306-565 | 25 | SNFRVSPTQEVVRFPNITNRCPFDKVFNATRFPSVYAWERTKISDCVADYTVLYNSTSFSTFKCYGVSPS |
| | | | KLIDLCFTSVYADTFLIRSSEVRQVAPGETGVIADYNYKLPDDFTGCVIAWNTANQDQGQYYYRSSRKEK |
| | | | LKPFERDLSSDENGVYTLSTYDFYPSVPLDYQATRVVVLSFELLNAPATVCGPKLSTTLVKNQCVNFNFN |
| | | | GLKGTGVLTASSKKFQSFQQFGRDASDFTDSVRDPQTLEILDISPCSFGG |
| R9QTH3 | 307-566 | 26 | SNFRVSPSTEVIRFPNITNRCPFDRVFNASRFPSVYAWERTKISDCVADYTVLYNSTSFSTFKCYGVSPS |
| | | | KLIDLCFTSVYADTFLIRFSEVRQIAPGETGVIADYNYKLPDEFTGCVIAWNTANQDRGQYYYRSSRKTK |
| | | | LKPFERDLSSDENGVRTLSTYDFYPSVPLEYQATRVVVLSFELLNAPATVCGPKLSTSLIKNQCVNFNFN |
| | | | GLKGTGVLTDSSKKFQSFQQFGRDASDFTDSVRDPQTLQILDISPCSFGG |
| A0A1W5YKT9 | 299-558 | 27 | SNFRVSPTREVVRFPNITNRCPFDSIFNASRFPSVYAWERTKISDCVADYTVLYNSTSFSTFKCYGVSPS |
| | | | KLIDLCFTSVYADTFLIRFSEVRQVAPGETGVIADYNYRLPDDFTGCVIAWNTANQDVGSYFYRSHRSTK |
| | | | LKPFERDLSSDENGVRTLSTYDFNPYVPLDYQATRVVVLSFELLNAPATVCGPKLSTELVKNQCVNFNFN |
| | | | GLKGTGVLSSSSKRFQSFQQFGRDASDFTDSVRDPQTLEILDITPCSFGG |
| A0A0U1WJY8 | 299-558 | 28 | SNFRVSPTREVVRFPNITNRCPFDSIFNASRFPSVYAWERTKISDCVADYTVLYNSTLFSTFKCYGVSPS |
| | | | KLIDLCFTSVYADTFLIRFSEVRQVAPGETGVIADYNYRLPDDFTGCVIAWNTANQDVGSYFYRSHRSTK |
| | | | LKPFERDLSSDENGVRTLSTYDFNPNVPLDYQATRVVVLSFELLNAPATVCGPKLSTELVKNQCVNFNFN |
| | | | GLKGTGVLTSSSKRFQSFQQFGRDASDFTDSVRDPQTLEILDITPCSFGG |
| A0A2D1PX37 | 300-559 | 29 | SNFRVQPTVDVVRFPNITNLCPFDAVFNATRFPSVYAWERVKISNCVADYTAFYNSTSFSTFKCYGVSPS |
| | | | KLIDLCFTSVYADTFLIRFSEVRQVAPGETGVIADYNYKLPDDFTGCVIAWNTAKQDVGSYFYRSHRSSK |
| | | | LKPFERDLSSDENGVRTLSTYDFNPNVPLDYQATRVVVLSFELLNAPATVCGPKLSTQLVKNQCVNFNFN |
| | | | GLKGTGVLTDSSKRFQSFQQFGRDTSDFTDSVRDPQTLDILDITPCSFGG |
| Q0QDZ0 | 307-566 | 30 | SNFRVSPVTEVVRFPNITNLCPFDKVFNATRFPSVYAWERTKISDCVADYTVFYNSTSFSTFNCYGVSPS |
| | | | KLIDLCFTSVYADTFLIRFSEVRQVAPGQTGVIADYNYKLPDDFTGCVIAWNTAKQDVGSYFYRSHRSSK |
| | | | LKPFERDLSSEENGVRTLSTYDFNQNVPLEYQATRVVVLSFELLNAPATVCGPKLSTSLVKNQCVNFNFN |
| | | | GFKGTGVLTDSSKTFQSFQQFGRDASDFTDSVRDPQTLRILDISPCSFGG |
| A0A0U1WHH0 | 307-566 | 31 | SNFRVSPVTEVVRFPNITNLCPFDKVFNATRFPSVYAWERTKISDCVADYTVFYNSTSFSTFNCYGVSPS |
| | | | KLIDLCFTSVYADTFLIRFSEVRQVAPGQTGVIADYNYKLPDDFTGCVIAWNTAKQDVGSYFYRSHRSSK |
| | | | LKPFERDLSSEENGVRTLSTYDFNQYVPLEYQATRVVVLSFELLNAPATVCGPKLSTSLVKNQCVNFNFN |
| | | | GFKGTGVLTDSSKTFQSFQQFGRDASDFTDSVRDPQTLRILDISPCSFGG |
| Q0Q484 | 307-566 | 32 | SNFRVSPVTEVVRFPNITNLCPFDKVFNATRFPSVYAWERTKISDCVADYTVFYNSTSFSTFNCYGVSPS |
| | | | KLIDLCFTSVYADTFLIRFSEVRQVAPGQTGVIADYNYKLPDDFTGCVIAWNTAKQDVGSYFYRSHRSSK |
| | | | LKPFERDLSSVEENGRTLSTYDFNQNVPLEYQATRVVVLSFELLNAPATVCGPKLSTSLVKNQCVNFNFN |
| | | | GFKGTGVLTDSSKTFQSFQQFGRDASDFTDSVRDPQTLRILDISPCSFGG |
| A0A0K1Z074 | 307-566 | 33 | SNFRVAPVTEVVRFPNITNLCPFDKVFNATRFPSVYAWERTKISDCVADYTVFYNSTSFSTFNCYGVSPS |
| | | | KLIDLCFTSVYADTFLIRFSEVRQVAPGQTGVIADYNYKLPDDFTGCVIAWNTAKYDVGSYFYRSHRSSK |
| | | | LKPFERDLSSEENGARTLSTYDFNQNVPLEYQATRVVVLSFELLNAPATVCGPKLSTSLVKNQCVNFNFN |
| | | | GFKGTGVLTDSSKTFQSFQQFGRDASDFTDSVRDPKTLQILDISPCSFGG |
| A0A0U1UYX4 | 302-561 | 34 | SNFRVQPTVDVARFPNITNVCPFDKVFNATRFPSVYAWERTKISDCVADYTVFYNSTSFSTFNCYGVSPS |
| | | | KLIDLCFTSVYADTFLIRFSEVRQVAPGQTGVIADYNYKLPDDFIGCVIAWNTAKQDVGSYFYRSHRSSK |
| | | | LKPFERDLSSEENGVLTLSTYDFNQNVPLEYQATRVVVLSFELLNAPATVCGPKLSTPLVKNQCVNFNFN |
| | | | GLKGTGVLTDSSKTFQSFQQFGRDASDFTDSVRDPQTLQILDISPCSFGG |
| A0A4Y6GL43 | 306-566 | 35 | SNFRVSPTQEVVRFPNITNRCPFDKVFNASRFPSVYAWERIKISDCVADYTVLYNSTSFSTFKCYGVSPS |
| | | | KLIDLCFTSVYADTFLIRSSEVRQVAPGETGVIADYNYKLPDDFTGCVIAWNTAKQDTGSYYYRSHRKTK |
| | | | LKPFERDLSSDDGNGVYTLSTYDFNPNVPVAYQATRVVVLSFELLNAPATVCGPKLSTQLVKNQCVNFNF |
| | | | NGLTGTGVLTPSSKRFQPFQQFGRDVSDFTDSVRDPKTSEILDISPCSFGG |
| A3EXG6 | 338-585 | 36 | SRYRAQVAGFVRVTQRGSYCTPPYSVLQDPPQPVVWRRYMLYDCVFDFTVVVDSLPTHQLQCYGVSPRRL |
| | | | ASMCYGSVTLDVMRINETHLNNLFNRVPDTFSLYNYALPDNFYGCLHAFYLNSTAPYAVANRFPIKPGGR |
| | | | QSNSAFIDTVINAAHYSPFSYVYGLAVITLKPAAGSKLVCPVANDTVVITDRCVQYNLYGYTGTGVLSKN |
| | | | TSLVIPDGKVFTASSTGTIIGVSINSTTYSIMPCVTVP |
| P36334 | 315-678 | 37 | NGYTVQPIADVYRRKPNLPNCNIEAWLNDKSVPSPLNWERKTFSNCNFNMSSLMSFIQADSFTCNNIDAA |
| | | | KIYGMCFSSITIDKFAIPNGRKVDLQLGNLGYLQSFNYRIDTTATSCQLYYNLPAANVSVSRFNPSTWNK |
| | | | RFGFIEDSVFKPRPAGVLTNHDVVYAQHCFKAPKNFCPCKLNGSCVGSGPGKNNGIGTCPAGTNYLTCDN |
| | | | LCTPDPITFTGTYKCPQTKSLVGIGEHCSGLAVKSDYCGGNSCTCRPQAFLGWSADSCLQGDKCNIFANF |
| | | | ILHDVNSGLTCSTDLQKANTDIILGVCVNYDLYGILGQGIFVEVNATYYNSWQNLLYDSNGNLYGFRDYI |
| | | | INRTFMIRSCYSGR |
| P25194 | 311-688 | 38 | NGYTVQPIADVYRRIPNLPDCNIEAWLNDKSVPSPLNWERKTFSNCNFNMSSLMSFIQADSFTCNNIEAA |
| | | | KIYGMCFSSITIDKFAIPNGRKVDLQLGNLGYLQSFNYRIDTTAASCQLYYNLPAANVSVSRFNPSTWNR |
| | | | RFGFTEQSVFKPQPVGVFTHHDVVYAQHCFKAPTNFCPCKLDGSLCVGNGPGIDAGYKNSGIGTCPAGTN |
| | | | YLTCHNAAQCDCLCTPDPITSKSTGPYKCPQTKYLVGIGEHCSGLAIKSDYCGGNPCTCQPQAFLGWSVD |
| | | | SCLQGDRCNIFANFILHDVNSGTTCSTDLQKSNTDIILGVCVNYDLYGITGQGIFVEVNATYYNSWQNLL |
| | | | YDSNGNLYGFRDYLTNRTFMIRSCYSGR |
| Q8JSP8 | 311-674 | 39 | NGYTVQPVATVYRRIPDLPNCDIEAWLNSKTVSSPLNWERKIFSNCNFNMGRLMSFIQADSFGCNNIDAS |
| | | | RLYGMCFGSITIDKFAIPNSRKVDLQVGKSGYLQSFNYKIDTAVSSCQLYYSLPAANVSVTHYNPSSWNR |
| | | | RYGFINQSFGSRGLHDAVYSQQCFNTPNTYCPCRTSQCIGGAGTGTCPVGTTVRKCFAAVTNATKCTCWC |
| | | | QPDPSTYKGVNAWTCPQSKVSIQPGQHCPGLGLVEDDCSGNPCTCKPQAFIGWSSETCLQNGRCNIFANF |
| | | | ILNDVNSGTTCSTDLQQGNTNITTDVCVNYDLYGITGQGILIEVNATYYNSWQNLLYDSSGNLYGFRDYL |
| | | | SNRTFLIRSCYSGR |
| Q9IKD1 | 309-676 | 40 | SGYTVQPVGLVYRRVRNLPDCKIEEWLAANTVPSPLNWERKTFQNCNFNLSSLLRFVQAESLSCSNIDAS |
| | | | KVYGMCFGSISIDKFAIPNSRRVDLQLGKSGLLQSFNYKIDTRATSCQLYYSLAQDNVTVINHNPSSWNR |
| | | | RYGFNDVATFHSGEHDVAYAEACFTVGASYCPCAKPSTVYSCVTGKPKSANCPTGTSNRECNVQASGFKS |
| | | | KCDCTCNPSPLTTYDPRCLQARSMLGVGDHCEGLGILEDKCGGSNICNCSADAFVGWAMDSCLSNARCHI |
| | | | FSNLMLNGINSGTTCSTDFQLPNTEVVTGVCVKYDLYGSTGQGVFKEVKADYYNSWQNLLYDVNGNLNGF |
| | | | RDIVTNKTYLLRSCYSGR |
| Q5MQD0 | 307-678 | 41 | SGFTVKPVATVHRRIPDLPDCDIDKWLNNFNVPSPLNWERKIFSNCNFNLSTLLRLVHTDSFSCNNFDES |
| | | | KIYGSCFKSIVLDKFAIPNSRRSDLQLGSSGFLQSSNYKIDTTSSSCQLYYSLPAINVTINNYNPSSWNR |
| | | | RYGFNNFNLSSHSVVYSRYCFSVNNTFCPCAKPSFASSCKSHKPPSASCPIGTNYRSCESTTVLDHTDWC |
| | | | RCSCLPDPITAYDPRSCSQKKSLVGVGEHCAGFGVDEEKCGVLDGSYNVSCLCSTDAFLGWSYDTCVSNN |
| | | | RCNIFSNFILNGINSGTTCSNDLLQPNTEVFTDVCVDYDLYGITGQGIFKEVSAVYYNSWQNLLYDSNGN |
| | | | IIGFKDFVTNKTYNIFPCYAGR |
| Q0ZME7 | 307-676 | 42 | SGFTVKPVATVYRRIPNLPDCDIDNWLNNVSVPSPLNWERRIFSNCNFNLSTLLRLVHVDSFSCNNLDKS |
| | | | KIFGSCFNSITVDKFAIPNRRRDDLQLGSSGFLQSSNYKIDISSSSCQLYYSLPLVNVTINNFNPSSWNR |
| | | | RYGFGSFNLSSYDVVYSDHCFSVNSDFCPCADPSVVNSCAKSKPPSAICPAGTKYRHCDLDTTLYVKNWC |
| | | | RCSCLPDPISTYSPNTCPQKKVVVGIGEHCPGLGINEEKCGTQLNHSSCFCSPDAFLGWSFDSCISNNRC |
| | | | NIFSNFIFNGINSGTTCSNDLLYSNTEISTGVCVNYDLYGITGQGIFKEVSAAYYNNWQNLLYDSNGNII |
| | | | GFKDFLTNKTYTILPCYSGR |
| P11224 | 309-637 | 43 | SGYTVQPVGVVYRRVANLPACNIEEWLTARSVPSPLNWERKTFQNCNFNLSSLLRYVQAESLFCNNIDAS |
| | | | KVYGRCFGSISVDKFAVPRSRQVDLQLGNSGFLQTANYKIDTAATSCQLHYTLPKNNVTINNHNPSSWNR |
| | | | RYGFNDAGVFGKNQHDVVYAQQCFTVRSSYCPCAQPDIVSPCTTQTKPKSAFVNVGDHCEGLGVLEDNCG |
| | | | NADPHKGCICANNSFIGWSHDTCLVNDRCQIFANILLNGINSGTTCSTDLQLPNTEVVTGICVKYDLYGI |
| | | | TGQGVFKEVKADYYNSWQTLLYDVNGNLNGFRDLTINKTYTIRSCYSGR |
| P11225 | 309-548 | 44 | SGYTVQPVGVVYRRVPNLPDCKIEEWLTAKSVPSPLNWERRTFQNCNFNLSSLLRYVQAESLSCNNIDAS |
| | | | KVYGMCFGSVSVDKFAIPRSRQIDLQIGNSGFLQTANYKIDTAATSCQLYYSLPKNNVTINNYNPSSWNR |
| | | | RYGFKVNDRCQIFANILLNGINSGTTCSTDLQLPNTEVATGVCVRYDLYGITGQGVFKEVKADYYNSWQA |
| | | | LLYDVNGNLNGFRDLTINKTYTIRSCYSGR |
| Q6Q1S2 | 462-682 | 45 | NFLDDNVLPETYVALPIYYQHTDINFTATASFGGSCYVCKPHQVNISLNGNTSVCVRTSHFSIRYIYNRV |
| | | | KSGSPGDSSWHIYLKSGTCPFSFSKLNNFQKFKTICFSTVEVPGSCNFPLEATWHYTSYTIVGALYVTWS |
| | | | EGNSITGVPYPVSGIREFSNLVLNNCTKYNIYDYVGTGIIRSSNQSLAGGITYVSNSGNLLGFKNVSTGN |
| | | | IFIVTPCNQPD |
| P15423 | 279-501 | 46 | SPIQSVELPVSIVSLPVYHKHTFIVLYVDFKPQSGGGKCFNCYPAGVNITLANFNETKGPLCVDTSHFTT |
| | | | KYVAVYANVGRWSASINTGNCPFSFGKVNNFVKFGSVCFSLKDIPGGCAMPIVANWAYSKYYTIGSLYVS |
| | | | WSDGDGITGVPQPVEGVSSFMNVTLDKCTKYNIYDVSGVGVIRVSNDTFLNGITYTSTSGNLLGFKDVTK |
| | | | GTIYSITPCNPPD |
| A3EXD0 | 203-487 | 47 | CAGETNFKSLSLWDTPASDCVSGSYNQEATLGAFKVYFDLINCTFRYNYTITEDENAEWFGITQDTQGVH |
| | | | LYSSRKENVFRNNMFHFATLPVYQKILYYTVIPRSIRSPFNDRKAWAAFYIYKLHPLTYLLNFDVEGYIT |
| | | | KAVDCGYDDLAQLQCSYESFEVETGVYSVSSFEASPRGEFIEQATTQECDFTPMLTGTPPPIYNFKRLVF |
| | | | TNCNYNLTKLLSLFQVSEFSCHQVSPSSLATGCYSSLTVDYFAYSTDMSSYLQPGSAGAIVQFNYKQDFS |
| | | | NPTCR |
| A3EX94 | 369-663 | 48 | SSYEASATGTFIEQPNATECDFSPMLTGVAPQVYNFKRLVFSNCNYNLTKLLSLFAVDEFSCNGISPDSI |
| | | | ARGCYSTLTVDYFAYPLSMKSYIRPGSAGNIPLYNYKQSFANPTCRVMASVLANVTITKPHAYGYISKCS |
| | | | RLTGANQDVETPLYINPGEYSICRDFSPGGFSEDGQVFKRTLTQFEGGGLLIGVGTRVPMTDNLQMSFII |
| | | | SVQYGTGTDSVCPMLDLGDSLTITNRLGKCVDYSLYGVTGRGVFQNCTAVGVKQQRFVYDSFDNLVGYYS |
| | | | DDGNYYCVRPCVSVP |
| K9N5Q8 | 203-497 | 49 | SFATYHTPATDCSDGNYNRNASLNSFKEYFNLRNCTFMYTYNITEDEILEWFGITQTAQGVHLFSSRYVD |
| | | | LYGGNMFQFATLPVYDTIKYYSIIPHSIRSIQSDRKAWAAFYVYKLQPLTFLLDFSVDGYIRRAIDCGFN |
| | | | DLSQLHCSYESFDVESGVYSVSSFEAKPSGSVVEQAEGVECDFSPLLSGTPPQVYNFKRLVFTNCNYNLT |
| | | | KLLSLFSVNDFTCSQISPAAIASNCYSSLILDYFSYPLSMKSDLSVSSAGPISQFNYKQSFSNPTCLILA |
| | | | TVPHNLTTITKPLKY |
| A0A3Q8AKM0 | 306-583 | 50 | SNFRVSPSTEVVRFPNITNLCPFGQVFNASNFPSVYAWERLRISDCVADYAVLYNSSSSFSTFKCYGVSP |
| | | | TKLNDLCFSSVYADYFVVKGDDVRQIAPAQTGVIADYNYKLPDDFTGCVLAWNTNSVDSKSGNNFYYRLF |
| | | | RHGKIKPYERDISNVLYNSAGGTCSSISQLGCYEPLKSYGFTPTVGVGYQPYRVVVLSFELLNAPATVCG |
| | | | PKKSTELVKNKCVNFNFNGLTGTGVLTSSTKKFQPFQQFGRDVSDFTDSVRDPKTFEILDISPCSYGG |
| E0XIZ3 | 307-581 | 51 | SNFRVTPTTEVVRFPNITQLCPFNEVFNITSFPSVYAWERMRITNCVADYSVLYNSSASFSTFQCYGVSP |
| | | | TKLNDLCFSSVYADYFVVKGDDVRQIAPAQTGVIADYNYKLPDDFTGCVIAWNTNSLDSSNEFFYRRFRH |
| | | | GKIKPYGRDLSNVLFNPSGGTCSAEGLNCYKPLASYGFTQSSGIGFQPYRVVVLSFELLNAPATVCGPKQ |
| | | | STELVKNKCVNFNFNGLTGTGVLTNSTKKFQPFQQFGRDVSDFTDSVRDPKTLEILDIAPCSYGG |
| A0A2D1PXA9 | 303-580 | 52 | SNFRVAPSKEVVRFPNITNLCPFGEVfNATTFPSVYAWERKRISNCVADYSVLYNSTSFSTFKCYGVSAT |
| | | | KLNDLCFSNVYADSFVVKGDDVRQIAPGQTGVIADYNYKLPDDFLGCVLAWNTNSKDSSTSGNYNYLYRW |
| | | | VRRSKLNPYERDLSNDIYSPGGQSCSAIGPNCYNPLRPYGFFTTAGVGHQPYRVVVLSFELLNAPATVCG |
| | | | PKLSTDLIKNQCVNFNFNGLTGTGVLTSSSKRFQPFQQFGRDVSDFTDSVRDPKTSEILDISPCSFGG |
| U5WLK5 | 304-581 | 53 | SNFRVAPSKEVVRFPNITNLCPFGEVFNATTFPSVYAWERKRISNCVADYSVLYNSTSFSTFKCYGVSAT |
| | | | KLNDLCFSNVYADSFVVKGDDVRQIAPGQTGVIADYNYKLPDDFLGCVLAWNTNSKDSSTSGNYNYLYRW |
| | | | VRRSKLNPYERDLSNDIYSPGGQSCSAVGPNCYNPLRPYGFFTTAGVGHQPYRVVVLSFELLNAPATVCG |
| | | | PKLSTDLIKNQCVNFNFNGLTGTGVLTPSSKRFQPFQQFGRDVSDFTDSVRDPKTSEILDISPCSFGG |
| A0A2D1PX29 | 304-581 | 54 | SNFRVAPSKEVVRFPNITNLCPFGEVFNATTFPSVYAWERKRISNCVADYSILYNSTSFSTFKCYGVSAT |
| | | | KLNDLCFSNVYADSFVVKGDDVRQIAPGQTGVIADYNYKLPDDFLGCVLAWNTNSKDSSTSGNYNYLYRW |
| | | | VRRSKLNPYERDLSNDIYSPGGQSCSAVGPNCYNPLRPYGFFTTAGVGHQPYRVVVLSFELLNAPATVCG |
| | | | PKLSTDLIKNQCVNFNFNGLTGTGVLTPSSKRFQPFQQFGRDVSDFTDSVRDPKTSEILDISPCSFGG |
| A0A2D1PX97 | 303-580 | 55 | SNFRVAPSKEVVRFPNITNLCPFGEVFNATTFPSVYAWERKRISNCVADYSVLYNSTSFSTFKCYGVSAT |
| | | | KLNDLCFSNVYADSFVVKGDDVRQIAPGQTGVIADYNYKLPDDFTGCVLAWNTRNIDATQTGNYNYKYRS |
| | | | LRHGKLRPFERDISNVPFSPDGKPCTPPAFNCYWPLNDYGFYITNGIGYQPYRVVVLSFELLNAPATVCG |
| | | | PKLSTDLIKNQCVNFNFNGLTGTGVLTPSSKRFQPFQQFGRDVSDFTDSVRDPKTSEILDISPCSFGG |
| U5WHZ7 | 304-581 | 56 | SNFRVAPSKEVVRFPNITNLCPFGEVFNATTFPSVYAWERKRISNCVADYSVLYNSTSFSTFKCYGVSAT |
| | | | KLNDLCFSNVYADSFVVKGDDVRQIAPGQTGVIADYNYKLPDDFTGCVLAWNTRNIDATQTGNYNYKYRS |
| | | | LRHGKLRPFERDISNVPFSPDGKPCTPPAFNCYWPLNDYGFYITNGIGYQPYRVVVLSFELLNAPATVCG |
| | | | PKLSTDLIKNQCVNFNFNGLTGTGVLTPSSKRFQPFQQFGRDVSDFTDSVRDPKTSEILDISPCSFGG |
| U5WI05 | 304-581 | 57 | SNFRVAPSKEVVRFPNITNLCPFGEVFNATTFPSVYAWERKRISNCVADYSVLYNSTSFSTFKCYGVSAT |
| | | | KLNDLCFSNVYADSFVVKGDDVRQIAPGQTGVIADYNYKLPDDFTGCVLAWNTRNIDATQTGNYNYKYRS |
| | | | LRHGKLRPFERDISNVPFSPDGKPCTPPAFNCYWPLNDYGFYITNGIGYQPYRVVVLSFELLNAPATVCG |
| | | | PKLSTDLIKNQCVNFNFNGLTGTGVLTPSSKRFQPFQQFGRDVSDFTDSVRDPKTSEILDISPCSFGG |
| A0A023PUW9 | 307-584 | 58 | SNFRVSPSKEVVRFPNITNLCPFGEVFNATTFPSVYAWERKRISNCVADYSVLYNSTSFSTFKCYGVSAI |
| | | | KLNDLCFSNVYADSFVVKGDDVRQIAPGQTGVIADYNYKLPDDFMGCVLAWNTRNIDATSSGNFNYKYRS |
| | | | LRHGKLRPFERDISNVPFSPDGKPCTPPAFNCYWPLNDYGFYTTNGIGYQPYRVVVLSFELLNAPATVCG |
| | | | PKLSTDLITNQCVNFNFNGLTGTGVLTPSLKRFQPFQQFGRDFSDFTDSVRDPKTLEVLDISPCSFGG |
| A0A023PTS3 | 307-584 | 59 | SNFRVSPSREVVRFPNITNLCPFGEVFNATTFPSVYAWERKRISNCVADYSVLYNSTSFSTFKCYGVSAI |
| | | | KLNDLCFSNVYADSFVVKGDDVRQIAPGQTGVIADYNYKLPDDFMGCVLAWNTRNIDATSSGNFHYKYRS |
| | | | LRHGKLRPFERDISNVPFSPDGKPCTPPAFNCYWPLNDYGFYTTNGIGYQPYRVVVLSFELLNAPATVCG |
| | | | PKLSTDLITNQCVNFNFNGLTGTGVLTPSSKRFQPFQQFGRDVSDFTDSVRDPKTLEVLDISPCSFGG |
| A0A2D1PXC0 | 304-581 | 60 | SNFRVAPSKEVVRFPNITNLCPFGEVFNATTFPSVYAWERKRISNCVADYSVLYNSTSFSTFKCYGVSAT |
| | | | KLNDLCFSNVYADSFVVKGDDVRQIAPGQTGVIADYNYKLPDDFMGCVLAWNTRNIDATSTGNYNYKYRS |
| | | | LRHGKLRPFERDISNVPFSPDGKPCTPPAFNCYWPLNDYGFFTTNGIGYQPYRVVVLSFELLNAPATVCG |
| | | | PKLSTDLIKNQCVNFNFNGLTGTGVLTSSSKRFQPFQQFGRDVSDFTDSVRDPKTSEILDISPCSFGG |
| Q6TPE8 | 303-580 | 61 | SNFRVVPSRDVVRFPNITNLCPFGEVFNATKFPSVYAWERKRISNCVADYSVLYNSTFFSTFKCYGVSAT |
| | | | KLNDLCFSNVYADSFVVKGDDVRQIAPGQTGVIADYNYKLPDDFMGCVLAWNTRNIDATSTGNYNYKYRY |
| | | | LRHGKLRPFERDISNVPFSPDGKPCTPPALNCYWPLNDYGFYTTTGIGYQPYRVVVLSFELLNAPATVCG |
| | | | PKLSTDLIKNQCVNFNFNGLTGTGVLTPSSKRFQPFQQFGRDVSDFTDSVRDPKTSEILDISPCSFGG |
| Q1T6X6 | 303-580 | 62 | SNFRVVPSGDVVRFPNITNLCPFGEVFNATKFPSVYAWERKKISNCVADYSVLYNSTFFSTFKCYGVSAT |
| | | | KLNDLCFSNVYADSFVVKGDDVRQIAPGQTGVIADYNYKLPDDFMGCVLAWNTRNIDATSTGNYNYKYRC |
| | | | LRHGKLRPFERDISNVPFSPDGKPCTPPAFNCYWPLNDYGFYTTTGIGYQPYRVVVLSFELLNAPATVCG |
| | | | PKLSTDLIKNQCVNFNFNGLTGTGVLTPSSKRFQPFQQFGRDVSDFTDSVRDPKTSEILDISPCSFGG |
| D2E1D2 | 303-580 | 63 | SNFRVVPSGDVVRFPNITNLCPFGEVFNATKFPSVYAWERKKISNCVADYSVLYNSTFFSTFKCYGVSAT |
| | | | KLNDLCFSNVYADSFVVKGDDVRQIAPGQTGVIADYNYKLPDDFMGCVLAWNTRNIDATSTGNYNYKYRY |
| | | | LRHGKLRPFERDISNVPFSPDGKPCTPPALNCYWPLNDYGFYTTTGIGYQPYRVVVLSFELLNAPATVCG |
| | | | PKLSTDLIKNQCVNFNFNGLTGTGVLTPSSKRFQPFQQFGRDVSDFTDSVRDPKTSEILDISPCSFGG |
| Q6DSU4 | 303-580 | 64 | SNFRVVPSGDVVRFPNITNLCPFGEVFNATKFPSVYAWERKKISNCVADYSVLYNSTFFSTFKCYGVSAT |
| | | | KLNDLCFSNVYADSFVVKGDDVRQIAPGQTGVIADYNYKLPDDFMGCVLAWNTRNIDATSTGNYNYKYRY |
| | | | LKHGKLRPFERDISNVPFSPDGKPCTPPALNCYWPLNDYGFYTTTGIGYQPYRVVVLSFELLNAPATVCG |
| | | | PKLSTDLIKNQCVNFNFNGLTGTGVLTPSSKRFQPFQQFGRDVSDFTDSVRDPKTSEILDISPCSFGG |
| Q202H8 | 303-580 | 65 | SNFRVVPSGDVVRFPNITNLCPFGEVFNATKFPSVYAWERKKISNCVADYSVLYNSTFFSTFKCYGVSAT |
| | | | KLNDLCFSNVYVDSFVVKGDDVRQIAPGQTGVIADYNYKLPDDFMGCVLAWNTRNIDATSTGNYNYKYRY |
| | | | LRHGKLRPFERDISNVPFSPDGKPCTPPALNCYWPLNDYGFYTTTGIGYQPYRVVVLSFELLNAPATVCG |
| | | | PKLSTDLIKNQCVNFNFNGLTGTGVLTPSSKRFQPFQQFGRDVSDFTDSVRDPKTSEILDISPCSFGG |
| Q202F2 | 303-580 | 66 | SNFRVVPSGDVVRFPNITNLCPFGEVFNATKFPSVYAWERKKISNCVVDYSVLYNSTFFSTFKCYGVSAT |
| | | | KLNDLCFSNVYADSFVVKGDDVRQIAPGQTGVIADYNYKLPDDFMGCVLAWNTRNIDATSTGNYNYKYRY |
| | | | LRHGKLRPFERDISNVPFSPDGKPCTPPALNCYWPLNDYGFYTTTGIGYQPYRVVVLSFELLNAPATVCG |
| | | | PKLSTDLIKNQCVNFNFNGLTGTGVLTPSSKRFQPFQQFGRDVSDFTDSVRDPKTSEILDISPCSFGG |
| D2E235 | 303-580 | 67 | SNFRVVPSGDVVRFPNITNLCPFGEVFNATKFPSVYAWERKKISNCVADYSVLYNSTFFSTFKCYGVSAT |
| | | | KLNDLCFSNVYADSFVVKGDDVRQIAPGQTGVIADYNYKLPDDFMGCVLAWNTRNIDATSTGNHNYKYRY |
| | | | LRHGKLRPFERDISNVPFSPDGKPCTPPALNCYWPLNDYGFYTTTGIGYQPYRVVVLSFELLNAPATVCG |
| | | | PKLSTDLIKNQCVNFNFNGLTGTGVLTPSSKRFQPFQQFGRDVSDFTDSVRDPKTSEILDISPCSFGG |
| Q202H5 | 303-580 | 68 | SNFRVVPSGDVVRFPNITNLCPFGEVFNATKFPSVYAWERKKISNCVADYSVLYNSTFFSTFKCYGVSAT |
| | | | KLNDLCFSNVYADSFVVKGDDVRQIAPGQTGVIADYNYKLPDDFMGCVLAWNTRNIDATSTGNYNYKYRY |
| | | | LRHGKLRPFERDISNVPFSPNGKPCTPPALNCYWPLNDYGFYTTTGIGYQPYRVVVLSFELLNAPATVCG |
| | | | PKLSTDLIKNQCVNFNFNGLTGTGVLTPSSKRFQPFQQFGRDVSDFTDSVRDPKTSEILDISPCSFGG |
| A4ZF30 | 303-580 | 69 | SNFRVVPSGDVVRFPNITNLCPFGEVFNATKFPSVYAWERKKISNCVADYSVLYNSTFFSTFKCYGVSAT |
| | | | KLNDLCFSNVYADSFVVKGDDVRQIAPGQTGVIADYNYKLPDDFMGCVLAWNTRNIDATSTGNYNYKYRY |
| | | | LRHGKLRPFERDISNVPFSPDGKPCTPPAPNCYWPLNDYGFYTTSGIGYQPYRVVVLSFELLNAPATVCG |
| | | | PKLSTDLIKNQCVNFNFNGLTGTGVLTPSSKRFQPFQQFGRDVSDFTDSVRDPKTSEILDISPCAFGG |
| A4ZF29 | 303-580 | 70 | SNFRVVPSGDVVRFPNITNLCPFGEVFNATKFPSVYAWERKKISNCVADYSVLYNSTFFSTFKCYGVSAT |
| | | | KLNDLCFSNVYADSFVVKGDDVRQIAPGQTGVIADYNYKLPDDFMGCVLAWNTRNIDATSTGNYNYKYRY |
| | | | LRHGKLRPFERDISNVPFSPDGKPCTPPAPNCYWPLNGYGFYTTSGIGYQPYRVVVLSFELLNAPATVCG |
| | | | PKLSTDLIKNQCVNFNFNGLTGTGVLTPSSKRFQPFQQFGRDVSDFTDSVRDPKTSEILDISPCSFGG |
| Q4JDP0 | 303-580 | 71 | SNFRVVPSGDVVRFPNITNLCPFGEVFNATKFPSVYAWERKRISNCVADYSVLYNSTSFSTFKCYGVSAT |
| | | | KLNDLCFSNVYADSFVVKGDDVRQIAPGQTGVIADYNYKLPDDFMGCVLAWNTRNIDATSTGNYNYKYRY |
| | | | LRHGKLRPFERDISNVPFSPDGKPCTPPAPNCYWPLNGYGFYTTSGIGYQPYRVVVLSFELLNAPATVCG |
| | | | PKLSTDLIKNQCVNFNFNGLTGTGVLTPSSKRFQPFQQFGRDVSDFTDSVRDPKTSEILDISPCSFGG |
| Q5GDJ7 | 303-580 | 72 | SNFRVVPSGDVVRFPNITNLCPFGEVFNATKFPSVYAWERKRISNCVADYSVLYNSTSFSTFKCYGVSAT |
| | | | KLNDLCFSNVYADSFVVKGDDVRQIAPGQTGVIADYNYKLPDDFMGCVLAWNTRNIDATSTGNYNYKYRY |
| | | | LRHGKLRPFERDISNVPFSPDGKPCTPPAPNCYWPLRGYGFYTTSGIGYQPYRVVVLSFELLNAPATVCG |
| | | | PKLSTDLIKNQCVNFNFNGLTGTGVLTPSSKRFQPFQQFGRDVSDFTDSVRDPKTSEILDISPCSFGG |
| Q3ZTC5 | 303-580 | 73 | SNFRVVPSGDVVRFPNITNLCPFGEVFNATKFPSVYAWERKRISNCVADYSVLYNSTSFSTFKCYGVSAT |
| | | | KLNDLCFSNVYADSFVVKGDDVRQIAPGQTGVIADYNYKLPDDFMGCVLAWNTRNIDATSTGNYNYKYRY |
| | | | LRHGKLRPFERDISNVPFSPDGKPCTPPAPNCYWPLRGYGFYTTSGIGYQPYRVVVLSFELLNAPATVCG |
| | | | PKLSTDLIKNQCVNFNFNGLTGTGVLTPSSKRFQPFQQFGRDVSDFTDSVRDPKTSEILDISPCSFGG |
| Q4JDN4 | 303-580 | 74 | SNFRVVPSGDVVRFPNITNLCPFGEVFNATKFPSVYAWERKRISNCVADYSVLYNSTSFSTFKCYGVSAT |
| | | | KLNDLCFSNVYADSFVVKGDDVRQIAPGQTGVIADYNYKLPDDFMGCVLAWNTRNIDATSTGNYNYKHRY |
| | | | LRHGKLRPFERDISNVPFSPDGKPCTPPAPNCYWPLRGYGFYTTSGIGYQPYRVVVLSFELLNAPATVCG |
| | | | PKLSTDLIKNQCVNFNFNGLTGTGVLTPSSKRFQPFQQFGRDVSDFTDSVRDPKTSEILDISPCSFGG |
| Q3ZTE0 | 303-580 | 75 | SNFRVVPSGDVVRFPNITNLCPFGEVFNATKFPSVYAWERKRISNCVADYSVLYNSTSFSTFKCYGVSAT |
| | | | KLNDLCFSNVYADSFVVKGDDVRQIAPGQTGVIADYNYKLPDDFMGCVLAWNTRNIDATSTGNYNYKYRY |
| | | | LRHGKLRPFERDISNVPFSSDGKPCTPPAPNCYWPLRGYGFYTTSGIGYQPYRVVVLSFELLNAPATVCG |
| | | | PKLSTDLIKNQCVNFNFNGLTGTGVLTPSSKRFQPFQQFGRDVSDFTDSVRDPKTSEILDISPCSFGG |
| Q4JDP2 | 303-580 | 76 | SNFRVVPSGDVVRFPNITNLCPFGEVFNATKFPSVYAWERKRISNCVADYSVLYNSTSFSTFKCYGVSAT |
| | | | KLNDLCFSNVYADSFVVKGDDVRQIAPGQTGVIADYNYKLPDDFMGCVLAWNTRNIDATSTGNYNYKXRY |
| | | | LRHGKLRPFERDISNVPFSPXGKPCTPPAPNCYWPLRGYGFYTTSGIGYQPYRVVVLSFELLNAPATVCG |
| | | | PKLSTDLIKNQCVNFNFNGLTGTGVLTPSSKRFQPFQQFGRDVSDFTDSVRDPKTSEILDISPCSFGG |

### Exemplary SARS-CoV-2 Spike Polypeptide Mutations

In some cases, the SARS-CoV-2 Spike polypeptide fragment is engineered to contain at least one amino acid substitution, such as from 1-10 substitutions, or from 1-5 substitutions, or from 1-3 substitutions, or from 1-2 substitutions in comparison to the amino acid sequence of SEQ ID NO: 1, or in comparison to the Spike polypeptide sequence of a SARS-CoV-2 or coronavirus parental strain. In some cases, the substitutions are intended to increase peptidogenicity of the polypeptide, i.e., a SARS-CoV-2 Spike peptidogenic fragment. In some cases, the substitutions are intended to focus the construct on a particular mutant or strain of SARS-CoV-2, which contains the same or similar substitutions compared to a parental strain.

For example, in some cases, a SARS-CoV-2 Spike polypeptide fragment can be made where one or more amino acid residues are deleted, added, or substituted to generate SARS-CoV-2 peptidogenic proteins having altered conformation dynamics. For example, residues in the hydrophobic “core” of the protein can be substituted with non-polar residues having smaller side chains (supra) in order to create cavities in the core and disrupt the packing, and cysteine residues can be deleted or substituted with other amino acid residues in order to eliminate disulfide bridges (which are often found in protein cores). In some embodiments, at least one disulfide bond is eliminated in the SARS-CoV-2 starting protein, such as, for example, replacing the cysteines with alanines, serines, and/or glycines, etc. In further preferred embodiments, both cysteines involved in the formation of the at least one disulfide bond are replaced with alanines, serines, and/or glycines, or preferably with alanines or glycines, etc.

In some cases, a mutant SARS-CoV-2 Spike polypeptide fragment, such as a SARS-CoV-2 Spike peptidogenic fragment, is tested to confirm that it has a “similar conformation” to the SARS-CoV-2 starting polypeptide fragment on which it is based. Such testing may be performed using a cross-reacting antibody, especially an antibody that recognizes a conformational (3D) epitope, specifically binds to both the mutant and the starting SARS-CoV-2 Spike polypeptide fragments or their related fusion polypeptides. In the present invention “cross-reactivity” or a “cross-reacting antibody” is defined in terms of “binding affinity” which can be measured based on dissociation constant (K_{D}), off rate (k_{off}), and/or on rate (kₒₙ). Assays to test for the cross-reactivity are described herein or are known in the art. For example, binding assays may be performed in solution (e.g., Houghten, Bio/Techniques 13:412-421(1992)), on beads (e.g., Lam, Nature 354:82-84 (1991)), on chips (e.g., Fodor, Nature 364:555-556 (1993)), on bacteria (e.g., U.S. Pat. No. 5,223,409), on spores (e.g., U.S. Pat. Nos. 5,571,698, 5,403,484, 5,223,409), on plasmids (e.g., Cull et al., Proc. Natl. Acad. Sci. USA 89:1865-1869 (1992)) or on phage (e.g., Scott and Smith, Science 249:386-390 (1990); Devlin, Science 249:404-406 (1990); Cwirla et al., Proc. Natl. Acad. Sci. USA 87:6378-6382 (1990); and Felici, J. Mol. Biol. 222:301-310 (1991)).

In some embodiments, the SARS-CoV-2 Spike protein region from amino acids 300-600 can comprise one or more amino acid substitutions, such as at one or more sites selected from Ala 419, Ala 575, Val 576, Tyr 365, Ile 418, Leu 387, Leu 585, Ile 410, Tyr 423, Phe 497, and/or Leu 552 of SEQ ID NO:1, or the positions that align with those residues in any one of SEQ NOs: 2-100. In some embodiments, one or more of these sites may be substituted with a different amino acid residue.

In even further embodiments, mutations at the following sites may be made in the SARS-CoV-2 Spike protein, in comparison to the sequence of SEQ ID NO: 1 when constructing a SARS-CoV-2 Spike polypeptide fragment:
A. Trp 353, Tyr 365, Phe 392, Phe 400, Tyr 423, Phe 497, and/or Phe 543 of SEQ ID NO:1;
B. Val308, Ile326, Val350, Ile358, Ala363, Leu387, Val395, Ala397, Val401, Ile402, Ile410, Ile418, Ala419, Leu425, Val433, Ile434, Ala435, Leu492, Val510, Val5l, Val512, Leu513, Val524, Val539, Leu552, Ala575, Val576, and/or Leu585 of SEQ ID NO:1;
C. Ala 363, Ala 397, and/or Ala 575 of SEQ ID NO:1;
D. Cys 336 Ala/Cys 361 Ala, and/or Cys 379 Ala/Cys 432 Ala of SEQ ID NO:1; and/or
E. Ala 419, Ala 575, Val 576, Tyr 365, Ile 418, Leu 387, Leu 585, Ile 410, Tyr 423, Phe 497, and/or Leu 552 of SEQ ID NO:1.
   In some embodiments, the fragment comprises mutations at Y365, 1402, and/or V511 of SEQ ID NO:1. In some embodiments, the fragment comprises the following mutations Y365L, I402V, and/or V511A compared to the amino acid sequence of SEQ ID NO:1. Note that in considering the above positions and substitutions, the positions and substitutions are shown by reference to SEQ ID NO: 1. If the RBD domain of a different SARS-CoV-2 strain is used, the above positions of mutation are at the residues that align with the above positions from SEQ ID NO: 1, while the above L, V, and A substitutions occur at the residues that align with Y365, 1402, and V511 from SEQ ID NO: 1.

In some embodiments, the Spike polypeptide fragment comprises or consists of amino acids 316-594 of SEQ ID NO:1 (SEQ ID NO: 3), which is modified by mutations at the following sites: (A) Trp 353, Tyr 365, Phe 392, Phe 400, Tyr 423, Phe 497, and/or Phe 543 of SEQ ID NO:1; (B) Ile326, Val350, Ile358, Ala363, Leu387, Val395, Ala397, Val401, Ile402, Ile410, Ile418, Ala419, Leu425, Val433, Ile434, Ala435, Leu492, Val510, Val5l, Val512, Leu513, Val524, Val539, Leu552, Ala575, Val576, and/or Leu585 of SEQ ID NO:1; (C) Ala 363, Ala 397, and/or Ala 575 of SEQ ID NO:1; (D) Cys 336 Ala and Cys 361 Ala, and/or Cys 379 Ala and Cys 432 Ala; and/or (E) Ala 419, Ala 575, Val 576, Tyr 365, Ile 418, Leu 387, Leu 585, Ile 410, Tyr 423, Phe 497, and/or Leu 552 of SEQ ID NO:1. In some embodiments, the fragment comprises mutations at positions Y365, 1402 and/or V511 of SEQ ID NO:1. In some embodiments, the fragment comprises the following mutations Y365L, I402V, and/or V511A compared to the amino acid sequence of SEQ ID NO:1.

In some embodiments, the Spike polypeptide fragment comprises or consists of any one of SEQ ID Nos: 3-100, which is modified by mutations at the following sites, with residues provided with reference to SEQ ID NO: 1: (A) Trp 353, Tyr 365, Phe 392, Phe 400, Tyr 423, Phe 497, and/or Phe 543 of SEQ ID NO:1; (B) Ile326, Val350, Ile358, Ala363, Leu387, Val395, Ala397, Val401, Ile402, Ile410, Ile418, Ala419, Leu425, Val433, Ile434, Ala435, Leu492, Val510, Val511, Val512, Leu513, Val524, Val539, Leu552, Ala575, Val576, and/or Leu585 of SEQ ID NO:1; (C) Ala 363, Ala 397, and/or Ala 575 of SEQ ID NO:1; (D) Cys 336 Ala and Cys 361 Ala, and/or Cys 379 Ala and Cys 432 Ala; and/or (E) Ala 419, Ala 575, Val 576, Tyr 365, Ile 418, Leu 387, Leu 585, Ile 410, Tyr 423, Phe 497, and/or Leu 552 of SEQ ID NO:1. In some embodiments, the fragment comprises mutations at positions Y365, 1402 and/or V511 with reference to SEQ ID NO:1 (meaning the aligning positions in any one of SEQ ID NOs: 3-100). In some embodiments, the fragment comprises or consists of any one of SEQ ID NOs: 3-100, but with the following mutations: a leucine at position 365, a valine at position 402, and an alanine at position 511, the position numbering based on SEQ ID NO:1.

In yet other embodiments, the RBD domain may comprise an engineered glycosylation site. For example, in some embodiments the RBD domain may be engineered to comprise an N-X-T sequence at residues 519-521 as shown in SEQ ID NO: 1 (i.e., in the residues that align with residues 519-521 of SEQ ID NO: 1), wherein X is any residue other than proline. See, e.g., J. Shi et al., J. Virology, 96(17), 10.1128/jvi.00118-22 (2022). For example, such a mutation in SEQ ID NO: 3 would comprise an H519N and P521T substitution, at the residues corresponding to positions 519 and 521 of SEQ ID NO: 1. Similar substitutions may be made in other possible RBD domains, such as any one of SEQ ID NOs: 4-100, at positions that align with positions 519 and 521 of SEQ ID NO: 1 in order to engineer such an N-X-T glycosylation site in the RBD domain.

In addition to any of the above substitutions, in some embodiments a C-terminal tag is attached to the RBD domain, such as an EPEA (SEQ ID NO: 103) C-terminal tag, as shown, for example, in SEQ ID NOs: 4, 6, 78, 80, 82, 84, 86, 88, 90, 92, 94, 96, 98, and 100. Exemplary C-terminal tags are also further discussed below.

In some embodiments, a composition may comprise a mixture of SARS-CoV-2 Spike polypeptide fragments or fusion polypeptides derived therefrom, comprising one or more different mutant SARS-CoV-2 Spike polypeptide fragments as described herein and optionally further comprising the starting SARS-CoV-2 Spike polypeptide fragment from which the one or more different mutants are derived. For example, in some embodiments, such a mixture may comprise Spike polypeptide fragments with mutations at one or more of Y365, 1402, and V511 with reference to SEQ ID NO: 1 (e.g. Y365L, I402V, and/or V511 Å), optionally along with the unmutated starting polypeptide fragment, such as a mixture of one, two, or three different Spike polypeptides, each with a mutation at one of those three locations, with the mixture optionally further comprising the original unmutated starting Spike polypeptide fragment. In some cases, providing such a mixture may result in a higher RBD-specific B-cell response compared to a composition comprising only the unmutated starting Spike polypeptide fragment.

### Polynucleotides and Host Cells and Production Methods

In some embodiments, a polynucleotide encoding the above fusion polypeptides can be incorporated into a vector for expression in a host cell, or in other embodiments may be synthesized in vitro.

Methods known to those skilled in the art can be used to construct expression vectors containing the coding sequences for the SARS-CoV-2 Spike polypeptide fragments or fusion polypeptides herein, for expression under the control of appropriate promoters or enhancers or other gene expression signals. These methods include, for example, in vitro recombinant DNA techniques, synthetic techniques, and in vivo genetic recombination. The disclosure, thus, further provides polynucleotides encoding the fusion polypeptides and SARS-CoV-2 Spike polypeptide fragments, optionally included within appropriate expression vectors for expression in a host cell.

The expression vector(s) can be transferred to a host cell by conventional techniques and the transfected cells then cultured by conventional techniques to produce the encoded polypeptide. coding sequences, express the fusion polypeptides and SARS-CoV-2 Spike polypeptide fragments or to induce production of antibodies against the fusion polypeptides and SARS-CoV-2 Spike polypeptide fragments. Host cells include, for example, yeast cells, insect cells, and mammalian cells (e.g., COS, CHO, BHK, 293, 3T3 cells). For example, mammalian cells such as Chinese hamster ovary cells (CHO), in conjunction with a vector such as the major intermediate early gene promoter element from human cytomegalovirus is an effective expression system (Foecking et al., Gene 45:101 (1986); Cockett et al., Bio/Technology 8:2 (1990)).

Appropriate cell lines or host systems can be chosen to ensure the correct modification and processing of the foreign protein expressed, to this end, eukaryotic host cells which possess the cellular machinery for proper processing of the primary transcript, glycosylation, and phosphorylation of the gene product may be used. Such mammalian host cells include, but are not limited to, CHO, VERY, BHK, Hela, COS, NSO, MDCK, 293, 3T3, W138, breast cancer cell lines such as, for example, BT483, Hs578T, HTB2, BT20 and T47D, and normal mammary gland cell lines such as, for example, CRL7030 and HsS78Bst.

Embodiments of the disclosure also include methods of producing the SARS-CoV-2 Spike polypeptide fragments or fusion polypeptides herein in a host cell, such as by incubating host cells comprising polynucleotides or vectors encoding the SARS-CoV-2 Spike polypeptide fragments or fusion polypeptides under conditions allowing for expression of the SARS-CoV-2 Spike polypeptide fragments or fusion polypeptides, and optionally further isolating the SARS-CoV-2 Spike polypeptide fragments or fusion polypeptides from the host cells. In some cases, further purification of the SARS-CoV-2 Spike polypeptide fragments or fusion polypeptides may be performed, such as to obtain a highly purified composition comprising the SARS-CoV-2 Spike polypeptide fragments or fusion polypeptides for administration to a human or other animal.

### Pharmaceutical Compositions

The SARS-CoV-2 Spike polypeptide fragments or fusion polypeptides may provided in an isolated form, and may also be substantially purified, as noted above. In some cases, a mixture of different fusion polypeptides, for example, with more than one different SARS CoV-2 Spike polypeptide fragment sequence, may be prepared and administered to a human or other animal. In other cases, only one SARS-CoV-2 Spike polypeptide fragment or fusion polypeptide, i.e., with one single amino acid sequence, is administered. In some cases, a polynucleotide encoding the fusion polypeptide or SARS-CoV-2 Spike polypeptide fragment or a host cell comprising such a polynucleotide is administered to the human or animal such that the fusion polypeptide or SARS-CoV-2 Spike polypeptide fragment is produced from the polynucleotide or host cell after administration.

A pharmaceutical composition may comprise a SARS-CoV-2 Spike polypeptide fragment or fusion polypeptide as described herein, or a polynucleotide encoding the polypeptide, or a host cell expressing the polynucleotide, along with one or more pharmaceutically acceptable carriers, adjuvants, excipients, diluents, fillers, buffers, stabilizers, preservatives, lubricants, or other materials well known to those skilled in the art. Suitable materials will be sterile and pyrogen-free, with a suitable isotonicity and stability. Examples include sterile saline (e.g., 0.9% NaCl), water, dextrose, glycerol, ethanol or the like or combinations thereof. Such materials should be non-toxic and should not interfere with the efficacy of the active compound. The precise nature of the carrier or other material will depend on the route of administration, which may be by bolus, infusion, injection or any other suitable route, such as, including but not limited to, oral (e.g., by ingestion); and parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly. In some embodiments, administration will be by injection. In other cases, administration may be intranasal or otherwise by inhalation methods.

The composition may further contain auxiliary substances such as wetting agents, emulsifying agents, pH buffering agents or the like. Suitable carriers, excipients, etc. can be found in standard pharmaceutical texts, for example, Remington's Pharmaceutical Sciences, 18th edition, Mack Publishing Company, Easton, Pa., 1990.

In some embodiments, a pharmaceutical composition comprises a SARS-CoV-2 Spike polypeptide fragment or fusion polypeptide in a liquid formulation, while in other cases, the composition comprises a SARS-CoV-2 Spike polypeptide fragment or fusion polypeptide in lyophilized form. A pharmaceutical composition in some cases may conveniently be presented in unit dosage form, or alternatively, may be presented so as to be dispensed in an appropriate dosage. Compositions may be prepared in the form of a concentrate for subsequent dilution, or may be in the form of divided doses ready for administration. Alternatively, the reagents may be provided separately within a kit, for mixing prior to administration to a human or animal subject.

In some cases, a pharmaceutical composition, such as in the form of a kit comprising more than one agent, may also include one or more adjuvants. Exemplary adjuvants that may be used with the SARS-CoV-2 Spike polypeptide fragment or fusion polypeptide herein include an aluminum salt, such as aluminum hydroxide, such as Alhydrogel®, as well as Toll-like receptor agonists (TLR agonists). Exemplary TLR agonists include agonists of TLR3, TLR4, TLR7, TLR8, TLR7/8, or TLR9. In some cases, TLR agonists are CpG dinucleotide agonists, such as CpG1018 or others approved for human use. In some cases, a TLR9 agonist is included as an adjuvant. In some cases, both a TLR9 agonist and an aluminum salt are provided as agonists, such as Alhydrogel® and a CpG dinucleotide agonist of TLR9 such as CpG1018 or the like. In other cases, only an aluminum salt is provided as an agonist, such as aluminum hydroxide. In some cases, an aluminum salt is provided, such as aluminum hydroxide, as an agonist, while a CpG dinucleotide is not provided. In some cases, an aluminum salt is provided, such as aluminum hydroxide, as an agonist, while a TLR9 agonist is not provided. In some cases, an aluminum salt is provided, such as aluminum hydroxide, as an agonist, while a TLR agonist is not provided.

### Therapeutic Uses of the SARS-CoV-2 Polypeptide Fragments and Fusion Polypeptides

The present disclosure also encompasses methods of vaccinating an animal, such as a human, comprising administering the fusion polypeptide or SARS-CoV-2 Spike polypeptide fragment to the animal. The administered fusion polypeptide or SARS-CoV-2 Spike polypeptide fragment may be as described in the sections above. For example, in some embodiments, a fusion polypeptide or a SARS-CoV-2 Spike polypeptide fragment that comprises at least a portion of the N-terminal domain (NT), domains CD1, RBM, and CD2, and at least a portion of the C-terminal domain 1 (CTD1) is administered. In the case of a fusion polypeptide, the fusion polypeptide may comprise a heterologous N- or C-terminal tag comprising at least two, at least three, at least four, at least five, or at least six, or 2, 3, 4, 5, or 6 amino acids that fused to the N- or C-terminus of the Spike polypeptide fragment.

In some embodiments, the N- and C-terminal residues of the Spike polypeptide fragment, e.g., drawn from the portions of the N-terminal domain and CTD1 included in the fragment, are comprised within an antiparallel beta-sheet. For example, in some embodiments, a Spike polypeptide fragment or fusion polypeptide encompasses residues 311-600 of SEQ ID NO: 1 (i.e., SEQ ID NO: 7). In some cases, such a Spike polypeptide fragment could begin at a residue from 311 to 316 of SEQ ID NO: 1 such as at 311, 312, 313, 314, 315, or 316 (or at equivalent positions in a SARS-CoV-2 polypeptide that aligns with those residues). In some cases, such a Spike polypeptide fragment could end at a residue from 594 to 600 of SEQ ID NO: 1 such as at 594, 595, 596, 597, 598, 599, or 600 (or at equivalent positions in a SARS-CoV-2 polypeptide that aligns with those residues). In some cases, such a Spike polypeptide fragment could begin at a residue from 311 to 316 of SEQ ID NO: 1 such as at 311, 312, 313, 314, 315, or 316 (or at equivalent positions in a SARS-CoV-2 polypeptide that aligns with those residues) and could end at a residue from 594 to 600 of SEQ ID NO: 1 such as at 594, 595, 596, 597, 598, 599, or 600 (or at equivalent positions in a SARS-CoV-2 polypeptide that aligns with those residues).

In some embodiments, the SARS-CoV-2 Spike protein fragment or fusion polypeptide comprises residues 316-594 of a SARS-CoV-2 Spike protein as shown in SEQ ID NO: 1, which also corresponds to SEQ ID NO: 3. Positions 316-594 of SEQ ID NO: 1 (SEQ ID NO: 3) include a portion of the N-terminal domain, domains CD1, RBM, and CD2, and a portion of CTD1. In other embodiments, the SARS-CoV-2 Spike protein fragment or fusion polypeptide comprises an equivalent portion of a SARS-CoV-2 Spike protein of a different amino acid sequence that aligns with residues 316-594 of SEQ ID NO: 1, as noted above. Examples are SEQ ID NO: 5 as well as any of SEQ ID NO: 8 (corresponding to residues 303-580 of SEQ ID NO: 2), and SEQ ID Nos: 9-76 shown in the Table 1 above, and SEQ ID NOs: 77, 79, 81, 83, 85, 87, 89, 91, 93, 95, 97, and 99, and the sequences shown in FIG. 11.

In some embodiments, the SARS-CoV-2 Spike polypeptide fragment or fusion polypeptide comprises or consists of amino acids 316-594 of SEQ ID NO:1 (SEQ ID NO: 3), SEQ ID NO: 5, amino acids 303-580 of SEQ ID NO:2 (SEQ ID NO: 8), or equivalent fragments in other Spike glycoproteins of Coronaviruses, such as those as shown in the above table of sequences (Table 1; SEQ ID NO: 9-76 as well as 77, 79, 81, 83, 85, 87, 89, 91, 93, 95, 97, and 99, and FIG. 11). In some embodiments, the Spike polypeptide fragment or fusion polypeptide comprises or consists of an amino acid sequence that aligns with that of residues 316-594 of SEQ ID NO: 1 and/or residues 303-580 of SEQ ID NO: 2 and/or the amino acid sequence of any one of SEQ ID Nos: 3, 5, 7, or 8 and/or any one of SEQ ID NOs: 77, 79, 81, 83, 85, 87, 89, 91, 93, 95, 97, or 99 using an appropriate sequence alignment platform.

In some embodiments, in the fusion polypeptide, an N- or C-tag is placed at the N- and/or C-terminus of the Spike polypeptide fragment. The tag may comprise at least two, at least three, at least four, at least five, at least six, or 2, 3, 4, 5, or 6 amino acid residues. In some embodiments, the tag comprises the amino acid sequence EPEA (SEQ ID NO: 103). In some embodiments, the tag may contribute to protein purification by being recognized by particular antibodies, nanobodies, or other protein capture reagents against the amino acid sequence epitope of the tag, for example. In some embodiments, one or more residues of the tag may further be comprised within the above-described anti-parallel beta sheet involving the N- and C-termini of the Spike polypeptide fragment. In other embodiments, one or more or all of the residues of the tag may be unstructured, i.e., not involved in contacts with residues of the Spike polypeptide fragment.

In some cases, the fusion polypeptide herein comprises a SARS-CoV-2 Spike polypeptide fragment comprising amino acids 316-594 of SEQ ID NO: 1 (SEQ ID NO: 3). In some cases, the fusion polypeptide herein comprises a SARS-CoV-2 Spike polypeptide fragment consisting of amino acids 316-594 of SEQ ID NO: 1 (SEQ ID NO: 3). In some cases, the fusion polypeptide herein comprises a SARS-CoV-2 Spike polypeptide fragment comprising the amino acid sequence of SEQ ID NO: 5. In some cases, the fusion polypeptide herein comprises a SARS-CoV-2 Spike polypeptide fragment consisting of the amino acid sequence of SEQ ID NO: 5. In some cases, the fusion polypeptide herein comprises a SARS-CoV-2 Spike polypeptide fragment comprising an amino acid sequence at least 90%, at least 95%, at least 97%, at least 98%, or at least 99% identical to the amino acid sequence of SEQ ID NO: 3 or SEQ ID NO: 5. In any of those cases, in some embodiments the fusion polypeptide comprises a C-terminal tag, such as a C-terminal tag comprising or consisting of the amino acid sequence of EPEA (SEQ ID NO: 103). Thus, in some embodiments, the fusion polypeptide comprises or consists of the amino acid sequence of SEQ ID NO: 4 or SEQ ID NO: 6, or of any one of SEQ ID Nos: 3, 5, or 8-76 followed by the amino acid sequence EPEA (SEQ ID NO: 103), or any one of SEQ ID NOs: 77, 79, 81, 83, 85, 87, 89, 91, 93, 95, 97, or 99 optionally followed by the amino acid sequence EPEA (SEQ ID NO: 103), or any one of SEQ ID NOs: 78, 80, 82, 84, 86, 88, 90, 92, 94, 96, 98, or 100.

In some cases, the fusion polypeptide herein comprises a SARS-CoV-2 Spike polypeptide fragment comprising the amino acid sequence of SEQ ID NO: 77. In some cases, the fusion polypeptide herein comprises a SARS-CoV-2 Spike polypeptide fragment consisting of the amino acid sequence of SEQ ID NO: 77. In some cases, the fusion polypeptide herein comprises a SARS-CoV-2 Spike polypeptide fragment comprising an amino acid sequence at least 90%, at least 95%, at least 97%, at least 98%, or at least 99% identical to the amino acid sequence of SEQ ID NO: 77. In some such cases, the sequence is followed by the amino acid sequence EPEA (SEQ ID NO: 103).

In some cases, the fusion polypeptide herein comprises a SARS-CoV-2 Spike polypeptide fragment comprising the amino acid sequence of SEQ ID NO: 79. In some cases, the fusion polypeptide herein comprises a SARS-CoV-2 Spike polypeptide fragment consisting of the amino acid sequence of SEQ ID NO: 79. In some cases, the fusion polypeptide herein comprises a SARS-CoV-2 Spike polypeptide fragment comprising an amino acid sequence at least 90%, at least 95%, at least 97%, at least 98%, or at least 99% identical to the amino acid sequence of SEQ ID NO: 79. In some such cases, the sequence is followed by the amino acid sequence EPEA (SEQ ID NO: 103).

In some cases, the fusion polypeptide herein comprises a SARS-CoV-2 Spike polypeptide fragment comprising the amino acid sequence of SEQ ID NO: 81. In some cases, the fusion polypeptide herein comprises a SARS-CoV-2 Spike polypeptide fragment consisting of the amino acid sequence of SEQ ID NO: 81. In some cases, the fusion polypeptide herein comprises a SARS-CoV-2 Spike polypeptide fragment comprising an amino acid sequence at least 90%, at least 95%, at least 97%, at least 98%, or at least 99% identical to the amino acid sequence of SEQ ID NO: 81. In some such cases, the sequence is followed by the amino acid sequence EPEA (SEQ ID NO: 103).

In some cases, the fusion polypeptide herein comprises a SARS-CoV-2 Spike polypeptide fragment comprising the amino acid sequence of SEQ ID NO: 83. In some cases, the fusion polypeptide herein comprises a SARS-CoV-2 Spike polypeptide fragment consisting of the amino acid sequence of SEQ ID NO: 83. In some cases, the fusion polypeptide herein comprises a SARS-CoV-2 Spike polypeptide fragment comprising an amino acid sequence at least 90%, at least 95%, at least 97%, at least 98%, or at least 99% identical to the amino acid sequence of SEQ ID NO: 83. In some such cases, the sequence is followed by the amino acid sequence EPEA (SEQ ID NO: 103).

In some cases, the fusion polypeptide herein comprises a SARS-CoV-2 Spike polypeptide fragment comprising the amino acid sequence of SEQ ID NO: 85. In some cases, the fusion polypeptide herein comprises a SARS-CoV-2 Spike polypeptide fragment consisting of the amino acid sequence of SEQ ID NO: 85. In some cases, the fusion polypeptide herein comprises a SARS-CoV-2 Spike polypeptide fragment comprising an amino acid sequence at least 90%, at least 95%, at least 97%, at least 98%, or at least 99% identical to the amino acid sequence of SEQ ID NO: 85. In some such cases, the sequence is followed by the amino acid sequence EPEA (SEQ ID NO: 103).

In some cases, the fusion polypeptide herein comprises a SARS-CoV-2 Spike polypeptide fragment comprising the amino acid sequence of SEQ ID NO: 87. In some cases, the fusion polypeptide herein comprises a SARS-CoV-2 Spike polypeptide fragment consisting of the amino acid sequence of SEQ ID NO: 87. In some cases, the fusion polypeptide herein comprises a SARS-CoV-2 Spike polypeptide fragment comprising an amino acid sequence at least 90%, at least 95%, at least 97%, at least 98%, or at least 99% identical to the amino acid sequence of SEQ ID NO: 87. In some such cases, the sequence is followed by the amino acid sequence EPEA (SEQ ID NO: 103).

In some cases, the fusion polypeptide herein comprises a SARS-CoV-2 Spike polypeptide fragment comprising the amino acid sequence of SEQ ID NO: 89. In some cases, the fusion polypeptide herein comprises a SARS-CoV-2 Spike polypeptide fragment consisting of the amino acid sequence of SEQ ID NO: 89. In some cases, the fusion polypeptide herein comprises a SARS-CoV-2 Spike polypeptide fragment comprising an amino acid sequence at least 90%, at least 95%, at least 97%, at least 98%, or at least 99% identical to the amino acid sequence of SEQ ID NO: 89. In some such cases, the sequence is followed by the amino acid sequence EPEA (SEQ ID NO: 103).

In some cases, the fusion polypeptide herein comprises a SARS-CoV-2 Spike polypeptide fragment comprising the amino acid sequence of SEQ ID NO: 91. In some cases, the fusion polypeptide herein comprises a SARS-CoV-2 Spike polypeptide fragment consisting of the amino acid sequence of SEQ ID NO: 91. In some cases, the fusion polypeptide herein comprises a SARS-CoV-2 Spike polypeptide fragment comprising an amino acid sequence at least 90%, at least 95%, at least 97%, at least 98%, or at least 99% identical to the amino acid sequence of SEQ ID NO: 91. In some such cases, the sequence is followed by the amino acid sequence EPEA (SEQ ID NO: 103).

In some cases, the fusion polypeptide herein comprises a SARS-CoV-2 Spike polypeptide fragment comprising the amino acid sequence of SEQ ID NO: 93. In some cases, the fusion polypeptide herein comprises a SARS-CoV-2 Spike polypeptide fragment consisting of the amino acid sequence of SEQ ID NO: 93. In some cases, the fusion polypeptide herein comprises a SARS-CoV-2 Spike polypeptide fragment comprising an amino acid sequence at least 90%, at least 95%, at least 97%, at least 98%, or at least 99% identical to the amino acid sequence of SEQ ID NO: 93. In some such cases, the sequence is followed by the amino acid sequence EPEA (SEQ ID NO: 103).

In some cases, the fusion polypeptide herein comprises a SARS-CoV-2 Spike polypeptide fragment comprising the amino acid sequence of SEQ ID NO: 95. In some cases, the fusion polypeptide herein comprises a SARS-CoV-2 Spike polypeptide fragment consisting of the amino acid sequence of SEQ ID NO: 95. In some cases, the fusion polypeptide herein comprises a SARS-CoV-2 Spike polypeptide fragment comprising an amino acid sequence at least 90%, at least 95%, at least 97%, at least 98%, or at least 99% identical to the amino acid sequence of SEQ ID NO: 95. In some such cases, the sequence is followed by the amino acid sequence EPEA (SEQ ID NO: 103).

In some cases, the fusion polypeptide herein comprises a SARS-CoV-2 Spike polypeptide fragment comprising the amino acid sequence of SEQ ID NO: 97. In some cases, the fusion polypeptide herein comprises a SARS-CoV-2 Spike polypeptide fragment consisting of the amino acid sequence of SEQ ID NO: 97. In some cases, the fusion polypeptide herein comprises a SARS-CoV-2 Spike polypeptide fragment comprising an amino acid sequence at least 90%, at least 95%, at least 97%, at least 98%, or at least 99% identical to the amino acid sequence of SEQ ID NO: 97. In some such cases, the sequence is followed by the amino acid sequence EPEA (SEQ ID NO: 103).

In some cases, the fusion polypeptide herein comprises a SARS-CoV-2 Spike polypeptide fragment comprising the amino acid sequence of SEQ ID NO: 99. In some cases, the fusion polypeptide herein comprises a SARS-CoV-2 Spike polypeptide fragment consisting of the amino acid sequence of SEQ ID NO: 99. In some cases, the fusion polypeptide herein comprises a SARS-CoV-2 Spike polypeptide fragment comprising an amino acid sequence at least 90%, at least 95%, at least 97%, at least 98%, or at least 99% identical to the amino acid sequence of SEQ ID NO: 99. In some such cases, the sequence is followed by the amino acid sequence EPEA (SEQ ID NO: 103).

A vaccination strategy can be based on one or more administrations of the SARS-CoV-2 Spike polypeptide fragment or fusion polypeptide described herein, for example, as necessary to enable the development of memory B cells, memory T cells, and long-lived plasma cells (LLPCs) against the polypeptide fragment or fusion polypeptide. Vaccination can be conducted either prophylactically or therapeutically. The SARS-CoV-2 Spike polypeptide fragment or fusion polypeptide can be derived from either the same SAR_S-CoV-2 starting protein or from multiple SARS-CoV-2 starting proteins. While prophylactic vaccination strategies aim to stimulate the subject's immune system in developing preventive adaptive immunity to a pathogen, a therapeutic vaccination strategy conducted after the disease has been already established may be used to improve a clinical situation present in the subject.

In some cases, the SARS-CoV-2 Spike polypeptide fragment or fusion polypeptide may be proteolytically processed in the body. For example, antigens may be processed in Antigen Presenting Cells (APCs) after endocytosis and fusion of an endosome with a lysosome. The endosome then merges with an exocytic vesicle from the Golgi apparatus containing class II MHC molecules, to which the resultant peptides bind. The MHC-peptide complex then trafficks to the plasma membrane where the antigen is available for display to CD4⁺ T cells.

A SARS-CoV-2 Spike polypeptide fragment or fusion polypeptide for use as a vaccine may be administered by a variety of methods or may be included as part of a pharmaceutical composition as described in the sections above. For example, a vaccine composition may be administered by injection or inhalation in some embodiments. The SARS-CoV-2 Spike polypeptide fragment or fusion polypeptide may also be administered in nucleic acid form, e.g., in a DNA or RNA vector or in naked DNA or RNA form, such as in a delivery vehicle such as a liposome or similar particle, or via a transformed cell, so that the polypeptide is produced from the encoding nucleic acids in the body.

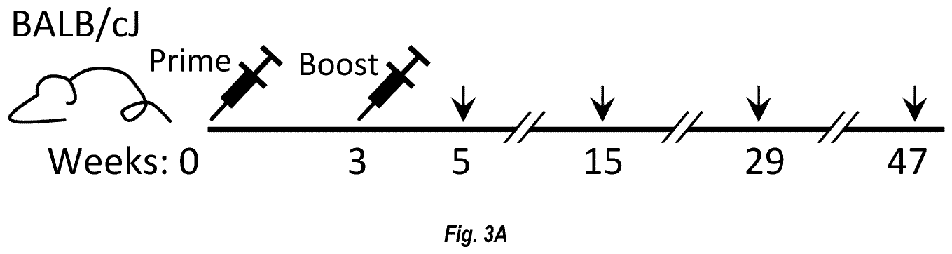

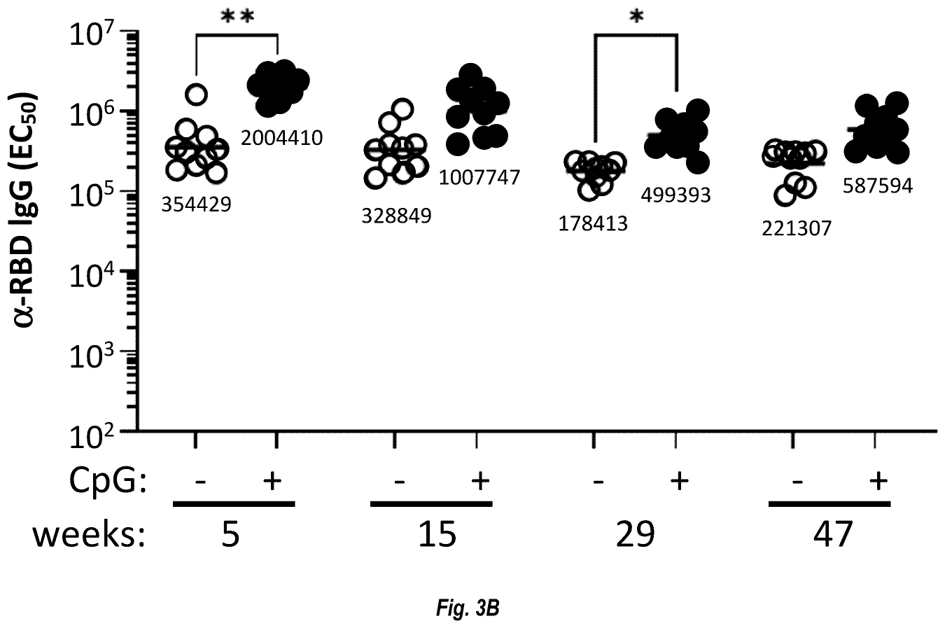

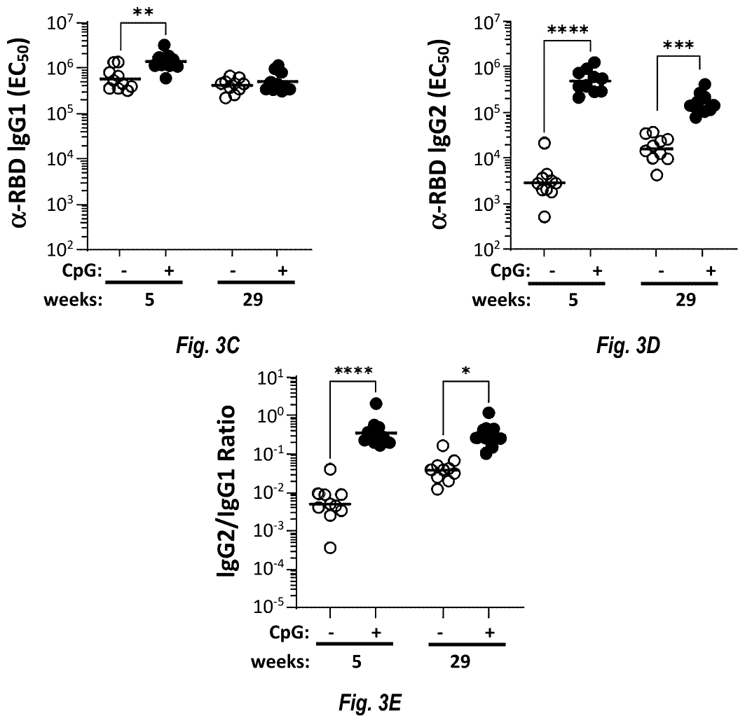

In some embodiments, the SARS-CoV-2 Spike polypeptide fragment or fusion polypeptide may be administered in a single dose, for instance, every 6 months, every 9 months, every 12 months (i.e., annually or every year), or every 1.5 years, or every 2 years. The use of certain adjuvants and other strategies, for instance to achieve potent CD4+ and CD8⁺ T cell responses, may also be employed to achieve strong and protective immune responses in elderly patients (Weinberger, Immunity & Ageing 15:3 (2018)). In some cases, the SARS-CoV-2 Spike polypeptide fragment or fusion polypeptide may be administered together with at least one adjuvant, either concurrently (meaning in the same setting, such as, at one point in time or in the context of a single visit to a doctor or medical clinic), or sequentially (meaning at different points in time, such as on different days or after a period of 7-60 days, such as 7-10, 15-45, 15-30, 7-15, 7-30, or 45-60 days. Exemplary adjuvants that may be used with the SARS-CoV-2 Spike polypeptide fragment or fusion polypeptide herein include an aluminum salt, such as aluminum hydroxide, such as Alhydrogel®, as well as Toll-like receptor agonists (TLR agonists). Exemplary TLR agonists include agonists of TLR3, TLR4, TLR7, TLR8, TLR7/8, or TLR9. In some cases, TLR agonists are CpG di-nucleotide agonists, such as CpG1018 or others approved for human use. In some cases a TLR9 agonist is included as an adjuvant. In some cases, both a TLR9 agonist and an aluminum salt are provided as agonists, such as Alhydrogel® and a CpG di-nucleotide agonist of TLR9 such as CpG1018 or the like. In some embodiments, the adjuvant includes an aluminum salt such as aluminum hydroxide, and does not comprise a TLR agonist. In some embodiments, the adjuvant includes an aluminum salt such as aluminum hydroxide, and does not comprise a TLR9 agonist. In some embodiments, the adjuvant includes an aluminum salt such as aluminum hydroxide, and does not comprise a CpG di-nucleotide agonist. For example, experiments described below indicate that a SARS-CoV-2 Spike fragment-comprising fusion polypeptide surprisingly showed a long durability of response, such as up to 12 months, with an aluminum salt adjuvant alone as well as with both the aluminum salt and a CpG di-nucleotide adjuvant. (See, e.g., FIGS. 2B and 3B.)

In some embodiments, vaccination with the SARS-CoV-2 Spike polypeptide fragment or fusion polypeptide herein provides a sufficiently durable immune response, such as an antibody-mediated immune response, in subjects that the SARS-CoV-2 Spike polypeptide fragment or fusion polypeptide may be administered every 6 months, every 9 months, every 12 months (i.e., annually or every year), or every 1.5 years, or every 2 years. In some cases, the SARS-CoV-2 Spike polypeptide fragment or fusion polypeptide may be administered at least annually, in comparison to recommended administration of current SARS-CoV-2 vaccines about every 4-6 months. For example, in some embodiments, the antibody-mediated immune response from a subject administered the pharmaceutical composition, such as a murine subject or a human subject, does not wane after at least 6 months, at least 9 months, or at least 12 months, thus allowing for less frequent administrations. For example, in some embodiments, the anti-RBD IgG antibody titer in a blood sample from a subject administered the pharmaceutical composition, such as a murine subject or a human subject, is not significantly reduced or remains at a relatively high level indicating that the immune response has not waned after at least 6 months, at least 9 months, or at least 12 months, thus allowing for less frequent administrations.

### Uses of the SARS-CoV-2 Polypeptide Fragments and Fusion Polypeptides to Generate Antibodies

The SARS-CoV-2 Spike polypeptide fragments and fusion polypeptides herein can also be used to generate antibodies by methods well known by the skilled artisan, such as, for example, methods described in the art. See, for instance, Sutcliffe et al., supra; Wilson et al., supra; Chow et al., Proc. Natl. Acad. Sci. USA 82:910-914 (1985); and Bittle et al., J. Gen. Virol. 66:2347-2354 (1985). If in vivo immunization is used, animals may be immunized with a SARS-CoV-2 Spike polypeptide fragment or fusion polypeptide described herein. Animals such as rabbits, rats, mice, llamas, camels, and/or cows can be immunized with the SARS-CoV-2 Spike polypeptide fragment or fusion polypeptide. For instance, intraperitoneal and/or intradermal injection of emulsions containing a SARS-CoV-2 Spike polypeptide fragment or fusion polypeptide, optionally together with a carrier protein and Freund's adjuvant or any other adjuvant known for stimulating an immune response may be used. Several booster injections may be needed, for instance, at intervals of about two weeks, to provide a useful titer of anti-SARS-CoV-2 Spike polypeptide antibody which can be detected, for example, by ELISA assay using free SARS-CoV-2 Spike fragments adsorbed, directly or indirectly (e.g., via a biotinylated AviTag™), to a solid surface. The titer of anti-SARS-CoV-2 Spike antibodies in serum from an immunized animal may be increased by selection of anti-SARS-CoV-2 Spike antibodies, for instance, by adsorption to a SARS-CoV-2 Spike fragment on a solid support and elution of the selected antibodies according to methods well known in the art. Such selections could also be done using the SARS-CoV-2 starting protein. In some instances, one may wait at least 3 months, at least 4 months, at least 6 months, at least 9 months, or at least 12 months to collect antibodies from serum.

Additionally, antibodies generated by the disclosed methods can be affinity matured using display technology, such as for example, phage display, yeast display or ribosome display. In one example, single chain antibody molecules (“scFvs”) displayed on the surface of phage particles are screened to identify those scFvs that immunospecifically bind to SARS-CoV-2 Spike polypeptide fragments or full-length Spike. The present invention encompasses both scFvs, Fabs, and portions thereof that are identified to immunospecifically bind to the SARS-CoV-2 polypeptide fragment or fusion polypeptide herein, and/or the SARS-CoV-2 starting protein. Such scFvs and Fabs can routinely be “converted” to immunoglobulin molecules by inserting, for example, the nucleotide sequences encoding the VH and/or VL domains of the scFv or Fab into an expression vector containing the constant domain sequences and engineered to direct the expression of the immunoglobulin molecule.

Various further aspects and embodiments of the present invention will be apparent to those skilled in the art in view of the present disclosure.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such these are within the scope of the present invention. All documents and sequence database entries mentioned in this specification are incorporated herein by reference in their entirety for all purposes. The invention is further described below, with reference to the following examples.

### EXAMPLES

As described in the following Examples, a novel protein component vaccine was designed based on the RBD and RBD-adjacent sequences of the SARS-CoV-2 spike protein. By focusing the immune response on the region of the spike protein where the bulk of the epitopes for neutralizing (including broadly neutralizing) antibodies reside (34, 35), a high potency vaccine could be enabled. A recombinant immunogen that would be stable, highly soluble, capable of expression at high levels, and amenable to streamlined purification protocols was also designed. A recombinant immunogen with these features could endow the vaccine with relatively uncomplicated manufacturing and distribution requirements that would facilitate its adoption on a global scale.

The Examples that follow relate to a 2-dose prime-boost regimen in BALB/cJ mice and Syrian golden hamsters with the resultant vaccine, MT-001. The results show that the vaccinated mice exhibited peak anti-spike IgG ELISA titers comparable to those reported in studies with mRNA vaccines from Pfizer/BioNTech (BNT162b2) and Moderna (mRNA-1273) at similar doses in the same animal model (36, 37). When adjuvanted with both aluminum hydroxide (Alhydrogel®) and the TLR-9 CpG agonist ODN1826, the MT-001 vaccine in BALB/cJ mice showed a balanced Th1/Th2 response as well as peak anti-spike RBD IgG mid-point ELISA titers on the order of 10⁶ GMT. Syrian golden hamsters vaccinated with MT-001 adjuvanted with alum plus CpG exhibited undetectable levels of SARS-CoV-2 in lung tissue four days after intranasal challenge with SARS-CoV-2/US-WA1. Also, anti-spike IgG ELISA titers in sera from vaccinated mice were found to be durable, i.e., with EC50 in the range of 10⁵-10⁶ up to 12 months post-vaccination. Furthermore, results showed an increased breadth of the response, with significant neutralization titers against the Omicron BA.1 variant at this timepoint.

Combined, these attributes make MT-001 a compelling candidate for a next-generation COVID-19 vaccine. MT-001 could be particularly valuable as an annual booster to routinely augment immunity in individuals with diverse histories of vaccination, SARS-CoV-2 infection, and/or predispositions resulting in an immunocompromised state.

### Example 1: Methods

### 1. Design and Expression of MT-001

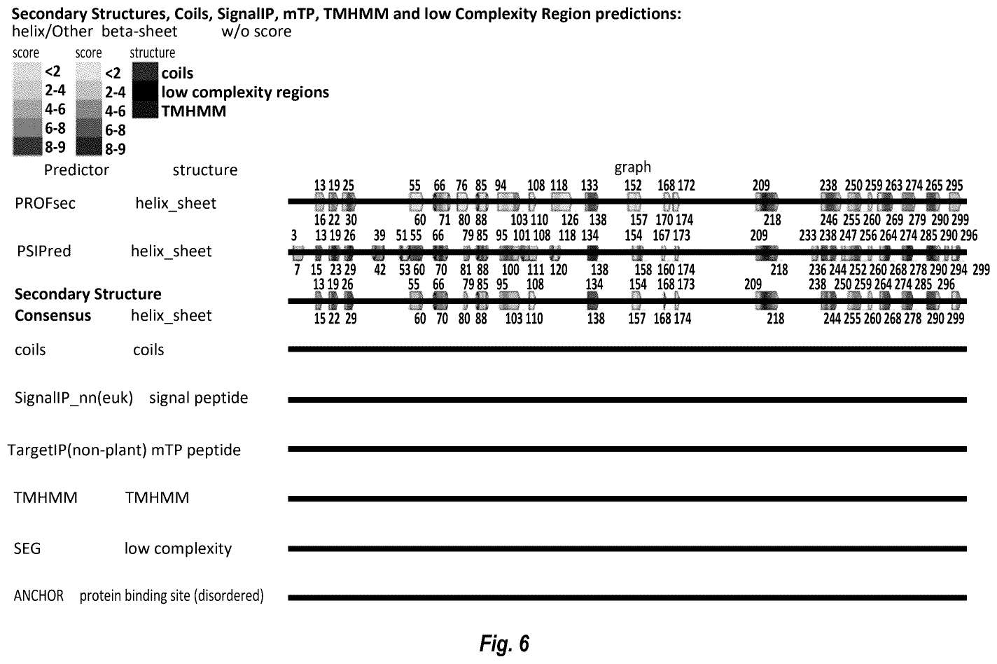

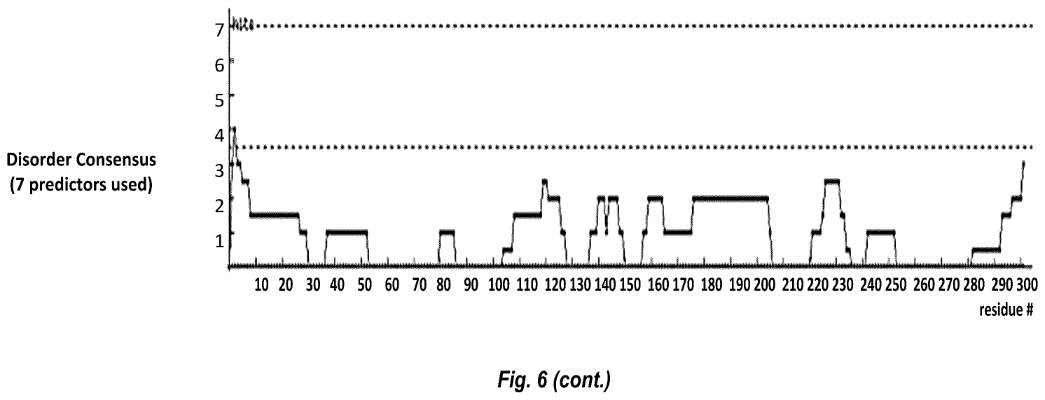

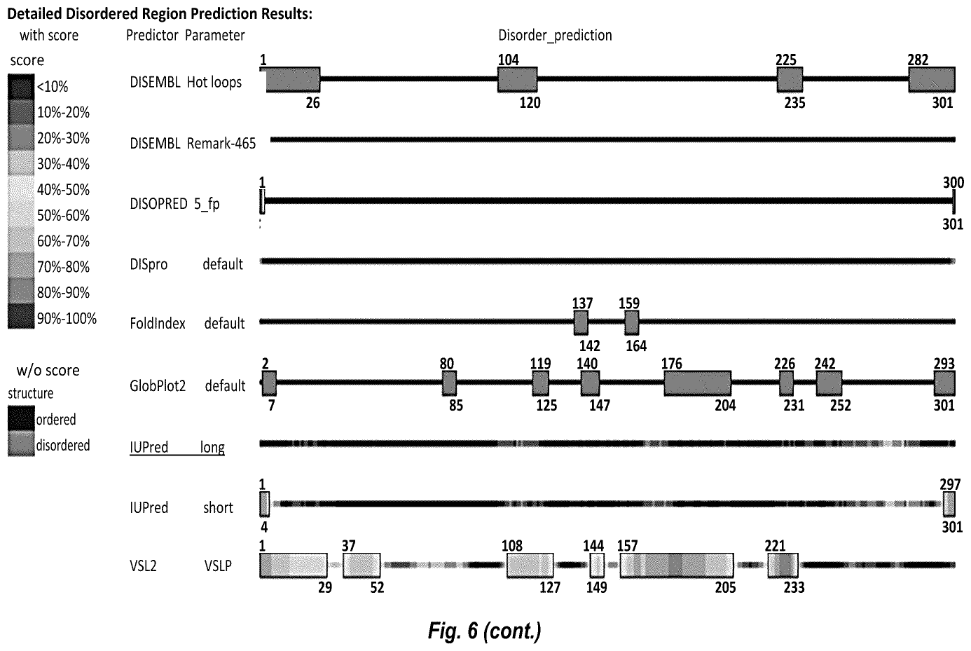

The sequence of the ancestral SARS-CoV-2 Wu-1 strain spike protein (YP_009724390.1) was analyzed using publicly-available bioinformatics tools for calculating structural, biophysical, and biochemical properties of potential constructs. Access to such tools can be found on the DisMeta server (montelionelab.chem.rpi.edu/dismeta/); shown in FIG. 6 is an example output from DisMeta for residues 300-600 of the spike protein. The results of these analyses were used to parse the sequence to yield a final expression construct designed to encompass the annotated receptor binding domain (residues 319-541), but with the construct N- and C-termini extended with the aim of including additional spike protein structural elements flanking the RBD domain that might promote proper domain folding and improved stability. The resulting RBD construct, MT-001, corresponded to residues 316-594 of spike fused to a C-terminal C-tag. The MT-001 construct was codon-optimized and expressed via a secretion vector in HEK293 cells by ATUM, Inc., Newark, CA, and purified in a single affinity chromatography step using the CaptureSelect™ C-tagXL system (ThermoFisher) (38, 39). The final purified yield was >160 mg from 1 L suspension culture. The purified protein was >96% monomeric with an apparent molecular weight of 39.4 kDa (calculated 31.6 kDa) by HPLC-SEC and had an apparent purity of >99% by capillary electrophoresis (FIGS. 7A-B). Solubility was determined to be >10 mg/ml in PBS (137 mM NaCl, 2.7 mM KCl, 8 mM Na2HPO4, and 2 mM KH2PO4, pH 7.4). Aliquots were formulated in PBS with 10% glycerol as a cryoprotectant and stored at −80° C. until use.

### 2. Animal Experiments

All mouse experiments were performed under a Rutgers University Institutional Animal Care and Use Committee approved protocol (IACUC Protocol number: PROTO99990006). All hamster experiments were performed in the animal biosafety level-3 (ABSL-3) facilities, following the ethical policies and protocols approved by the Rutgers University Institutional Animal Care and Use Committee (IACUC Approval no. PROTO202000103), which is consistent with the policies of the Association for Assessment and Accreditation of Laboratory Animal Care (AAALAC), the American Veterinary Medical Association (AVMA), the Center for Disease Control (CDC) and the United States Department of Agriculture (USDA).

3. Immunization of BALB cJMice

Cohorts of 5 female, 8-10-week old BALB/c mice were immunized by injection into the gastrocnemius muscle with the indicated amount of MT-001 adjuvanted in 500 μg Alhydrogel® in a final volume of 50 μl. Mice were boosted 21 days later by injection of the same MT-001/Alhydrogel® dose. Where indicated, 20 μg of CpG-ODN1826 was added to the MT-001/Alhydrogel® mix immediately before immunization. Pre-immune sera were collected 3 days prior to the initial immunization, and immune sera were collected after immunizations, as indicated in each related figure.

### 4. RBD-Binding ELISA

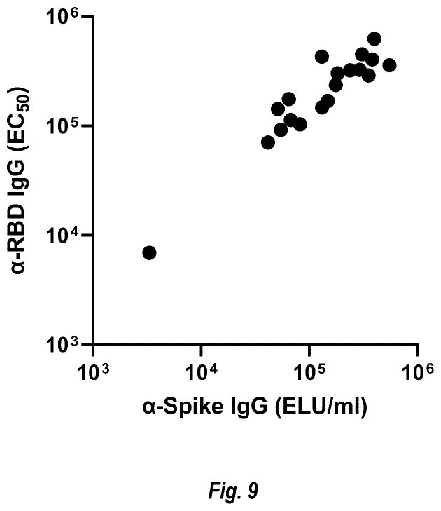

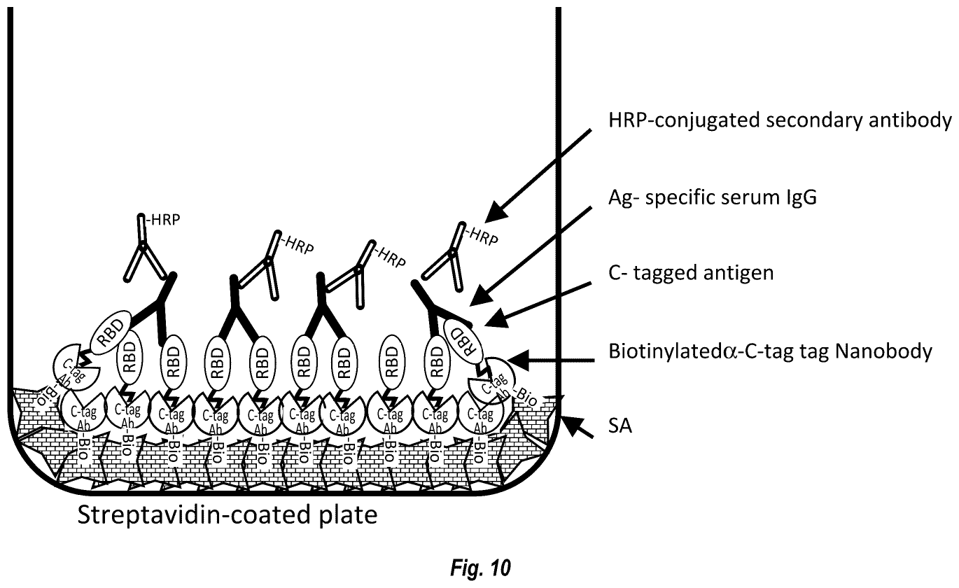

RBD-specific IgG antibody levels were assessed using a novel sandwich ELISA (FIG. 10). This assay was developed to maintain 3D conformational epitopes of RBD and prevent the loss of epitopes that may be denatured by direct adsorption of protein to plastic. Plates were coated with 1 g/ml streptavidin (Sigma, S4762) diluted in PBS and incubated at 4° C. overnight. The next day, plates were washed three times with 0.1% TWEEN-20 in PBS (PBS-T), blocked with PBS-T containing 3% BSA for 1 hour at room temperature and incubated at 4° C. overnight with 1 g/ml biotinylated-camelid α-C-Tag-specific antibody (Thermo Fisher Scientific, #7103252500) in PBS-T. Plates were washed, incubated for 1 hour at room temperature with 5 g/ml MT-001 (containing the C-tag) in PBS-T, washed and incubated at 4° C. overnight with serially diluted mouse serum in blocking buffer. To quantify total IgG levels in the ELISAs, plates were washed and incubated for 1 hour at room temperature with goat anti-mouse IgG horseradish peroxidase (HRP) (Jackson ImmunoResearch, 115-035-166): IgG1 and IgG2a/b levels were quantified using goat anti-mouse IgG1 HRP (Southern Biotechnology Associates, Inc., 1070-05) and goat anti-mouse IgG2a HRP (Southern Biotechnology Associates, Inc., 1080-05) with anti-mouse IgG2b HRP (Southern Biotechnology Associates, Inc., 1090-05), respectively. ELISAs with hamster sera utilized goat anti-Syrian hamster IgG HRP (Jackson ImmunoResearch,107-035-142). All HRP-conjugated secondary antibodies were diluted 1:5,000 in PBS-T. Finally, plates were washed, and 1-Step Ultra TMB-ELISA substrate solution (Thermo, 34029) was added to each well to detect HRP activity. Development was halted by the addition of 1M sulfuric acid and absorbance at 450 nm assessed using a SpectraMax i3 microplate reader. Background values were recorded from wells containing block solution only and subtracted from the raw OD450 values. For ELISAs with mouse serum, a standard curve using titrated SARS-CoV-2 mouse monoclonal antibody (Sino Biological, 40592-MM57) was included on each plate as a technical control to monitor plate-to-plate variability. Data were analyzed in GraphPad Prism using a sigmoidal four-parameter logistic (4PL) fit, and ELISA half-maximal titers were defined as the reciprocal serum dilution that yielded 50% maximal absorbance. ELISAs were repeated at least three times for each of the mouse or hamster serum samples, and the data represent average half-maximal titers for each set of replicates. An independent confirmation of the precision and accuracy of our indirect “sandwich” RBD ELISA method was obtained by submitting a test panel of mouse sera for analysis by Nexelis™ (Laval, Quebec, CA) using a clinically validated SARS-CoV-2 anti-spike IgG ELISA assay (nexelis.com/our-expertise/infectious-diseases/vaccine/sars-cov-2/). Replicate serum samples assayed by the anti-RBD IgG ELISA at Rutgers (above) and, in parallel, an optimized automated anti-spike IgG ELISA at Nexelis™ yielded highly concordant results with quantitatively similar titers (FIG. 9).

### 5. Propagation of SARS-CoV-2

Vero E6 cells were propagated in DMEM containing 10% FBS to 80% confluency in multiple T75 flasks (Corning, CA, USA), and harvested by gentle dissociation of the monolayer with Accutase™ Cell Detachment Solution following the instructions of the manufacturer (Thermo Fisher Scientific, CA, USA). Pooled cells were washed twice in sterile PBS (pH 7.2) and checked for viability by the Trypan Blue (Thermo Fisher Scientific, CA, USA) exclusion method. Cells were seeded into T75 flasks to ˜80% confluency in DMEM containing 10% FBS, and after 18 hours, the spent media was decanted, and the cells were washed with sterile PBS (pH 7.2). The SARS-CoV-2/USA-WA1 strain was diluted in DMEM containing 2% FBS to prepare a multiplicity of infection (MOI) of ˜0.1 for infection. The Vero cells in the T75 flask were infected with 1 ml of virus suspension and incubated at 37° C. for 1 hour, followed by replenishing cells with 10 ml of DMEM containing 2% FBS. The cell culture supernatant containing the virus was harvested at 72 hours post-infection by centrifugation, followed by filtration using a 0.4-micron filter (Millipore-Sigma, MO, USA). Aliquots of virus-containing media (inoculum) were stored at −80° C. until ready to use. Vials of frozen inoculum were randomly selected, thawed, serially diluted in sterile PBS (pH 7.2), and the infectious virus particles were quantitated by plaque assay.

### 6. Virus Inoculum Titration

Virus infectivity was quantitated by plaque assay using Vero E6 cells. Briefly, 4×10⁵ Vero cells/well were seeded onto a six-well cell culture plate (Corning, CA, USA) in DMEM supplemented with L-glutamine and 10% FBS. At 18 hours post-seeding, the cells were washed with sterile PBS (pH 7.2), and 400 μL of 10-fold dilutions (10³ to 10⁷) of the virus, prepared in serum-free DMEM, was added to each well and incubated at 37° C. with gentle rocking of plates every 15 minutes for 1 hour. Then the virus inoculum was carefully removed, and the cells were overlayed with 4 ml/well of 1.6% agarose prepared in DMEM with 4% FBS. The plates were allowed to solidify at room temperature for ˜15 minutes and transferred to a 37° C. incubator with 5% CO2. At 3 days post infection, the plates were fixed with 10% buffered formalin for 30 minutes and washed with sterile PBS (pH 7.2). The agar plugs were gently removed, and the cells were stained with 0.2% crystal violet in 20% ethanol for 10 minutes. The wells were washed with sterile water and dried, and plaques were counted and presented as the number of plaque-forming units (PFU) per gram or ml of tissue or lysate.

### 7. Hamster Infection Studies

Five-to-six-week old male golden Syrian hamsters (Mesocricetus auratus) were procured from Envigo™ and housed in animal biosafety level-2 containment (ABSL-2) for a week to acclimate. One group of hamsters (n=8) was vaccinated with adjuvanted MT-001, and another group of hamsters (n=6), injected with PBS plus Alhydrogel®, served as the control. The MT-001 vaccine or PBS was mixed with Alhydrogel® and incubated for 5 minutes with gentle rocking. Then the MT-001/Alhydrogel® and the PBS/Alhydrogel® mixtures were supplemented with CpG-ODN1826 immediately prior to injection. Each hamster was injected intramuscularly, in the flank, with 50 μL of the respective RBD or control vaccines containing: 10 μg of MT-001 (or an equal volume of PBS), 500 μg of Alhydrogel®, and 100 μg of ODN1826. The hamsters were administered a second dose of MT-001 or PBS control 21 days after the primary dose. Blood from all animals was collected on the day of vaccination (day 0; pre-bleed) and at 14, 21, 28, 35, and 42 days post-vaccination. On day 42, post-primary vaccination, all animals were challenged with SARS-CoV-2/USA-WA1 strain (BEI Resources, VA, USA) at 10⁵ PFU/hamster in 40 μl through intranasal instillation (20 μl/nostril), as previously reported (40). Hamsters were weighed every day following infection and euthanized on day 4 post-infection. Necropsy was performed, and blood and lungs were collected under aseptic conditions.

### 8. Lung Viral Load Assessment

Lung homogenates were prepared using a 0.3 mg (˜40% total lung weight) portion of lung tissues in a screw cap vial containing 1 ml of DMEM media and 0.3 ml (wt/vol) of 1 mm Zirconia/silica beads (MP Biomedicals, LLC, CA, USA). Tissues were lysed by using a FastPrep homogenizer (MP Biomedicals, LLC, CA, USA). The homogenates were centrifuged, and the supernatant was filtered through a 0.45-micron filter, diluted in serum-free DMEM, and 400 μL was used to infect Vero E6 cell monolayers in the six-well plates for a virus plaque assay.

### 9. Determination of Viral Load by Quantitative PCR

Total RNA was extracted from the lungs using TRIzol™ reagent and purified by RNeasy™ mini columns (Qiagen, CA, USA). The eluted RNA was subjected to complementary DNA synthesis using a High-Capacity cDNA Reverse Transcription Kit as per the suggested protocol (Applied Biosystems, CA, USA). Quantitative PCR (qPCR) was performed as described by Ramasamy et al. (40) using SARS-CoV-2 N gene-specific primers (SARS-CoV-2_N-F1: GTGATGCTGCTCTTGCTTTG; SEQ ID NO: 101) and SARS-CoV-2_N-R1: GTGACAGTTTGGCCTTGTTG; SEQ ID NO: 102) and Power SYBR Green PCR MasterMix as per the manufacturer's protocol (Applied Biosystems, CA, USA). The SARS-CoV-2 N gene-specific primers were used to amplify a 97 bp product by conventional PCR and this was purified by the Qiagen gel extraction kit (Qiagen, CA, USA). The purified N gene PCR products were used in real-time PCR to prepare a standard curve. Viral copy numbers in the lung samples were determined from the standard curve.

### 10. Virus Neutralization Assay

The SARS-CoV-2 neutralization assay was performed using the standard protocol described by Ravichandran et al., 2020 (41). Briefly, 100 TCID50 of SARS-CoV-2 isolate USA-WA1/2020 or Omicron variant (B.1.1.529) was added to a two-fold dilution series of serum samples in DMEM containing 10% fetal bovine serum. The serum-virus mixtures were incubated at 37° C. for 1 hour. Meanwhile, a single-cell suspension of Vero E6 cells was prepared in DMEM containing 10% fetal bovine serum at 1.4×10⁴ cells in 20 μl/well in white 96-well flat-bottom Nunc MicroWell plates (ThermoFisher). Following incubation, 100 μL of the serum-virus mixture was added to each well. Additional wells omitting either the serum samples or the virus were included as controls. The plates were gently rocked for the uniform distribution of cells and then incubated for 72 hours at 37° C. with 5% CO2. Plates were equilibrated to room temperature for 30 minutes, after which 50 μL of CellTitre Glo™ reagent (Cat #G7572, Promega, USA) was added to each well, and the plates were gently rocked for 2 minutes and incubated at room temperature for 10 minutes. The luminescence from the wells was measured using Cytation™ 5 Cell Imaging Multi-Mode Reader (BioTek Instruments, VT, USA). The luminescence from blank wells containing 120 μL DMEM with 10% fetal bovine serum and 50 μL CellTitre Glo™ reagent was recorded as baseline values. The 50% neutralization titer (NT50) was calculated using Graph Pad® Prism from a sigmoidal four-parameter logistic (4PL) fit to the luminescence data using the geometric means of the positive and negative controls to bound the top and bottom of the curve.

### 11. Histopathology

The formalin-fixed hamster lung portions were embedded in paraffin and sectioned following standard protocol, as we reported previously (40). The hematoxylin-eosin-stained lung sections were analyzed using the EVOS FL cell imaging system, Thermo Scientific. Pulmonary inflammation was scored according to the severity as follows; 0—no cellular infiltration and intact alveoli, 1—mild cellular infiltration with 1 or 2 foci and intact alveoli, 2—prominent multifocal cellular infiltration with no visible alveoli, 3—significant cellular infiltration involving a larger area of the lung with no visible alveoli, 4—highest cellular infiltration involving extensive area of the lung with no visible alveoli.

### Example 2: Antigen Construct Design Impacts Both the Manufacturability and Immunongenicity of a Protein Component Vaccine

Upon the publication of the ancestral SARS-CoV-2 Wu-1 strain DNA sequence in early 2020 (42), antigen construct design principles were applied to create a well-folded and soluble spike RBD antigen based on a fragment of the S1 subunit. The design of the construct is critical when parsing a multi-domain protein into smaller expressible subunits (43). Previously, over 1000 unique human antigens were provided to the NIH Common Fund Protein Capture Reagents Program for renewable antibody generation (43, 44). Central to this effort was a bioinformatics toolbox, developed by the Northeast Structural Genomics Consortium, for parsing multi-domain proteins into subdomains that could be expressed recombinantly (45). These tools, involving meta-analyses of protein amino acid sequences using various protein structure prediction methods, have been used successfully to design and optimize thousands of protein constructs for NMR and crystallization studies (46) as well as antigens for antibody discovery (43). In all cases, domain boundaries and other sequence features were given special weight, so as not to truncate constructs within ordered regions required for proper folding or presentation of conformational epitopes (28-30).

An immunogen was designed with the aim of enhanced expression yields and optimal manufacturability. By combining bioinformatics predictions from DisMeta (45) with protein homology models and sequence alignments to known structures, clear domain boundaries that separated the RBD region from the surrounding N-terminal and C-terminal regions of the spike protein were identified. A nucleic acid sequence fragment, which encompassed residues 316-594 of the full-length SARS-CoV-2 spike protein, encoded a 278 amino acid polypeptide with two complex subdomains containing non-contiguous N-terminal and C-terminal residues distal to the RBD ACE2 binding region (FIG. 1A; PDB IDs: 7BYR, 7KNE). In addition to the so-called CD1, RBM (receptor binding motif), and CD2 regions (FIGS. 1A and C), this fragment also included the region immediately C-terminal to the RBD, previously termed C-terminal domain 1 (CTD1), and a portion of the so-called “N-terminal domain” of S1 in SARS-CoV (47). A short, four-residue “C-tag” [-EPEA](31) was appended to the C-terminus of the fragment to facilitate efficient purification from cell culture (38), and the resulting construct was termed MT-001. No linkers or protease cleavage sites were included in order to minimize the number of non-native residues in the expressed protein. As the N- and C-termini of the construct are predicted to be located on the face of the protein opposite the RBM (FIGS. 1A-C), it was thought to be unlikely that the short C-tag would sterically hinder desired antibody interactions. The C-tag also provided a convenient site-specific handle for immobilization when used in downstream assays (see ELISA in Methods). Finally, the immunogenicity of the C-tag has been investigated, and no significant anti-C-tag antibody responses have been observed (48). Transient expression of MT-001 with a mammalian cell secretion vector (ATUM, Inc.) in HEK293 suspension culture demonstrated high titers of MT-001, as described in Example 1 (Methods) above. The purified protein was nearly all monomeric, with an apparent molecular weight of 39.4 kDa consistent with what would be expected for a glycosylated protein (calculated unglycosylated MW=31.6 kDa) (FIGS. 7A-B).

### Example 3: MT-001 Induces a Potent and Durable Anti-SARS-CoV-2 RBD Immune Response in BALB/cJ Mice

To explore the immunization dose-response characteristics and measure the durability of elicited antibody levels, an experiment was performed in which two cohorts of five 8- to 10-week-old female BALB/cJ mice were immunized with 1 g, 3 g, or 15 μg of MT-001. The MT-001 immunogen was formulated with 500 μg Alhydrogel® (alum) and administered as two intramuscular (IM) injections at a 3-week interval (FIG. 2A). Sera were collected at 5, 29, and 52 weeks following the primary immunization (FIG. 2A). The highest RBD-specific IgG half maximal geometric mean titers (GMTs) at each timepoint were observed with the 3 μg and 15 μg doses of MT-001 (EC₅₀>10⁵, FIG. 2B). MT-001 at the 3 μg dose exhibited half-maximal ELISA GMTs comparable in potency to reported 2-shot prime/boost immunization results with approved mRNA vaccines and protein component vaccines assayed in the same BALB/cJ mouse system (36, 37, 49, 50). Most notably, there was no significant waning of the MT-001-induced specific anti-RBD antibody levels in the animals between 5 weeks and 52 weeks post-immunization (FIG. 2B). This is in contrast to the mRNA- and most other protein component-based vaccines where protective antibody levels typically wane with a half-life of approximately two months (51, 52).

### Example 4: Addition of a TLR-9 Agonist CPG ODN1826 to the MT-001 Vaccine Mixture Further Increases Antibody Titers and Promotes a More Balanced Immune Response

Since alum-based adjuvants such as Alhydrogel® promote a type 2 inflammatory response (53), the addition of a TLR-9 agonist, CpG ODN1826 (InvivoGen), was tested for whether it would promote a more balanced immune response. Mice immunized with 3 μg MT-001 formulated with 500 μg Alhydrogel® and 20 μg ODN1826 exhibited significantly increased RBD-specific IgG binding titers compared to mice immunized with MT-001 and Alhydrogel® alone by 5 weeks post-primary immunization (ELISA GMTs≈2×10⁶ for mice where CpG was included vs≈3.5×10⁵ when CpG was omitted), and the enhanced response persisted through 47 weeks (FIG. 3B). In addition to significantly higher levels of RBD-specific IgG1 antibodies (FIG. 3C), these mice had robust RBD-specific IgG2a/b titers (FIG. 3D). Thus the average IgG1:IgG2a/b ratio in mice adjuvanted with both Alhydrogel® and ODN1826 was significantly increased (FIG. 3E), indicative of a more balanced Th1/Th2 response, which may strengthen the protective efficacy of MT-001.

### Example 5: MT-001 Protects Syrian 2Olden Hamsters in a SARS-CoV-2 Pulmonary Challenge Model

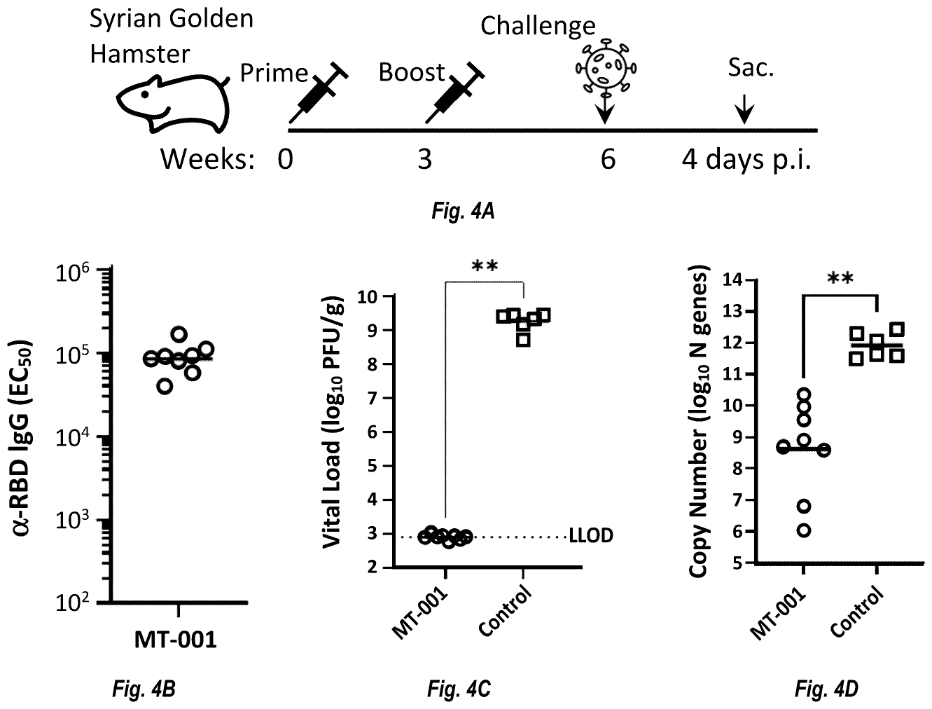

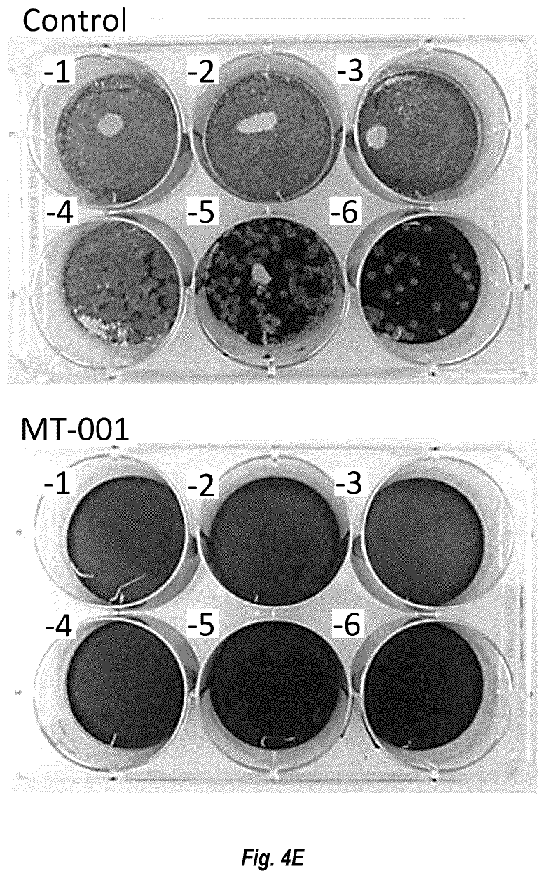

Based on our findings in mice as discussed in Examples 4 and 5 above, MT-001 adjuvanted with Alhydrogel® and ODN1826 was tested for whether the combination would elicit a protective immune response in Syrian golden hamsters. The immune response of these hamsters to SARS-CoV-2 has been shown to recapitulate many of the pathological features seen in humans (54). Hamsters were immunized with 10 μg MT-001 adjuvanted with 500 μg Alhydrogel® and 100 μg ODN1826 and boosted with the same dose 3 weeks later (FIG. 4A). In response, the hamsters had robust RBD-specific IgG titers at six-weeks post-primary immunization (EC₅₀≈10⁵, FIG. 4B). To determine if the antibody response was protective, the vaccinated and control hamster cohorts were next challenged intranasally with 10⁵ PFU of SARS-CoV-2 (USA-WA1 strain) at six-weeks post-primary immunization and monitored for 4 days before analysis. No infectious virus was detected in the lungs of vaccinated hamsters, as evidenced by plaque-forming assays. In contrast, mock-vaccinated control hamsters had a detectable viral load even at a 1:10⁶ dilution of lung homogenates (FIG. 4C). This represented at least a six order of magnitude decrease in infectious virus load in the lungs in the vaccinated hamsters, compared to mock-vaccinated control animals. This difference in viral load, as determined by the PFU assay, was further confirmed by quantitative PCR assessment of lung viral load using SARS-CoV-2 N gene-specific primers. qPCR showed at least a three-log reduction in the total viral RNA burden in the lungs of vaccinated animals (FIG. 4D).

### Example 6: Immunization with MT-001 Produces a Broad Antibody Response Capable of Recognizing and Neutralizing Emergent Variants Including Delta and Omicron

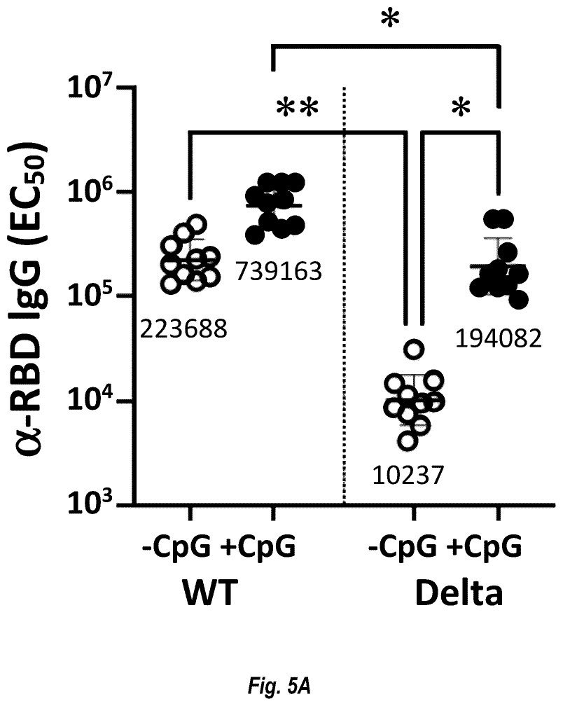

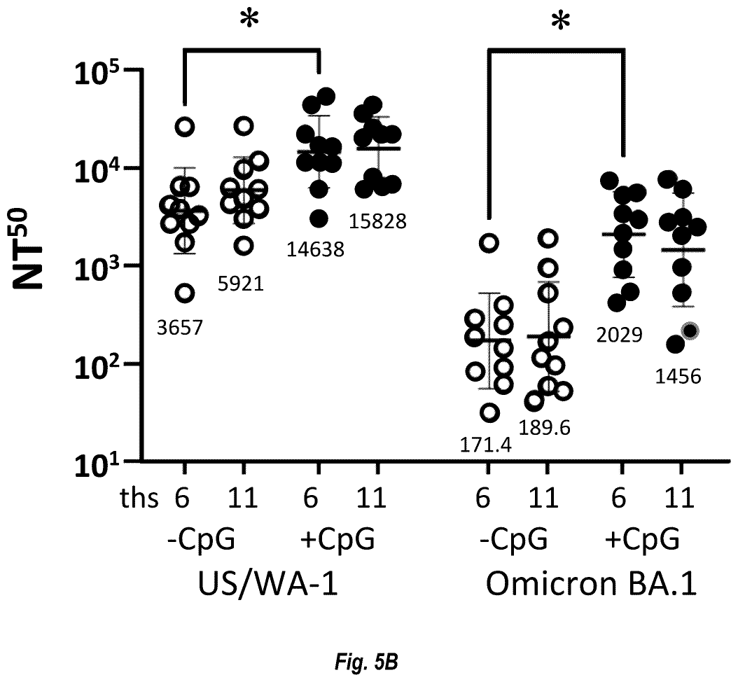

We hypothesized that the enhanced response following immunization with CpG ODN1826 might have also elicited a broader response in mice over time. Therefore, we determined anti-RBD IgG ELISA titers for the ancestral SARS-CoV-2 Wu-1 (“WT”) strain as compared to anti-RBD titers obtained for the SARS-CoV-2 Delta variant (FIG. 5A). For sera obtained at six months post boost from mice vaccinated without CpG, the response to the Delta RBD was more than 20-fold lower than the corresponding response against WT RBD (ELISA GMTs: 10,237 vs. 223,688, respectively). In contrast, sera from mice vaccinated with the inclusion of CpG showed only a 4-fold decrease in titer to Delta RBD when compared to the titers obtained for WT RBD (GMTs: 194,082 vs. 739,163, respectively). These data showed that the inclusion of CpG in the immunization broadened the elicited immune response considerably.

We next tested if the enhanced recognition of the Delta variant RBD in sera from mice vaccinated with the inclusion of CpG correlated with an enhanced ability of the same sera to neutralize the Omicron BA.1 SARS-CoV-2 variant. Neutralization titers to the SARS-CoV-2 Omicron BA.1 strain were markedly increased when CpG was included. On average, sera from mice immunized without CpG had an Omicron BA.1 virus neutralization titer (NT₅₀) of 171 at six months post-boost and 190 at 11 months post-boost; a difference that was not significant. The inclusion of CpG resulted in measured average NT₅₀s of 2092 at six months post-boost and 1456 at 11 months post-boost. Thus, the inclusion of CpG served to increase the neutralization titer against the Omicron variant by 7- to 12-fold compared to Alhydrogel® alone (FIG. 5B). Strikingly, the observed Omicron BA.1 neutralization titer obtained with MT-001 was comparable to neutralizing titers reported in BALB/c mice immunized twice with a variant-matched vaccine, mRNA-1273.529 (55). These data indicate that MT-001 was efficacious in generating nAb responses against emergent variants, despite being based on the ancestral SARS-CoV-2 sequence, and that these nAb responses were durable for at least 11 months.

### DISCUSSION

Designing an expression construct that incorporates a fragment of the SARS-CoV-2 spike protein, encompassing both the spike ACE2 receptor binding motif (RBM) as well as surrounding sequences that provide the local structural context, is not a straightforward task (30). Ideally, the design should result in good expression yields of a relatively “well-behaved” (i.e., stable, well-folded, and soluble) gene product while maximizing antigen immunogenicity and preserving conserved regions that might serve as targets for broadly neutralizing antibodies. Our MT-001 RBD expression construct (FIGS. 1A and C) which includes the spike protein RBM (residues 437-508) together with upstream and downstream regions (residues 316-436 and residues 509-594), encodes a section of the spike protein with an extended polypeptide architecture that appears to be composed of three distinct domain-like regions (FIGS. 1A and C). The term “domain-like” in this sense refers to compact, structurally contiguous subdomain regions of the protein that may have distinct structural and/or functional roles. For example, ACE2 receptor binding is carried out by the domain-like RBM (56). The extended three subdomain structure is tethered at its N- and C-termini by residues S316 and G594 in close proximity, forming the ends of a short antiparallel beta sheet (FIGS. 1A and C). At several points within some of the subdomains, residues relatively distant in the primary sequence form significant interactions in the secondary and tertiary structure. For example, in the central subdomain domain (FIGS. 1A and C), which consists of a twisted five-stranded antiparallel beta sheet composed mostly of CD1 amino acids, the center beta strand (FIGS. 1A and C) comes from the CD2 region C-terminal to the RBM. Also, the CTD1 subdomain (the region from 528-594, FIGS. 1A and C) in our construct forms a well-defined structure, stabilized by the 538-590 disulfide bond (FIG. 1A, arrow), and packs against the N-terminal region (“NT”, 316-332) of the MT-001 spike fragment (FIGS. 1A-B).

These interactions are likely to play an important role in proper folding of the RBD; shorter constructs, involving truncations that result in the loss of these interactions, might partially destabilize the native structure, and could even introduce non-native conformational epitopes. This domain architecture suggests that the spike S1 region represented by the MT-001 RBD construct, spanning amino acids 316-594, may have evolved via two consecutive nested domain insertion events (57, 58). The entire 316-594 region of the spike protein may have undergone selection as a coherent structural and functional unit involved in the conformational transition between the “RBD-down” and “RBD-up” states of the prefusion spike trimer (59). Thus, for an RBD-centric immunogen in a SARS-CoV-2 vaccine, the MT-001 construct is arguably close to the optimal choice. Furthermore, structural analysis of the full-length spike protein shows that CTD1 may act as a relay between the RBD and the fusion-peptide proximal region (FPPR) domains to trigger fusion upon receptor binding (60). The proposed relay function of CTD1 suggests that some antibodies targeting this region might interfere with viral entry and thus have SARS-CoV-2 neutralization activity. Since this region is relatively conserved among sarbecoviruses (FIG. 11) and contains few mutations found in SARS-CoV-2 variants of concern (VOCs), it may be able to elicit broadly neutralizing antibodies (bnAbs) ((61, 62); FIG. 1C).

The properties that make an antigen well suited for expression and purification may also translate into improved immunogenicity in the context of vaccines. By providing an “extended” RBD-containing SARS-CoV-2 spike protein construct that is stable and well-folded without requiring non-native modifications to the sequence, the immune response can be focused on a critical region of the spike protein containing many neutralizing epitopes (22, 62, 63). This strategy may, in addition, minimize decoy or immunodominant epitopes, steric hindrance, or possibly immune suppressive components of the full-length protein. Outbreaks of SARS-CoV and MERS-CoV earlier this millennia, combined with the periodic emergence of new SARS-CoV-2 variants of concern and the constant threat of future coronavirus pandemics, motivates the development of a broadly protective pan-coronavirus vaccine. Within the spike protein, the RBM shares low sequence homology across the coronavirus family, which is expected given the numerous hosts and range of cellular receptors targeted. However, some regions flanking the RBM are relatively highly conserved, especially within the CTD1 subdomain (FIGS. 1A and C, and 11). For an S1 sub-fragment RBD-based vaccine (26), inclusion of the CTD1 subdomain should present additional conserved B-cell and T-cell epitopes which may provide a broader pan-coronavirus response compared to RBD constructs where this region has been truncated. Indeed, a recent study involving a hierarchical Bayesian regression model trained on more than 6 million SARS-CoV-2 genome sequences predicted that even for future, yet to emerge, variants-of-concern (VOC), mutations in the CTD1 subdomain were likely to be relatively rare due to their negative contributions to overall viral fitness (64).

The fusion of purification tags and other non-native sequences must also be considered when designing a vaccine construct. MT-001 uses the C-tag, a short, four residue (-Glu-Pro-Glu-Ala) tag incorporated at the C-terminus of the construct, to enable efficient purification and site-specific immobilization for use in downstream assays (65). This provides several advantages over other commonly used purification tags. Due to its size, the C-tag would be expected to have minimal effect on protein expression and solubility, and tag cleavage may not be required. Studies have found the C-tag itself to be minimally immunogenic, and it has been used successfully in GMP processes for vaccine manufacturing (48). Purification step yields are high after only a single tag-specific affinity chromatography step and, unlike RBD constructs incorporating fusions to non-viral scaffolding moieties (30, 32), nearly 100% of the expressed protein consists of the target SARS-CoV-2 antigen (FIGS. 7A-B). The C-tag allows for indirect solid phase immobilization of the antigen (e.g., in microtiter plate wells) and, with the tag being located on the opposite side of the antigen from the receptor binding motif, allows for unimpeded display of the native 3D antigen structure and efficient capture of antibodies recognizing discontinuous conformational B-cell epitopes of the RBD.

The animal experiments shown here demonstrate that immunization with MT-001 markedly enhanced the production of IgG antibodies specific to SARS-CoV-2 spike protein, with levels comparable to the most effective vaccines characterized in the literature to date (36, 37, 49). In mice, following a two-dose immunization with as little as 1 μg MT-001 adjuvanted with Alhydrogel®, high anti-RBD (FIGS. 2A-B) and anti-spike IgG (FIG. 9) titers were observed. These were associated with the increased production of neutralizing antibodies against both pseudovirus (not shown) and infectious virus (FIGS. 5A-B). Moreover, as demonstrated in two independent experiments, these immune responses were remarkably durable, with minimal waning in antibody titers observed between 5 weeks and one-year post-primary immunization (FIGS. 2A-B and 3A-E). This is in stark contrast to the widely used mRNA vaccines, where antibody titers decay significantly after 6 months (66, 67). Considering the lack of durability observed for most COVID-19 vaccines to date, results from our long-term in vivo studies further differentiate MT-001 from other immunization strategies directed against SARS-CoV-2 (See FIG. 1 of (68)). Durable immunity conferred by vaccines has been attributed to the generation of long-lived plasma cells (LLPCs) residing in bone marrow, which in some cases can express and secrete protective, pathogen-specific antibodies for decades (69, 70). It is possible that MT-001 is unusually capable, especially compared to other SARS-CoV-2 vaccines (70), of eliciting high levels of spike protein-specific LLPCs. However, further studies will be required to address this question at the mechanistic level.

Alum-based adjuvants such as Alhydrogel® are known to primarily elicit a type 2 inflammatory response (71), which may not be ideal for inducing protective immunity against pathogens (72). Previously, CpG containing oligonucleotides have been shown to induce a type 1 response by acting as Toll-like receptor 9 (TLR9) agonists, providing a more balanced Th1/Th2 response when used in conjunction with alum adjuvants (28, 53, 73). Mice immunized with 3 μg MT-001 adjuvanted with Alhydrogel® and the TLR9 agonist CpG ODN1826 exhibited significantly higher anti-RBD IgG titers at 5 weeks post-primary immunization compared to mice immunized with 3 μg MT-001 adjuvanted with Alhydrogel® alone. This increased titer appeared to be partly due to a two order of magnitude increase in the anti-RBD IgG2a/b titers in CpG adjuvanted mice, which resulted in a more balanced IgG2a/b to IgG1 ratio. This is consistent with data reported for another RBD-based protein vaccine utilizing a different CpG oligonucleotide and alum as adjuvants (73). The significantly increased IgG2a/b titer associated with the CpG adjuvant persisted for at least 29 weeks post-primary immunization and was correlated with increased neutralization titers against both the Wu-1 strain and variants of SARS-CoV-2 (FIGS. 3A-E and 5A-B; and see below).

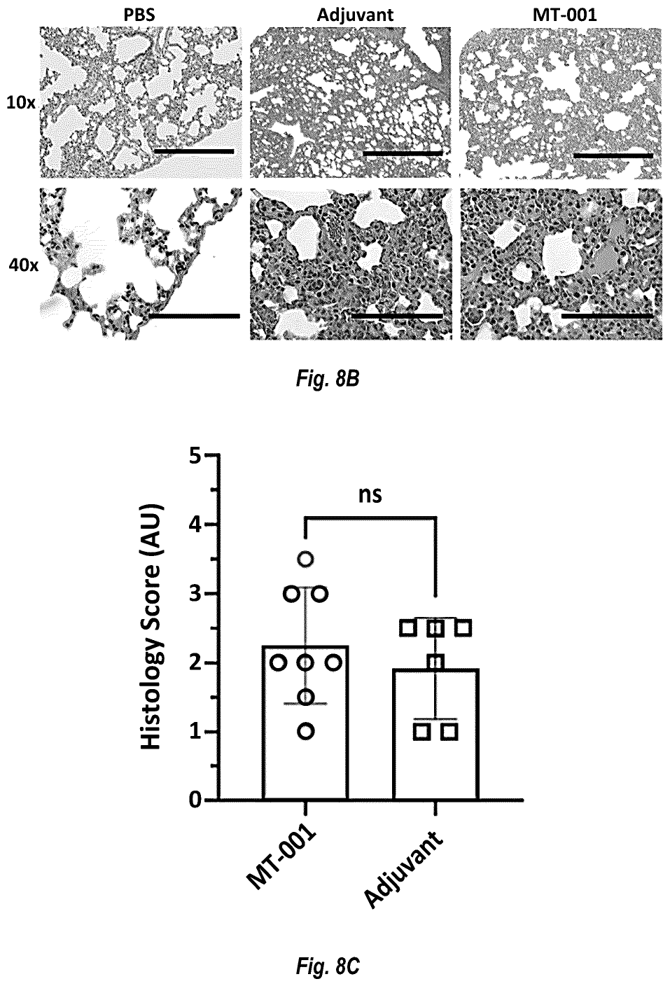

Syrian hamsters are an accepted in vivo model for human SARS-CoV-2 infection as they mimic many of the characteristics of human COVID-19 (54). Therefore, to test the protective efficacy of MT-001 against SARS-CoV-2 infection in vivo, Syrian hamsters were vaccinated and boosted with MT-001 co-adjuvanted with Alhydrogel® plus CpG ODN1826 using the same schedule described above for mice (FIG. 4A). Control animals were vaccinated with the adjuvant only (Alhydrogel® plus CpG ODN1826). The level of infectious SARS-CoV-2 in lung homogenates from MT-001 vaccinated hamsters was undetectable by plaque assay even at the lowest dilution (1/10) of the sample used (FIG. 4E). Therefore, the viral load per gram of lungs in these animals was calculated based on the lower limit of detection of the assay. In the group of mock-vaccinated hamsters, infectious virus plaques were detected even at a 1:10⁶ dilution of lung homogenates. Thus, the viral load/gram of lung tissue was significantly lower (less than or equal to 103 PFU) in MT-001 vaccinated hamsters than in mock-vaccinated hamsters (109 PFU) (FIG. 4C). Likewise, N gene copy numbers as determined by qPCR were on average 1000-fold lower in MT-001 vaccinated hamsters compared to hamsters that received adjuvant alone (FIG. 4D). While weight loss and lung pathology are usually associated with SARS-CoV-2 infection in hamsters, at the viral dose used in this study there were no apparent differences in these two parameters up to 4 days post infection when the hamsters were sacrificed (FIGS. 8A-C), despite the significant reduction in viral burden observed in hamsters vaccinated with MT-001 compared to those that received adjuvant alone. This is consistent with other hamster SARS-CoV-2 challenge experiments in the literature, where the lung pathology between vaccinated and unvaccinated hamsters does not begin to differ until four to six days post-challenge (74, 75). In this case, the observed pathology at these early time points may be driven by the host inflammatory immune response to the adjuvants and/or to the virus as the animal attempts to control viral replication and clear the infection. Further experiments examining pathology and body weight changes at longer durations post infection will be required to characterize the differences in vaccine-induced immune response post-challenge. Collectively, these studies show that vaccination with MT-001 protected hamsters from SARS-CoV-2 infection.

CpG ODN1826, when used as a co-adjuvant with Alhydrogel®, has previously been shown to enhance peak immunogenicity in mice and hamsters with RBD-based SARS-CoV-2 vaccines (73). As discussed herein, in addition to enhancing the potency and Th1/Th2 balance of the immune response elicited by MT-001 (FIGS. 3A-E), inclusion of CpG ODN1826 also enhanced the antibody response to emerging variants (FIGS. 5A-B). This could have been due to a direct enhancement of the breadth of the response (see below) or simply due to a mass action effect where the levels of pre-existing anti-variant antibodies were elevated above a threshold concentration in the serum where they were detectable by the assays used. Further work will be required to determine the mechanistic details underlying CpG augmentation of the immune response in this system. And unlike the waning of immunity against variants seen with spike-based mRNA vaccines, a two-dose regimen of MT-001 elicits a diverse and protective antibody response that persists for at least eleven months. In one recently described experiment, BALB/c mice pre-immunized with two doses of BNT162b2 and boosted on day 104 with the same vaccine, the peak post-boost neutralization titer against Omicron BA.1 was reported to be 2075 GMT (BioNTech Innovation Series Presentation, Jun. 29, 2022). The present disclosure discusses a similar Omicron BA.1 virus neutralization titer (GMT 2092) in BALB/c mice immunized with two doses of MT-001 at six months post-immunization without an additional booster dose (FIGS. 5A-B). In the absence of a booster, mRNA-vaccinated BALB/c mice typically show no detectable variant neutralization titers at a comparable time interval post-immunization (56).

The increased breadth of the immune response observed when the immunogen is adjuvanted with both alum and CpG ODN1826 comports with data showing that TLR-9 agonists activate the innate immune system by signaling through IRF7 while also directly stimulating B cells and dendritic cells (76). This is consistent with the view that adjuvants such as TLR agonists (perhaps necessarily in presence of a co-adjuvant like alum) promote B cell maturation in germinal centers, leading to higher affinity and broader antibody repertoires (76). It has further been suggested that imprinting by innate signals during vaccination, dependent on the type and structure of the immunogen, the adjuvant(s), and the mode of delivery, among other variables, may drive the durability of the immune response by promoting the creation of long-lived plasma cells in bone marrow (68). In translational studies aimed at developing a human vaccine, caution must be exercised when interpreting results with TLR-9 agonists. Compared to mice, humans and other primates express TLR-9 in a more limited subset of immune cell types, chiefly plasmacytoid dendritic cells and B cells (77). However, it is reassuring that, for at least one other RBD-based SARS-CoV-2 vaccine (“RBD-I53-50”), a careful comparison of results with alum plus a TLR-9 co-adjuvant (CpG-1018) in both mice (strain C57BL/6) and NHPs (rhesus macaques) has been published (78-80). It is noteworthy that for a number of key immunological metrics, including peak neutralizing antibody titers against the parent SARS-CoV-2 strain, neutralizing antibody titers against variants, CD4 T cell responses, Th1 cytokine responses, and protection in a virus challenge assay, comparable responses were observed for the RBD-I53-50 vaccine, co-adjuvanted with alum and CpG-1018, in both mice and nonhuman primates. This concordance is encouraging and suggests that the results presented here for MT-001 will have predictive value going forward in translational preclinical and clinical studies.

Regarding the translational relevance of the preclinical animal data presented here to future expectations for a human vaccine—particularly with respect to the durability of the immune response—attention should be paid to recent Phase 2 clinical data presented for the Corbevax™ vaccine (81). Corbevax™, like MT-001, incorporates an RBD-based immunogen, although the construct used to express the antigen for Corbevax™, compared to the MT-001 design, is truncated at both the N- and C-termini (332-549) and modified to remove an unpaired cysteine (C538 Å) (27, 28). Corbevax™ is also produced in yeast cells rather than animal cells like MT-001. However, like MT-001 Corbevax™ is co-adjuvanted with alum and a CpG TLR-9 agonist (CpG ODN1826 in mice; CpG1018 in humans). In BALB/c mouse studies, Corbevax™, when adjuvanted with alum alone, exhibits only modest IgG titers and pseudovirus neutralization titers (82); hence clinical studies with this vaccine have focused exclusively on formulations incorporating both the alum and the CpG adjuvants. Recently-published Phase 2 studies of Corbevax™ have shown that it, like MT-001, exhibits remarkable durability up to 12 months post vaccination (81). Another notable SARS-CoV-2 vaccine for comparison purposes is the SCB-2019 vaccine developed by Clover Biopharmaceuticals. The SCB-2019 immunogen is the full-length spike protein ectodomain (based on residues 1-1211 of the ancestral Wuhan-HU-1 strain), trimerized via a proprietary C-terminal tag derived from human collagen (50). Like Corbevax™, SCB-2019 is adjuvanted with alum and CpG1018. When used to immunize female BALB/c mice at a 3 μg dose with a simple prime/boost regimen three weeks apart, SCB-2019 exhibited excellent persistence of the antibody broad neutralization titers after 140 days (FIG. 4C in reference 83). However, compared to durable MT-001 live virus neutralization titers after 6 months of approximately 2000 GMT against Omicron BA.1.1.529 (FIG. 5B, present disclosure), the SCB-2019-immunized mice, without a third dose, exhibited Omicron BA.1.1.529 pseudovirus neutralization titers of <100 GMT) at all the later time points (FIGS. 4C and Table 2 [“No 3^{rd} dose boost Control”] in reference 83).

Taken together, the above results strongly suggest that the vaccine durability results presented here for MT-001 in mice will translate to humans. Moreover, our results show, at least for the MT-001 construct studied, that the aluminum hydroxide adjuvant alone, without the CpG TLR-9 agonist co-adjuvant, is sufficient to endow the vaccine humoral immune response with the property of high durability (FIGS. 2A-B and 3A-E). Thirdly, it is interesting that the Omicron neutralizing antibody titers elicited by MT-001 are significantly higher (>20-fold) than those elicited by SCB-2019, even though the spike fragment sequence of MT-001 is entirely contained within the sequence of the SCB-2019 spike ectodomain sequence. Unless the higher anti-Omicron titers observed in mice for MT-001 vs. SCB-2019 are due to differences in the respective neutralization assay protocols (e.g., live virus assays for MT-001 vs. pseudovirus assays for SCB-2019), it suggests that MT-001 may display to the immune system cryptic B cell epitopes that are buried in the 3D structure of the full-length, trimeric spike ectodomain holoprotein—and thus less available for neutralizing antibody elicitation.

MT-001 was designed from inception for improved manufacturability using construct design techniques refined during operation of a high-throughput human protein production pipeline (43, 44). High yield streamlined GMP manufacturing using standard protocols and existing infrastructure widely available in the biotech and pharmaceutical industry (e.g., 2000-L bioreactors, production-scale protein purification systems, know-how and associated ancillary equipment) should facilitate large-scale, cost-effective production of MT-001. This, combined with MT-001's potent and durable immunogenicity, ability to neutralize emerging variants, and its biophysical properties and reduced logistical requirements for wide-spread distribution, make it an attractive candidate for further development on a global scale.

The potency, durability, and protection from variants afforded by MT-001 suggest it may also be an ideal vaccine, perhaps as a booster subsequent to immunization with an mRNA-based vaccine, to meet the needs of people who are immunocompromised and are not adequately served by existing COVID-19 vaccines. Such patients constitute one of the groups with the highest risk of a severe outcome following SARS-CoV-2 infection (84, 85). Although a strong CD8 T-cell response is observed in approximately two-thirds of patients receiving anti-CD20 immunotherapy for lymphoma, humoral immune responses in such patients respond poorly to existing vaccines due to their depleted B cell population (86). However, a SARS-CoV-2 vaccination several weeks prior to the start of anti-CD20 treatment can effectively elicit antibodies that appear to be at least temporarily protective (87). With currently available SARS-CoV-2 vaccines exhibiting waning immunity and thus requiring repeat booster doses for continued protection (70), subsequent booster shots may thus be ineffective. Instead, a COVID-19 vaccine like MT-001 which provides durable anti-SARS-CoV-2 antibody levels may confer unbroken SARS-CoV-2 protection for the entire course of anti-CD20 immunotherapy, assuming vaccination could be provided prior to the start of B cell ablation.

Finally, even individuals who are not immunocompromised may benefit from the safety and peace of mind conferred by a COVID-19 vaccine that does not necessitate frequent booster doses to maintain protective levels of anti-SARS-CoV-2 antibodies. The well-grounded anxiety surrounding the uncertainty of whether one's immunity has waned enough to merit another booster shot is heightened by some recent large scale retrospective studies showing that re-infections —even among people who are fully-vaccinated and boosted—increases the risk of hospitalization for multi-organ system involvement and acute and post-acute sequelae that in some cases lead to death (88). Failure to adequately tamp down SARS-CoV-2 spread also endangers society in general by promoting conditions that enable the emergence of new variants of concern. For all of these reasons the continued development of durable, transmission-blocking COVID-19 vaccines remains a goal of paramount importance. Vaccines with durable immunity may also facilitate the establishment of threshold correlates of protection (CoPs), based on neutralizing antibody titers, because non-waning antibody levels should enable more accurate projections of vaccine efficacy in the post-vaccination period (89). Established CoPs would provide valuable guidance to regulatory agencies seeking to accelerate the process of approval for COVID-19 vaccines aimed at protecting diverse populations. The SARS-CoV-2 pandemic enveloped the entire world in the last three years and, as the virus continues to evolve and new variants emerge, medical countermeasures are still playing catch-up. Vaccines such as MT-001 could be in the vanguard of a future toolkit of impactful new vaccines and therapies that offer the promise of a globally coherent solution.

### Example 7: Comparison of Immune Response to MAV-004 and MT-001

The immune response in mice to MT-001 was also compared against the immune response to a mixture of SARS-CoV-2 constructs called MAV-004, based on the P0DTC2 sequence positions 316-594 (SEQ ID NO: 3). Specifically, MAV-004 comprises an equimolar mixture of a wild-type RBD (comprising SEQ IDNO: 3; and thus corresponding to MT-001, which comprises SEQ ID NO: 4 herein), and three different mutants, (1) comprising SEQ ID NO: 3 but with a Y365L substitution, (2) comprising SEQ ID NO: 3 but with a V511A substitution, and (3) comprising SEQ ID NO: 3 but with an I402V substitution.

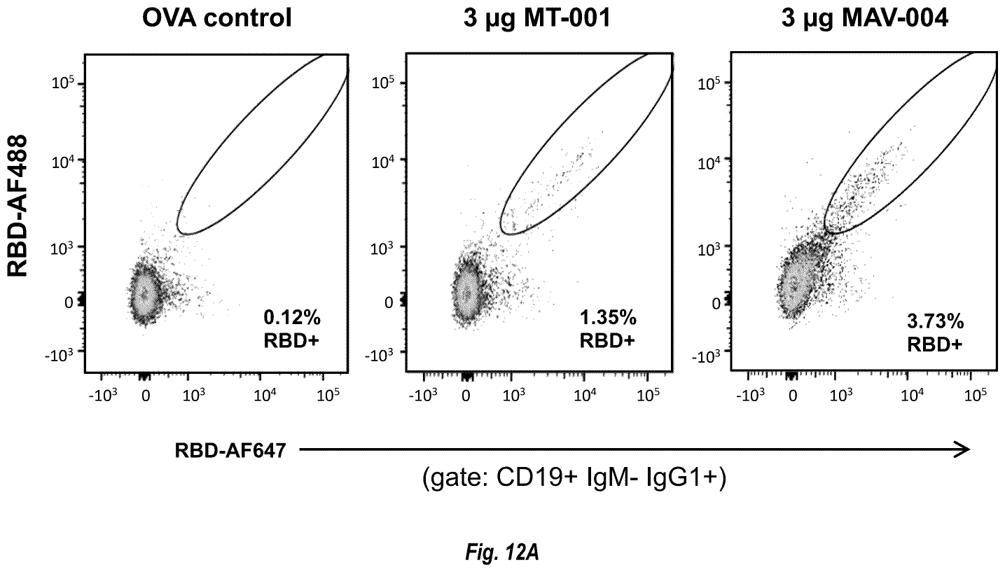

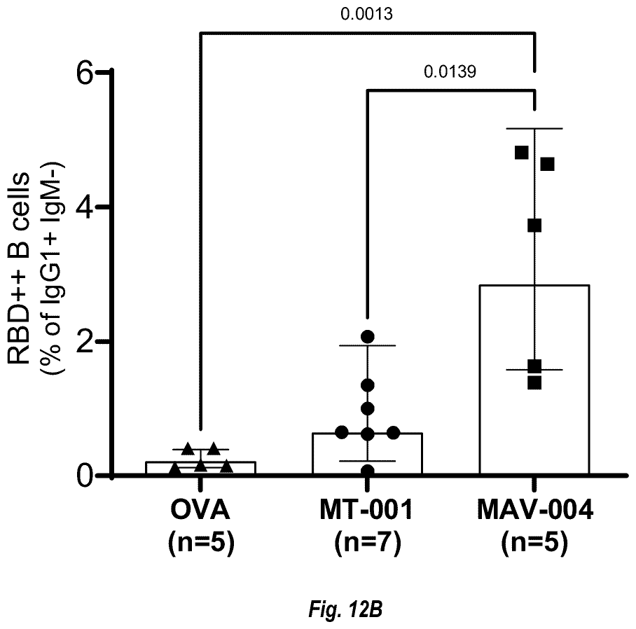

A 3 μg dose of each of an MT-001 and an MAV-004 vaccine was administered to mice, along with a control (OVA). There were 5 mice in the control and MAV-004 group and 7 mice in the MT-001 group. Representative flow cytometry results (shown in FIG. 12A) show comparison of a 3 μg dose of MT-001 to a 3 μg dose of the mixture MAV-004. Specifically, the percentage of isotype switched splenic wild-type RBD positive B cells (CD19+, IgM+, IgG1+) was higher with MAV-004 than with MT-001. The wild-type RBD positive B cells (CD19+, IgM+, IgG1+) percentage was determined by double labeling with wild-type RBD-A488 and wild-type RBD-A647 at 55 days after boosting with MT-001 or MAV-004. As shown in FIG. 12B, vaccination with MAV-004 results in a significantly increased (3.5 fold) fraction of circulating RBD-specific memory B-cells compared to MT-001 (one-way ANOVA, Tukey's multiple comparison, P<0.05).

### Example 8: Vaccine Constructs MT-002 and MT-003 Against Omicron and XBB 1.5 Strains

Two further vaccine constructs were prepared. MT-002 comprises residues 316-594 of SARS-CoV-2 strain BA.5 (as shown in SEQ ID NO: 5), fused to a C-terminal C-tag of sequence EPEA (SEQ ID NO: 103), thus corresponding to SEQ ID NO: 6. MT-003 comprises residues 316-594 of SARS-CoV-2 strain XBB 1.5 (as shown in SEQ ID NO: 77), fused to a C-terminal C-tag of sequence EPEA (SEQ ID NO: 103), thus corresponding to SEQ ID NO: 78. The MT-002 and MT-003 constructs were codon-optimized and expressed via a secretion vector in HEK293 cells by ATUM, Inc., Newark, CA, and purified in a single affinity chromatography step using the CaptureSelect™ C-tagXL system (ThermoFisher) (38, 39). Aliquots were formulated in PBS with 10% glycerol as a cryoprotectant and stored at −80° C. until use.

The final purified yield of MT-003 was relatively high compared to similar constructs, specifically approximately 355 mg from 1 L suspension culture. The purified protein was >96% monomeric with an apparent molecular weight of 38.09 kDa (calculated 31.6 kDa) by HPLC-SEC).

### REFERENCES

1. Baden L R, El Sahly H M, Essink B, Kotloff K, Frey S, Novak R, Diemert D, Spector S A, Rouphael N, Creech C B, McGettigan J, Khetan S, Segall N, Solis J, Brosz A, Fierro C, Schwartz H, Neuzil K, Corey L, Gilbert P, Janes H, Follmann D, Marovich M, Mascola J, Polakowski L, Ledgerwood J, Graham B S, Bennett H, Pajon R, Knightly C, Leav B, Deng W, Zhou H, Han S, Ivarsson M, Miller J, Zaks T. Efficacy and Safety of the mRNA-1273 SARS-CoV-2 Vaccine. New England Journal of Medicine. 2020; 384(5):403-16. doi: 10.1056/NEJMoa2035389. PubMed PMID: 33378609.
2. Polack F P, Thomas S J, Kitchin N, Absalon J, Gurtman A, Lockhart S, Perez J L, Perez Marc G, Moreira E D, Zerbini C, Bailey R, Swanson K A, Roychoudhury S, Koury K, Li P, Kalina W V, Cooper D, Frenck R W, Hammitt L L, Tureci Ö, Nell H, Schaefer A, Unal S, Tresnan D B, Mather S, Dormitzer P R, Sahin U, Jansen K U, Gruber W C. Safety and Efficacy of the BNT162b2 mRNA Covid-19 Vaccine. New England Journal of Medicine. 2020; 383(27):2603-15. doi: 10.1056/NEJMoa2034577. PubMed PMID: 33301246.
3. Hodcroft E B. “CoVariants: SARS-CoV-2 Mutations and Variants of Interest.” 2021: covariants.org/.
4. Harvey W T, Carabelli A M, Jackson B, Gupta R K, Thomson E C, Harrison E M, Ludden C, Reeve R, Rambaut A, Peacock S J, Robertson D L, Consortium C-GU. SARS-CoV-2 variants, spike mutations and immune escape. Nature Reviews Microbiology. 2021; 19(7):409-24. doi: 10.1038/s41579-021-00573-0.
5. Morens D M, Taubenberger J K, Fauci A S. Universal Coronavirus Vaccines An Urgent Need. New England Journal of Medicine. 2021; 386(4):297-9. doi: 10.1056/NEJMp2118468.
6. Nohynek H, Wilder-Smith A. Does the World Still Need New Covid-19 Vaccines?New England Journal of Medicine. 2022; 386(22):2140-2. doi: 10.1056/NEJMe2204695.
7. Sette A, Crotty S. Immunological memory to SARS-CoV-2 infection and COVID-19 vaccines. Immunological Reviews.n/a(n/a). doi: doi.org/10.1111/imr.13089.
8. Rosenbaum L. Escaping Catch-22 Overcoming Covid Vaccine Hesitancy. New England Journal of Medicine. 2021; 384(14):1367-71. doi: 10.1056/NEJMms2101220. PubMed PMID: 33577150.
9. Gagne M, Moliva J I, Foulds K E, Andrew S F, Flynn B J, Werner A P, Wagner D A, Teng I T, Lin B C, Moore C, Jean-Baptiste N, Carroll R, Foster S L, Patel M, Ellis M, Edara V V, Maldonado N V, Minai M, McCormick L, Honeycutt C C, Nagata B M, Bock K W, Dulan C N M, Cordon J, Flebbe D R, Todd J M, McCarthy E, Pessaint L, Van Ry A, Narvaez B, Valentin D, Cook A, Dodson A, Steingrebe K, Nurmukhambetova S T, Godbole S, Henry A R, Laboune F, Roberts-Torres J, Lorang C G, Amin S, Trost J, Naisan M, Basappa M, Willis J, Wang L, Shi W, Doria-Rose N A, Zhang Y, Yang E S, Leung K, O'Dell S, Schmidt S D, Olia A S, Liu C, Harris D R, Chuang G Y, Stewart-Jones G, Renzi I, Lai Y T, Malinowski A, Wu K, Mascola J R, Carfi A, Kwong P D, Edwards D K, Lewis M G, Andersen H, Corbett K S, Nason M C, McDermott A B, Suthar M S, Moore I N, Roederer M, Sullivan N J, Douek D C, Seder R A. mRNA-1273 or mRNA-Omicron boost in vaccinated macaques elicits similar B cell expansion, neutralizing responses, and protection from Omicron. Cell. 2022; 185(9):1556-71.e18. Epub 2022/04/22. doi: 10.1016/j.cell.2022.03.038. PubMed PMID: 35447072; PMCID: PMC8947944.
10. Ying B, Scheaffer S M, Whitener B, Liang C Y, Dmytrenko O, Mackin S, Wu K, Lee D, Avena L E, Chong Z, Case J B, Ma L, Kim T, Sein C, Woods A, Berrueta D M, Carfi A, Elbashir S M, Edwards D K, Thackray L B, Diamond M S. Boosting with Omicron-matched or historical mRNA vaccines increases neutralizing antibody responses and protection against B.1.1.529 infection in mice. bioRxiv. 2022. Epub 2022/02/17. doi: 10.1101/2022.02.07.479419. PubMed PMID: 35169795; PMCID: PMC8845417.
11. Walsh E E, Frenck R W, Falsey A R, Kitchin N, Absalon J, Gurtman A, Lockhart S, Neuzil K, Mulligan M J, Bailey R, Swanson K A, Li P, Koury K, Kalina W, Cooper D, Fontes-Garfias C, Shi P-Y, Tureci Ö, Tompkins K R, Lyke K E, Raabe V, Dormitzer P R, Jansen K U, Sahin U, Gruber W C. Safety and Immunogenicity of Two RNA-Based Covid-19 Vaccine Candidates. New England Journal of Medicine. 2020; 383(25):2439-50. doi: 10.1056/NEJMoa2027906.
12. Zhang Z, Mateus J, Coelho C H, Dan J M, Moderbacher C R, Gálvez RI, Cortes F H, Grifoni A, Tarke A, Chang J, Escarrega E A, Kim C, Goodwin B, Bloom N I, Frazier A, Weiskopf D, Sette A, Crotty S. Humoral and cellular immune memory to four COVID-19 vaccines. bioRxiv. 2022:2022.03.18.484953. doi: 10.1101/2022.03.18.484953.
13. Bar-On Y M, Goldberg Y, Mandel M, Bodenheimer O, Amir O, Freedman L, Alroy-Preis S, Ash N, Huppert A, Milo R. Protection by a Fourth Dose of BNT162b2 against Omicron in Israel. New England Journal of Medicine. 2022; 386(18):1712-20. doi: 10.1056/NEJMoa2201570.
14. Regev-Yochay G, Gonen T, Gilboa M, Mandelboim M, Indenbaum V, Amit S, Meltzer L, Asraf K, Cohen C, Fluss R, Biber A, Nemet I, Kliker L, Joseph G, Doolman R, Mendelson E, Freedman L S, Harats D, Kreiss Y, Lustig Y. 4th Dose COVID mRNA Vaccines' Immunogenicity &amp; Efficacy Against Omicron VOC. medRxiv. 2022:2022.02.15.22270948. doi: 10.1101/2022.02.15.22270948.
15. Falsey A R, Sobieszczyk M E, Hirsch I, Sproule S, Robb M L, Corey L, Neuzil K M, Hahn W, Hunt J, Mulligan M J, McEvoy C, DeJesus E, Hassman M, Little S J, Pahud B A, Durbin A, Pickrell P, Daar E S, Bush L, Solis J, Carr Q O, Oyedele T, Buchbinder S, Cowden J, Vargas S L, Guerreros Benavides A, Call R, Keefer M C, Kirkpatrick B D, Pullman J, Tong T, Brewinski Isaacs M, Benkeser D, Janes H E, Nason M C, Green J A, Kelly E J, Maaske J, Mueller N, Shoemaker K, Takas T, Marshall R P, Pangalos M N, Villafana T, Gonzalez-Lopez A. Phase 3 Safety and Efficacy of AZD1222 (ChAdOxI nCoV-19) Covid-19 Vaccine. New England Journal of Medicine. 2021; 385(25):2348-60. doi: 10.1056/NEJMoa2105290.
16. Collier A-rY, Yu J, McMahan K, Liu J, Chandrashekar A, Maron J S, Atyeo C, Martinez D R, Ansel J L, Aguayo R, Rowe M, Jacob-Dolan C, Sellers D, Barrett J, Ahmad K, Anioke T, VanWyk H, Gardner S, Powers O, Bondzie E A, Wan H, Baric R S, Alter G, Hacker M R, Barouch D H. Differential Kinetics of Immune Responses Elicited by Covid-19 Vaccines. New England Journal of Medicine. 2021; 385(21):2010-2. doi: 10.1056/NEJMc2115596.
17. Mahrokhian S H, Tostanoski L H, Jacob-Dolan C, Zahn R C, Wegmann F, McMahan K, Yu J, Gebre M S, Bondzie E A, Wan H, Powers O, Ye T, Barrett J, Schuitemaker H, Barouch D H. Durability and expansion of neutralizing antibody breadth following Ad26.COV2.S vaccination of mice. npj Vaccines. 2022; 7(1):23. doi: 10.1038/s41541-022-00454-4.
18. Barouch D H, Stephenson K E, Sadoff J, Yu J, Chang A, Gebre M, McMahan K, Liu J, Chandrashekar A, Patel S, Le Gars M, de Groot A M, Heerwegh D, Struyf F, Douoguih M, van Hoof J, Schuitemaker H. Durable Humoral and Cellular Immune Responses 8 Months after Ad26.COV2.S Vaccination. New England Journal of Medicine. 2021; 385(10):951-3. doi: 10.1056/NEJMc2108829.
19. Pardi N, Tuyishime S, Muramatsu H, Kariko K, Mui B L, Tam Y K, Madden T D, Hope M J, Weissman D. Expression kinetics of nucleoside-modified mRNA delivered in lipid nanoparticles to mice by various routes. Journal of Controlled Release. 2015; 217:345-51. doi: doi.org/10.1016/j.jconrel.2015.08.007.
20. Geiben-Lynn R, Greenland J R, Frimpong-Boateng K, Letvin N L. Kinetics of recombinant adenovirus type 5, vaccinia virus, modified vaccinia ankara virus, and DNA antigen expression in vivo and the induction of memory T-lymphocyte responses. Clin Vaccine Immunol. 2008; 15(4):691-6. Epub 2008/02/13. doi: 10.1128/CVI.00418-07. PubMed PMID: 18272665.
21. Suthar M S, Zimmerman M G, Kauffman R C, Mantus G, Linderman S L, Hudson W H, Vanderheiden A, Nyhoff L, Davis C W, Adekunle O, Affer M, Sherman M, Reynolds S, Verkerke H P, Alter D N, Guarner J, Bryksin J, Horwath M C, Arthur C M, Saakadze N, Smith G H, Edupuganti S, Scherer E M, Hellmeister K, Cheng A, Morales J A, Neish A S, Stowell S R, Frank F, Ortlund E, Anderson E J, Menachery V D, Rouphael N, Mehta A K, Stephens D S, Ahmed R, Roback J D, Wrammert J. Rapid Generation of Neutralizing Antibody Responses in COVID-19 Patients. Cell Reports Medicine. 2020; 1(3):100040. doi: doi.org/10.1016/j.xcrm.2020.100040.
22. Piccoli L, Park Y-J, Tortorici M A, Czudnochowski N, Walls A C, Beltramello M, Silacci-Fregni C, Pinto D, Rosen L E, Bowen J E, Acton O J, Jaconi S, Guarino B, Minola A, Zatta F, Sprugasci N, Bassi J, Peter A, De Marco A, Nix J C, Mele F, Jovic S, Rodriguez B F, Gupta S V, Jin F, Piumatti G, Lo Presti G, Pellanda A F, Biggiogero M, Tarkowski M, Pizzuto M S, Cameroni E, Havenar-Daughton C, Smithey M, Hong D, Lepori V, Albanese E, Ceschi A, Bernasconi E, Elzi L, Ferrari P, Garzoni C, Riva A, Snell G, Sallusto F, Fink K, Virgin H W, Lanzavecchia A, Corti D, Veesler D. Mapping Neutralizing and Immunodominant Sites on the SARS-CoV-2 Spike Receptor-Binding Domain by Structure-Guided High-Resolution Serology. Cell. 2020; 183(4):1024-42.e21. doi: doi.org/10.1016/j.cell.2020.09.037.
23. Gilbert P B, Montefiori D C, McDermott A B, Fong Y, Benkeser D, Deng W, Zhou H, Houchens C R, Martins K, Jayashankar L, Castellino F, Flach B, Lin B C, O'Connell S, McDanal C, Eaton A, Sarzotti-Kelsoe M, Lu Y, Yu C, Borate B, Laan L WPvd, Hejazi N S, Huynh C, Miller J, Sahly H M E, Baden L R, Baron M, Cruz L D L, Gay C, Kalams S, Kelley C F, Andrasik M P, Kublin J G, Corey L, Neuzil K M, Carpp L N, Pajon R, Follmann D, Donis R O, Koup R A. Immune correlates analysis of the mRNA-1273 COVID-19 vaccine efficacy clinical trial. Science. 2022; 375(6576):43-50. doi: doi:10.1126/science.abm3425.
24. Feng S, Phillips D J, White T, Sayal H, Aley P K, Bibi S, Dold C, Fuskova M, Gilbert S C, Hirsch I, Humphries H E, Jepson B, Kelly E J, Plested E, Shoemaker K, Thomas K M, Vekemans J, Villafana T L, Lambe T, Pollard A J, Voysey M, Adlou S, Allen L, Angus B, Anslow R, Asselin M-C, Baker N, Baker P, Barlow T, Beveridge A, Bewley K R, Brown P, Brunt E, Buttigieg K R, Camara S, Charlton S, Chiplin E, Cicconi P, Clutterbuck E A, Collins A M, Coombes N S, Clemens S A C, Davison M, Demissie T, Dinesh T, Douglas A D, Duncan C J A, Emary K R W, Ewer K J, Felle S, Ferreira D M, Finn A, Folegatti P M, Fothergill R, Fraser S, Garlant H, Gatcombe L, Godwin K J, Goodman A L, Green C A, Hallis B, Hart T C, Heath P T, Hill H, Hill A V S, Jenkin D, Kasanyinga M, Kerridge S, Knight C, Leung S, Libri V, Lillie P J, Marinou S, McGlashan J, McGregor A C, McInroy L, Minassian A M, Mujadidi Y F, Penn E J, Petropoulos C J, Pollock K M, Proud P C, Provstgaard-Morys S, Rajapaska D, Ramasamy M N, Sanders K, Shaik I, Singh N, Smith A, Snape M D, Song R, Shrestha S, Sutherland R K, Thomson E C, Turner D P J, Webb-Bridges A, Wrin T, Williams C J, the Oxford CVTG. Correlates of protection against symptomatic and asymptomatic SARS-CoV-2 infection. Nature Medicine. 2021; 27(11):2032-40. doi: 10.1038/s41591-021-01540-1.
25. Sui Y, Bekele Y, Berzofsky J A. Potential SARS-CoV-2 Immune Correlates of Protection in Infection and Vaccine Immunization. Pathogens. 2021; 10(2):138. PubMed PMID: doi:10.3390/pathogens10020138.
26. Kleanthous H, Silverman J M, Makar K W, Yoon I-K, Jackson N, Vaughn D W. Scientific rationale for developing potent RBD-based vaccines targeting COVID-19. npj Vaccines. 2021; 6(1):128. doi: 10.1038/s41541-021-00393-6.
27. Pollet J, Chen W-H, Versteeg L, Keegan B, Zhan B, Wei J, Liu Z, Lee J, Kundu R, Adhikari R, Poveda C, Villar M J, de Araujo Leao A C, Altieri Rivera J, Momin Z, Gillespie P M, Kimata J T, Strych U, Hotez P J, Bottazzi M E. SARS-CoV-2 RBD219-NiCl: A yeast-expressed SARS-CoV-2 recombinant receptor-binding domain candidate vaccine stimulates virus neutralizing antibodies and T-cell immunity in mice. Human Vaccines & Immunotherapeutics. 2021; 17(8):2356-66. doi: 10.1080/21645515.2021.1901545.
28. Chen W-H, Wei J, Kundu R T, Adhikari R, Liu Z, Lee J, Versteeg L, Poveda C, Keegan B, Villar M J, de Araujo Leao A C, Rivera J A, Gillespie P M, Pollet J, Strych U, Zhan B, Hotez P J, Bottazzi M E. Genetic modification to design a stable yeast-expressed recombinant SARS-CoV-2 receptor binding domain as a COVID-19 vaccine candidate. Biochimica et Biophysica Acta (BBA)—General Subjects. 2021; 1865(6):129893. doi: doi.org/10.1016/j.bbagen.2021.129893.
29. Lee J, Liu Z, Chen W-H, Wei J, Kundu R, Adhikari R, Rivera J A, Gillespie P M, Strych U, Zhan B, Hotez P J, Bottazzi M E. Process development and scale-up optimization of the SARS-CoV-2 receptor binding domain-based vaccine candidate, RBD219-N1C1. Applied Microbiology and Biotechnology. 2021; 105(10):4153-65. doi: 10.1007/s00253-021-11281-3.
30. Dalvie N C, Rodriguez-Aponte S A, Hartwell B L, Tostanoski L H, Biedermann A M, Crowell L E, Kaur K, Kumru O S, Carter L, Yu J, Chang A, McMahan K, Courant T, Lebas C, Lemnios AA, Rodrigues K A, Silva M, Johnston R S, Naranjo C A, Tracey M K, Brady J R, Whittaker C A, Yun D, Brunette N, Wang J Y, Walkey C, Fiala B, Kar S, Porto M, Lok M, Andersen H, Lewis M G, Love K R, Camp D L, Silverman J M, Kleanthous H, Joshi S B, Volkin D B, Dubois P M, Collin N, King N P, Barouch D H, Irvine D J, Love J C. Engineered SARS-CoV-2 receptor binding domain improves manufacturability in yeast and immunogenicity in mice. Proceedings of the National Academy of Sciences. 2021; 118(38):e2106845118. doi: 10.1073/pnas.2106845118.
31. Mulligan M J, Lyke K E, Kitchin N, Absalon J, Gurtman A, Lockhart S, Neuzil K, Raabe V, Bailey R, Swanson K A, Li P, Koury K, Kalina W, Cooper D, Fontes-Garfias C, Shi P-Y, Tureci O, Tompkins K R, Walsh E E, Frenck R, Falsey A R, Dormitzer P R, Gruber W C, Sahin U, Jansen K U. Phase I/II study of COVID-19 RNA vaccine BNT162b1 in adults. Nature. 2020; 586(7830):589-93. doi: 10.1038/s41586-020-2639-4.
32. Walls A C, Fiala B, Schafer A, Wrenn S, Pham M N, Murphy M, Tse L V, Shehata L, O'Connor M A, Chen C, Navarro M J, Miranda M C, Pettie D, Ravichandran R, Kraft J C, Ogohara C, Palser A, Chalk S, Lee E C, Guerriero K, Kepl E, Chow C M, Sydeman C, Hodge E A, Brown B, Fuller J T, Dinnon K H, III, Gralinski L E, Leist S R, Gully K L, Lewis T B, Guttman M, Chu H Y, Lee K K, Fuller D H, Baric R S, Kellam P, Carter L, Pepper M, Sheahan T P, Veesler D, King N P. Elicitation of Potent Neutralizing Antibody Responses by Designed Protein Nanoparticle Vaccines for SARS-CoV-2. Cell. 2020; 183(5):1367-82.e17. doi: 10.1016/j.cell.2020.10.043.
33. Teng I T, Nazzari A F, Choe M, Liu T, de Souza M O, Petrova Y, Tsybovsky Y, Wang S, Zhang B, Artamonov M, Madan B, Huang A, Lopez Acevedo S N, Pan X, Ruckwardt T J, DeKosky B J, Mascola J R, Misasi J, Sullivan N J, Zhou T, Kwong P D. Molecular probes of spike ectodomain and its subdomains for SARS-CoV-2 variants, Alpha through Omicron. bioRxiv. 2021. Epub 2022/01/13. doi: 10.1101/2021.12.29.474491. PubMed PMID: 35018379; PMCID: PMC8750702.
34. Tuekprakhon A, Huo J, Nutalai R, Dijokaite-Guraliuc A, Zhou D, Ginn H M, Selvaraj M, Liu C, Mentzer A J, Supasa P, Duyvesteyn H M E, Das R, Skelly D, Ritter T G, Amini A, Bibi S, Adele S, Johnson S A, Constantinides B, Webster H, Temperton N, Klenerman P, Barnes E, Dunachie S J, Crook D, Pollard A J, Lambe T, Goulder P, Fry E E, Mongkolsapaya J, Ren J, Stuart D I, Screaton G R. Further antibody escape by Omicron BA.4 and BA.5 from vaccine and BA.1 serum. bioRxiv. 2022:2022.05.21.492554. doi: 10.1101/2022.05.21.492554.
35. Greaney A J, Loes A N, Crawford K H D, Starr T N, Malone K_{D}, Chu H Y, Bloom J D. Comprehensive mapping of mutations in the SARS-CoV-2 receptor-binding domain that affect recognition by polyclonal human plasma antibodies. Cell Host & Microbe. 2021; 29(3):463-76.e6. doi: doi.org/10.1016/j.chom.2021.02.003.
36. Corbett K S, Edwards D K, Leist S R, Abiona O M, Boyoglu-Barnum S, Gillespie R A, Himansu S, Schafer A, Ziwawo C T, DiPiazza A T, Dinnon K H, Elbashir S M, Shaw C A, Woods A, Fritch E J, Martinez D R, Bock K W, Minai M, Nagata B M, Hutchinson G B, Wu K, Henry C, Bahl K, Garcia-Dominguez D, Ma L, Renzi I, Kong W-P, Schmidt S D, Wang L, Zhang Y, Phung E, Chang L A, Loomis R J, Altaras N E, Narayanan E, Metkar M, Presnyak V, Liu C, Louder M K, Shi W, Leung K, Yang E S, West A, Gully K L, Stevens L J, Wang N, Wrapp D, Doria-Rose N A, Stewart-Jones G, Bennett H, Alvarado G S, Nason M C, Ruckwardt T J, McLellan J S, Denison M R, Chappell J D, Moore I N, Morabito K M, Mascola J R, Baric R S, Carfi A, Graham B S. SARS-CoV-2 mRNA vaccine design enabled by prototype pathogen preparedness. Nature. 2020; 586(7830):567-71. doi: 10.1038/s41586-020-2622-0.
37. Vogel A B, Kanevsky I, Che Y, Swanson K A, Muik A, Vormehr M, Kranz L M, Walzer K C, Hein S, Guler A. BNT162b vaccines protect rhesus macaques from SARS-CoV-2. Nature. 2021; 592(7853):283-9.
38. De Genst E J, Guilliams T, Wellens J, O'day EM, Waudby C A, Meehan S, Dumoulin M, Hsu S-TD, Cremades N, Verschueren K H. Structure and properties of a complex of α-synuclein and a single-domain camelid antibody. Journal of molecular biology. 2010; 402(2):326-43.
39. Jin J, Hjerrild K A, Silk S E, Brown R E, Labbe G M, Marshall J M, Wright K E, Bezemer S, Clemmensen S B, Biswas S. Accelerating the clinical development of protein-based vaccines for malaria by efficient purification using a four amino acid C-terminal ‘C-tag’. International journal for parasitology. 2017; 47(7):435-46.
40. Ramasamy S, Kolloli A, Kumar R, Husain S, Soteropoulos P, Chang T L, Subbian S. Comprehensive Analysis of Disease Pathology in Immunocompetent and Immunocompromised Hosts following Pulmonary SARS-CoV-2 Infection. Biomedicines. 2022; 10(6). Epub 2022/06/25. doi: 10.3390/biomedicines10061343. PubMed PMID: 35740365; PMCID: PMC9219777.
41. Ravichandran S, Coyle E M, Klenow L, Tang J, Grubbs G, Liu S, Wang T, Golding H, Khurana S. Antibody signature induced by SARS-CoV-2 spike protein immunogens in rabbits. Sci Transl Med. 2020; 12(550). Epub 2020/06/10. doi: 10.1126/scitranslmed.abc3539. PubMed PMID: 32513867; PMCID: PMC7286538.
42. Wu F, Zhao S, Yu B, Chen Y M, Wang W, Song Z G, Hu Y, Tao Z W, Tian J H, Pei Y Y, Yuan M L, Zhang Y L, Dai F H, Liu Y, Wang Q M, Zheng J J, Xu L, Holmes E C, Zhang Y Z. A new coronavirus associated with human respiratory disease in China. Nature. 2020; 579(7798):265-9. Epub 2020/02/06. doi: 10.1038/s41586-020-2008-3. PubMed PMID: 32015508; PMCID: PMC7094943.
43. Venkataraman A, Yang K, Irizarry J, Mackiewicz M, Mita P, Kuang Z, Xue L, Ghosh D, Liu S, Ramos P. A toolbox of immunoprecipitation-grade monoclonal antibodies to human transcription factors. Nature methods. 2018; 15(5):330.
44. Blackshaw S, Venkataraman A, Irizarry J, Yang K, Anderson S, Campbell E, Gatlin C L, Freeman N L, Basavappa R, Stewart R. The NIH Protein Capture Reagents Program (PCRP): a standardized protein affinity reagent toolbox. Nature methods. 2016; 13(10):805-6.
45. Huang Y J, Acton T B, Montelione G T. DisMeta: A Meta Server for Construct Design and Optimization. In: Chen Y W, editor. Structural Genomics: General Applications. Totowa, NJ: Humana Press; 2014. p. 3-16.
46. Xiao R, Anderson S, Aramini J, Belote R, Buchwald W A, Ciccosanti C, Conover K, Everett J K, Hamilton K, Huang Y J, Janjua H, Jiang M, Kornhaber G J, Lee D Y, Locke J Y, Ma L-C, Maglaqui M, Mao L, Mitra S, Patel D, Rossi P, Sahdev S, Sharma S, Shastry R, Swapna G V T, Tong S N, Wang D, Wang H, Zhao L, Montelione G T, Acton T B. The high-throughput protein sample production platform of the Northeast Structural Genomics Consortium. Journal of structural biology. 2010; 172(1):21-33. doi: doi.org/10.1016/j.jsb.2010.07.011.
47. Song W, Gui M, Wang X, Xiang Y. Cryo-EM structure of the SARS coronavirus spike glycoprotein in complex with its host cell receptor ACE2. PLOS Pathogens. 2018; 14(8):e1007236. doi: 10.1371/journal.ppat.1007236.
48. Jin J, Hjerrild K A, Silk S E, Brown R E, Labbe G M, Marshall J M, Wright K E, Bezemer S, Clemmensen S B, Biswas S, Li Y, El-Turabi A, Douglas A D, Hermans P, Detmers F J, de Jongh W A, Higgins M K, Ashfield R, Draper S J. Accelerating the clinical development of protein-based vaccines for malaria by efficient purification using a four amino acid C-terminal ‘C-tag’. International Journal for Parasitology. 2017; 47(7):435-46. doi: doi.org/10.1016/j.ijpara.2016.12.001.
49. Wu K, Choi A, Koch M, Elbashir S, Ma L, Lee D, Woods A, Henry C, Palandjian C, Hill A, Quinones J, Nunna N, O'Connell S, McDermott A B, Falcone S, Narayanan E, Colpitts T, Bennett H, Corbett K S, Seder R, Graham B S, Stewart-Jones G B, Carfi A, Edwards D K. Variant SARS-CoV-2 mRNA vaccines confer broad neutralization as primary or booster series in mice. bioRxiv. 2021:2021.04.13.439482. doi: 10.1101/2021.04.13.439482.
50. Liang J G, Su D, Song T-Z, Zeng Y, Huang W, Wu J, Xu R, Luo P, Yang X, Zhang X, Luo S, Liang Y, Li X, Huang J, Wang Q, Huang X, Xu Q, Luo M, Huang A, Luo D, Zhao C, Yang F, Han J-B, Zheng Y-T, Liang P. S-Trimer, a COVID-19 subunit vaccine candidate, induces protective immunity in nonhuman primates. Nature Communications. 2021; 12(1):1346. doi: 10.1038/s41467-021-21634-1.
51. Doria-Rose N, Suthar M S, Makowski M, O'Connell S, McDermott A B, Flach B, Ledgerwood J E, Mascola J R, Graham B S, Lin B C, O'Dell S, Schmidt S D, Widge A T, Edara V-V, Anderson E J, Lai L, Floyd K, Rouphael N G, Zarnitsyna V, Roberts P C, Makhene M, Buchanan W, Luke C J, Beigel J H, Jackson L A, Neuzil K M, Bennett H, Leav B, Albert J, Kunwar P. Antibody Persistence through 6 Months after the Second Dose of mRNA-1273 Vaccine for Covid-19. New England Journal of Medicine. 2021; 384(23):2259-61. doi: 10.1056/NEJMc2103916. PubMed PMID: 33822494.
52. Suthar M S, Arunachalam P S, Hu M, Reis N, Trisal M, Raeber O, Chinthrajah S, Davis-Gardner M E, Manning K, Mudvari P, Boritz E, Godbole S, Henry A R, Douek D C, Halfmann P, Kawaoka Y, Boyd S D, Davis M M, Zarnitsyna V I, Nadeau K, Pulendran B. Durability of immune responses to the BNT162b2 mRNA vaccine. Med. 2022; 3(1):25-7. doi: doi.org/10.1016/j.medj.2021.12.005.
53. Weeratna R D, Brazolot Millan C L, McCluskie M J, Davis H L. CpG ODN can re-direct the Th bias of established Th2 immune responses in adult and young mice. FEMS Immunology & Medical Microbiology. 2001; 32(1):65-71. doi: 10.1111/j.1574-695X.2001.tb00535.x.
54. Chan J F, Zhang A J, Yuan S, Poon V K, Chan C C, Lee A C, Chan W M, Fan Z, Tsoi H W, Wen L, Liang R, Cao J, Chen Y, Tang K, Luo C, Cai J P, Kok K H, Chu H, Chan K H, Sridhar S, Chen Z, Chen H, To K K, Yuen K Y. Simulation of the Clinical and Pathological Manifestations of Coronavirus Disease 2019 (COVID-19) in a Golden Syrian Hamster Model: Implications for Disease Pathogenesis and Transmissibility. Clin Infect Dis. 2020; 71(9):2428-46. Epub 2020/03/28. doi: 10.1093/cid/ciaa325. PubMed PMID: 32215622; PMCID: PMC7184405.
55. Ying B, Scheaffer S M, Whitener B, Liang C-Y, Dmytrenko O, Mackin S, Wu K, Lee D, Avena L E, Chong Z, Case J B, Ma L, Kim T T_{M}, Sein C E, Woods A, Berrueta D M, Chang G-Y, Stewart-Jones G, Renzi I, Lai Y-T, Malinowski A, Carfi A, Elbashir S M, Edwards D K, Thackray L B, Diamond M S. Boosting with variant-matched or historical mRNA vaccines protects against Omicron infection in mice. Cell. 2022; 185(9):1572-87.el1. doi: doi.org/10.1016/j.cell.2022.03.037.
56. Shang J, Ye G, Shi K, Wan Y, Luo C, Aihara H, Geng Q, Auerbach A, Li F. Structural basis of receptor recognition by SARS-CoV-2. Nature. 2020; 581(7807):221-4. doi: 10.1038/s41586-020-2179-y.
57. Aroul-Selvam R, Hubbard T, Sasidharan R. Domain Insertions in Protein Structures. Journal of Molecular Biology. 2004; 338(4):633-41. doi: doi.org/10.1016/j.jmb.2004.03.039.
58. Pandya C, Brown S, Pieper U, Sali A, Dunaway-Mariano D, Babbitt P C, Xia Y, Allen K N. Consequences of domain insertion on sequence-structure divergence in a superfold. Proceedings of the National Academy of Sciences. 2013; 110(36):E3381-E7. doi: doi:10.1073/pnas.1305519110.
59. Khare S, Azevedo M, Parajuli P, Gokulan K. Conformational Changes of the Receptor Binding Domain of SARS-CoV-2 Spike Protein and Prediction of a B-Cell Antigenic Epitope Using Structural Data. Frontiers in Artificial Intelligence. 2021; 4. doi: 10.3389/frai.2021.630955.
60. Cai Y, Zhang J, Xiao T, Peng H, Sterling S M, Walsh R M, Jr., Rawson S, Rits-Volloch S, Chen B. Distinct conformational states of SARS-CoV-2 spike protein. Science. 2020; 369(6511):1586-92. Epub 2020/07/23. doi: 10.1126/science.abd4251. PubMed PMID: 32694201; PMCID: PMC7464562.
61. He W-t, Musharrafieh R, Song G, Dueker K, Tse L V, Martinez D R, Schafer A, Callaghan S, Yong P, Beutler N, Torres J L, Volk R M, Zhou P, Yuan M, Liu H, Anzanello F, Capozzola T, Parren M, Garcia E, Rawlings S A, Smith D M, Wilson I A, Safonova Y, Ward A B, Rogers T F, Baric R S, Gralinski L E, Burton D R, Andrabi R. Targeted isolation of diverse human protective broadly neutralizing antibodies against SARS-like viruses. Nature Immunology. 2022; 23(6):960-70. doi: 10.1038/s41590-022-01222-1.
62. Greaney A J, Loes A N, Gentles L E, Crawford K H D, Starr T N, Malone K_{D}, Chu H Y, Bloom J D. Antibodies elicited by mRNA-1273 vaccination bind more broadly to the receptor binding domain than do those from SARS-CoV-2 infection. Science Translational Medicine. 2021; 13(600):eabi9915. doi: doi:10.1126/scitranslmed.abi9915.
63. Liu L, Wang P, Nair M S, Yu J, Rapp M, Wang Q, Luo Y, Chan J F W, Sahi V, Figueroa A, Guo X V, Cerutti G, Bimela J, Gorman J, Zhou T, Chen Z, Yuen K-Y, Kwong P D, Sodroski J G, Yin M T, Sheng Z, Huang Y, Shapiro L, Ho D D. Potent neutralizing antibodies against multiple epitopes on SARS-CoV-2 spike. Nature. 2020; 584(7821):450-6. doi: 10.1038/s41586-020-2571-7.
64. Obermeyer F, Jankowiak M, Barkas N, Schaffner S F, Pyle J D, Yurkovetskiy L, Bosso M, Park D J, Babadi M, MacInnis B L, Luban J, Sabeti P C, Lemieux J E. Analysis of 6.4 million SARS-CoV-2 genomes identifies mutations associated with fitness. Science. 2022; 376(6599):1327-32. doi: doi:10.1126/science.abml208.
65. Lazo L, Bequet-Romero M, Lemos G, Musacchio A, Cabrales A, Bruno A J, Ariel Espinosa L, Saloheimo M, Vitikainen M, Hernindez A, Emalfarb M, Tchelet R, Suzarte E, Guillen G. A recombinant SARS-CoV-2 receptor-binding domain expressed in an engineered fungal strain of Thermothelomyces heterothallica induces a functional immune response in mice. Vaccine. 2022; 40(8):1162-9. doi: doi.org/10.1016/j.vaccine.2022.01.007.
66. Israel A, Shenhar Y, Green I, Merzon E, Golan-Cohen A, Schaffer A A, Ruppin E, Vinker S, Magen E. Large-Scale Study of Antibody Titer Decay following BNT162b2 mRNA Vaccine or SARS-CoV-2 Infection. Vaccines. 2022; 10(1):64. PubMed PMID: doi:10.3390/vaccines10010064.
67. Pegu A, O'Connell S E, Schmidt S D, O'Dell S, Talana C A, Lai L, Albert J, Anderson E, Bennett H, Corbett K S, Flach B, Jackson L, Leav B, Ledgerwood J E, Luke C J, Makowski M, Nason M C, Roberts P C, Roederer M, Rebolledo P A, Rostad C A, Rouphael N G, Shi W, Wang L, Widge A T, Yang E S, Beigel J H, Graham B S, Mascola J R, Suthar M S, McDermott A B, Doria-Rose N A, Arega J, Beigel J H, Buchanan W, Elsafy M, Hoang B, Lampley R, Kolhekar A, Koo H, Luke C, Makhene M, Nayak S, Pikaart-Tautges R, Roberts P C, Russell J, Sindall E, Albert J, Kunwar P, Makowski M, Anderson E J, Bechnak A, Bower M, Camacho-Gonzalez A F, Collins M, Drobeniuc A, Edara V V, Edupuganti S, Floyd K, Gibson T, Ackerley C M G, Johnson B, Kamidani S, Kao C, Kelley C, Lai L, Macenczak H, McCullough M P, Peters E, Phadke V K, Rebolledo P A, Rostad C A, Rouphael N, Scherer E, Sherman A, Stephens K, Suthar M S, Teherani M, Traenkner J, Winston J, Yildirim I, Barr L, Benoit J, Carste B, Choe J, Dunstan M, Erolin R, ffitch J, Fields C, Jackson L A, Kiniry E, Lasicka S, Lee S, Nguyen M, Pimienta S, Suyehira J, Witte M, Bennett H, Altaras N E, Carfi A, Hurley M, Leav B, Pajon R, Sun W, Zaks T, Coler R N, Larsen S E, Neuzil K M, Lindesmith L C, Martinez D R, Munt J, Mallory M, Edwards C, Baric R S, Berkowitz N M, Boritz E A, Carlton K, Corbett K S, Costner P, Creanga A, Doria-Rose N A, Douek D C, Flach B, Gaudinski M, Gordon I, Graham B S, Holman L, Ledgerwood J E, Leung K, Lin B C, Louder M K, Mascola J R, McDermott A B, Morabito K M, Novik L, O'Connell S, O'Dell S, Padilla M, Pegu A, Schmidt S D, Shi W, Swanson P A, Talana C A, Wang L, Widge A T, Yang E S, Zhang Y, Chappell J D, Denison M R, Hughes T, Lu X, Pruijssers A J, Stevens L J, Posavad C M, Gale M, Menachery V, Shi P-Y. Durability of mRNA-1273 vaccine& #x2013; induced antibodies against SARS-CoV-2 variants. Science. 2021; 373(6561):1372-7. doi: doi:10.1126/science.abj4176.
68. Bhattacharya D. Instructing durable humoral immunity for COVID-19 and other vaccinable diseases. Immunity. 2022; 55(6):945-64. doi: 10.1016/j.immuni.2022.05.004.
69. Lightman S M, Utley A, Lee K P. Survival of Long-Lived Plasma Cells (LLPC): Piecing Together the Puzzle. Frontiers in immunology. 2019; 10. doi: 10.3389/fimmu.2019.00965.
70. Nguyen D C, Lamothe P A, Woodruff M C, Saini A S, Faliti C E, Sanz I, Lee F E-H. COVID-19 and plasma cells: Is there long-lived protection?*. Immunological Reviews. 2022; 309(1):40-63. doi: doi.org/10.1111/imr.13115.
71. McKee A S, Munks M W, MacLeod M K, Fleenor C J, Van Rooijen N, Kappler J W, Marrack P. Alum induces innate immune responses through macrophage and mast cell sensors, but these sensors are not required for alum to act as an adjuvant for specific immunity. J Immunol. 2009; 183(7):4403-14. Epub 2009/09/08. doi: 10.4049/jimmunol.0900164. PubMed PMID: 19734227; PMCID: PMC2912728.
72. Pulendran B, P S A, O'Hagan D T. Emerging concepts in the science of vaccine adjuvants. Nature reviews Drug discovery. 2021; 20(6):454-75. Epub 2021/04/08. doi: 10.1038/s41573-021-00163-y. PubMed PMID: 33824489; PMCID: PMC8023785 of GSK and is on the scientific advisory board of Medicago.
73. Nanishi E, Borriello F, O'Meara T R, McGrath M E, Saito Y, Haupt R E, Seo H-S, van Haren S D, Cavazzoni C B, Brook B, Barman S, Chen J, Diray-Arce J, Doss-Gollin S, De Leon M, Prevost-Reilly A, Chew K, Menon M, Song K, Xu A Z, Caradonna T_{M}, Feldman J, Hauser B M, Schmidt A G, Sherman A C, Baden L R, Ernst R K, Dillen C, Weston S M, Johnson R M, Hammond H L, Mayer R, Burke A, Bottazzi M E, Hotez P J, Strych U, Chang A, Yu J, Sage P T, Barouch D H, Dhe-Paganon S, Zanoni I, Ozonoff A, Frieman M B, Levy O, Dowling D J. An aluminum hydroxide:CpG adjuvant enhances protection elicited by a SARS-CoV-2 receptor binding domain vaccine in aged mice. Science Translational Medicine. 2022; 14(629):eabj5305. doi: doi:10.1126/scitranslmed.abj5305.
74. Lien C-E, Lin Y-J, Chen C, Lian W-C, Kuo T-Y, Campbell J D, Traquina P, Lin M-Y, Liu L T-C, Chuang Y-S, Ko H-Y, Liao C-C, Chen Y-H, Jan J-T, Ma H-H, Sun C-P, Lin Y-S, Wu P-Y, Wang Y-C, Tao M-H, Lin Y-L. CpG-adjuvanted stable prefusion SARS-CoV-2 spike protein protected hamsters from SARS-CoV-2 challenge. Scientific Reports. 2021; 11(1):8761. doi: 10.1038/s41598-021-88283-8.
75. Tostanoski L H, Wegmann F, Martinot A J, Loos C, McMahan K, Mercado N B, Yu J, Chan C N, Bondoc S, Starke C E, Nekorchuk M, Busman-Sahay K, Piedra-Mora C, Wrijil L M, Ducat S, Custers J, Atyeo C, Fischinger S, Burke J S, Feldman J, Hauser B M, Caradonna T_{M}, Bondzie E A, Dagotto G, Gebre M S, Jacob-Dolan C, Lin Z, Mahrokhian S H, Nampanya F, Nityanandam R, Pessaint L, Porto M, Ali V, Benetiene D, Tevi K, Andersen H, Lewis M G, Schmidt A G, Lauffenburger D A, Alter G, Estes J D, Schuitemaker H, Zahn R, Barouch D H. Ad26 vaccine protects against SARS-CoV-2 severe clinical disease in hamsters. Nature Medicine. 2020; 26(11):1694-700. doi: 10.1038/s41591-020-1070-6.
76. Pulendran B, S. Arunachalam P, O'Hagan D T. Emerging concepts in the science of vaccine adjuvants. Nature Reviews Drug Discovery. 2021; 20(6):454-75. doi: 10.1038/s41573-021-00163-y.
77. Hornung V, Rothenfusser S, Britsch S, Krug A, Jahrsdorfer B, Giese T, Endres S, Hartmann G. Quantitative Expression of Toll-Like Receptor 1-10 mRNA in Cellular Subsets of Human Peripheral Blood Mononuclear Cells and Sensitivity to CpG Oligodeoxynucleotides. The Journal of Immunology. 2002; 168(9):4531-7. doi: 10.4049/jimmunol.168.9.4531.
78. Arunachalam P S, Feng Y, Ashraf U, Hu M, Walls A C, Edara V V, Zarnitsyna V I, Aye P P, Golden N, Miranda M C, Green K W M, Threeton B M, Maness N J, Beddingfield B J, Bohm R P, Scheuermann S E, Goff K, Dufour J, Russell-Lodrigue K, Kepl E, Fiala B, Wrenn S, Ravichandran R, Ellis D, Carter L, Rogers K, Shirreff L M, Ferrell D E, Deb Adhikary N R, Fontenot J, Hammond H L, Frieman M, Grifoni A, Sette A, O'Hagan D T, Van Der Most R, Rappuoli R, Villinger F, Kleanthous H, Rappaport J, Suthar M S, Veesler D, Wang T T, King N P, Pulendran B. Durable protection against the SARS-CoV-2 Omicron variant is induced by an adjuvanted subunit vaccine. Science Translational Medicine. 2022; 14(658):eabg4130. doi: doi:10.1126/scitranslmed.abg4130.
79. Grigoryan L, Lee A, Walls A C, Lai L, Franco B, Arunachalam P S, Feng Y, Luo W, Vanderheiden A, Floyd K, Wrenn S, Pettie D, Miranda M C, Kepl E, Ravichandran R, Sydeman C, Brunette N, Murphy M, Fiala B, Carter L, Coffman R L, Novack D, Kleanthous H, O'Hagan D T, van der Most R, McLellan J S, Suthar M, Veesler D, King N P, Pulendran B. Adjuvanting a subunit SARS-CoV-2 vaccine with clinically relevant adjuvants induces durable protection in mice. npj Vaccines. 2022; 7(1):55. doi: 10.1038/s41541-022-00472-2.
80. Arunachalam P S, Walls A C, Golden N, Atyeo C, Fischinger S, Li C, Aye P, Navarro M J, Lai L, Edara V V, Roltgen K, Rogers K, Shirreff L, Ferrell D E, Wrenn S, Pettie D, Kraft J C, Miranda M C, Kepl E, Sydeman C, Brunette N, Murphy M, Fiala B, Carter L, White A G, Trisal M, Hsieh C-L, Russell-Lodrigue K, Monjure C, Dufour J, Spencer S, Doyle-Meyers L, Bohm R P, Maness N J, Roy C, Plante J A, Plante K S, Zhu A, Gorman M J, Shin S, Shen X, Fontenot J, Gupta S, O'Hagan D T, Van Der Most R, Rappuoli R, Coffman R L, Novack D, McLellan J S, Subramaniam S, Montefiori D, Boyd S D, Flynn J L, Alter G, Villinger F, Kleanthous H, Rappaport J, Suthar M S, King N P, Veesler D, Pulendran B. Adjuvanting a subunit COVID-19 vaccine to induce protective immunity. Nature. 2021; 594(7862):253-8. doi: 10.1038/s41586-021-03530-2.
81. Thuluva S, Paradkar V, Gunneri S R, Yerroju V, Mogulla R, Turaga K, Kyasani M, Manoharan S K, Medigeshi G, Singh J, Shaman H, Singh C, Rao A V. Evaluation of safety and immunogenicity of receptor-binding domain-based COVID-19 vaccine (Corbevax™) to select the optimum formulation in open-label, multicentre, and randomised phase-1/2 and phase-2 clinical trials. eBioMedicine. 2022; 83. doi: 10.1016/j.ebiom.2022.104217.
82. Pollet J, Strych U, Chen W-H, Versteeg L, Keegan B, Zhan B, Wei J, Liu Z, Lee J, Kundu R, Adhikari R, Poveda C, Jose Villar M, Rani Thimmiraju S, Lopez B, Gillespie P M, Ronca S, Kimata J T, Reers M, Paradkar V, Hotez P J, Elena Bottazzi M. Receptor-binding domain recombinant protein on alum-CpG induces broad protection against SARS-CoV-2 variants of concern. Vaccine. 2022; 40(26):3655-63. doi: doi.org/10.1016/j.vaccine.2022.05.007.
83. Su D, Li X, Huang X, He C, Zeng C, Wang Q, Qin W, Mu Z, Ambrosino D, Siber G, Clemens R, Liang J G, Liang P, Jackson N, Xu R. Protection from Omicron and other VOCs by Bivalent S-Trimer COVID-19 Vaccine. bioRxiv. 2022:2022.05.03.490428. doi: 10.1101/2022.05.03.490428.
84. Haidar G, Mellors J W. Improving the Outcomes of Immunocompromised Patients With Coronavirus Disease 2019. Clinical Infectious Diseases. 2021; 73(6):e1397-e401. doi: 10.1093/cid/ciab397.
85. Galmiche S, Luong Nguyen L B, Tartour E, de Lamballerie X, Wittkop L, Loubet P, Launay O. Immunological and clinical efficacy of COVID-19 vaccines in immunocompromised populations: a systematic review. Clinical Microbiology and Infection. 2022; 28(2):163-77. doi: doi.org/10.1016/j.cmi.2021.09.036.
86. Riise J, Meyer S, Blaas I, Chopra A, Tran T T, Delic-Sarac M, Hestdalen M L, Brodin E, Rustad E H, Dai K-Z, Vaage J T, Nissen-Meyer L S H, Sund F, Wader K F, Bjornevik A T, Meyer P A, Nygaard G O, Konig M, Smeland S, Lund-Johansen F, Olweus J, Kolstad A. Rituximab-treated patients with lymphoma develop strong CD8 T-cell responses following COVID-19 vaccination. British Journal of Haematology. 2022; 197(6):697-708. doi: doi.org/10.1111/bjh.18149.
87. Shree T, Shankar V, Lohmeyer J J K, Czerwinski D K, Schroers-Martin J G, Rodriguez G M, Beygi S, Kanegai A M, Corbelli K S, Gabriel E, Kurtz D M, Khodadoust M S, Gupta N K, Maeda L S, Advani R H, Alizadeh A A, Levy R. CD20-Targeted Therapy Ablates De Novo Antibody Response to Vaccination but Spares Preestablished Immunity. Blood Cancer Discov. 2022; 3(2):95-102. Epub 2022/01/12. doi: 10.1158/2643-3230.Bcd-21-0222. PubMed PMID: 35015688.
88. Bowe B, Xie Y, Al-Aly Z. Acute and postacute sequelae associated with SARS-CoV-2 reinfection. Nature Medicine. 2022. doi: 10.1038/s41591-022-02051-3.
89. Gilbert P B, Donis R O, Koup R A, Fong Y, Plotkin S A, Follmann D. A Covid-19 Milestone Attained—A Correlate of Protection for Vaccines. New England Journal of Medicine. 2022; 387(24):2203-6. doi: 10.1056/NEJMp2211314.
90. Krammer F. SARS-CoV-2 vaccines in development. Nature. 2020; 586(7830):516-27. doi: 10.1038/s41586-020-2798-3.
91. Kumar A, Bernasconi V, Manak M, de Almeida Aranha A P, Kristiansen P A. The CEPI centralised laboratory network: supporting COVID-19 vaccine development. The Lancet. 2021; 397(10290):2148-9. doi: 10.1016/S0140-6736(21)00982-X.

1. A fusion polypeptide comprising a SARS-CoV-2 Spike polypeptide fragment comprising at least a portion of the N-terminal domain, domains CD1, RBM, and CD2, and at least a portion of CTD1, wherein the N- or C-terminus of the Spike polypeptide fragment is fused to a heterologous N- or C-terminal tag comprising at least two, at least three, or at least four amino acids.

2. A fusion polypeptide comprising a SARS-CoV-2 Spike polypeptide fragment comprising at least a portion of the N-terminal domain, domains CD1, RBM, and CD2, and at least a portion of each of NT and CTD1, wherein the N- or C-terminus of the Spike polypeptide fragment is fused to a heterologous N- or C-terminal tag comprising at least two, at least three, or at least four amino acids, wherein the N- and C-terminal residues of the Spike polypeptide fragment are comprised within an antiparallel beta-sheet.

3. The fusion polypeptide of claim 1 or 2, wherein the heterologous N- or C-terminal tag comprises a C-terminal tag.

4. The fusion polypeptide of claim 3, wherein the C-terminal tag comprises the amino acid sequence Glu-Pro-Glu-Ala (EPEA (SEQ ID NO: 103)).

5. The fusion polypeptide of claim 3, wherein the C-terminal tag consists of the amino acid sequence Glu-Pro-Glu-Ala (EPEA (SEQ ID NO: 103)).

6. A fusion polypeptide comprising a Spike polypeptide fragment comprising the amino acid sequence of residues 316-594 of SEQ ID NO: 1 or comprising an amino acid sequence of a Spike polypeptide fragment that aligns with residues 316-594 of SEQ ID NO: 1, wherein the N- or C-terminus of the Spike polypeptide fragment is fused to a heterologous N- or C-terminal tag comprising at least two, at least three, or at least four amino acids.

7. The fusion polypeptide of claim 6, wherein the heterologous N- or C-terminal tag comprises a C-terminal tag.

8. The fusion polypeptide of claim 7, wherein the C-terminal tag comprises the amino acid sequence Glu-Pro-Glu-Ala (EPEA (SEQ ID NO: 103)).

9. The fusion polypeptide of claim 7, wherein the C-terminal tag consists of the amino acid sequence Glu-Pro-Glu-Ala (EPEA (SEQ ID NO: 103)).

10. The fusion polypeptide of any one of claims 1-9, wherein the Spike polypeptide fragment comprises an amino acid sequence at least 90%, at least 95%, at least 97%, at least 98%, or at least 99% identical to the amino acid sequence of SEQ ID NO: 3.

11. The fusion polypeptide of any one of claims 1-10, wherein the fusion polypeptide comprises the amino acid sequence of SEQ ID NO: 4.

12. The fusion polypeptide of any one of claims 1-10, wherein the fusion polypeptide consists of the amino acid sequence of SEQ ID NO: 4.

13. The fusion polypeptide of any one of claims 1-10, wherein the fusion polypeptide comprises an amino acid sequence at least 90%, at least 95%, at least 97%, at least 98%, or at least 99% identical to the amino acid sequence of SEQ ID NO: 4.

14. The fusion polypeptide of any one of claims 1-5, wherein the Spike polypeptide fragment comprises an amino acid sequence at least 90%, at least 95%, at least 97%, at least 98%, or at least 99% identical to the amino acid sequence of SEQ ID NO: 5.

15. The fusion polypeptide of any one of claims 1-5, wherein the fusion polypeptide comprises the amino acid sequence of SEQ ID NO: 6.

16. The fusion polypeptide of any one of claims 1-5, wherein the fusion polypeptide consists of the amino acid sequence of SEQ ID NO: 6.

17. The fusion polypeptide of any one of claims 1-5, wherein the fusion polypeptide comprises an amino acid sequence at least 90%, at least 95%, at least 97%, at least 98%, or at least 99% identical to the amino acid sequence of SEQ ID NO: 6.

18. The fusion polypeptide of any one of claims 1-5, wherein the Spike polypeptide fragment comprises an amino acid sequence at least 90%, at least 95%, at least 97%, at least 98%, or at least 99% identical to the amino acid sequence of SEQ ID NO: 77.

19. The fusion polypeptide of any one of claims 1-5, wherein the fusion polypeptide comprises the amino acid sequence of SEQ ID NO: 78.

20. The fusion polypeptide of any one of claims 1-5, wherein the fusion polypeptide consists of the amino acid sequence of SEQ ID NO: 78.

21. The fusion polypeptide of any one of claims 1-5, wherein the fusion polypeptide comprises an amino acid sequence at least 90%, at least 95%, at least 97%, at least 98%, or at least 99% identical to the amino acid sequence of SEQ ID NO: 78.

22. The fusion polypeptide of any one of claims 1-5, wherein the Spike polypeptide fragment comprises an amino acid sequence at least 90%, at least 95%, at least 97%, at least 98%, or at least 99% identical to the amino acid sequence of any one of SEQ ID NOs: 7-76, 79, 81, 83, 85, 87, 89, 91, 93, 95, 97, or 99.

23. The fusion polypeptide of any one of claims 1-5, wherein the fusion polypeptide comprises the amino acid sequence of any one of SEQ ID NOs: 7-100.

24. The fusion polypeptide of any one of claims 1-5, wherein the fusion polypeptide consists of the amino acid sequence of any one of SEQ ID NOs: 7-76 followed at the C-terminus by the amino acid sequence EPEA (SEQ ID NO: 103), or consists of the amino acid sequence of any one of SEQ ID NOs: 80, 82, 84, 86, 88, 90, 92, 94, 96, 98, or 100.

25. The fusion polypeptide of any one of claims 1-5, wherein the fusion polypeptide comprises an amino acid sequence at least 90%, at least 95%, at least 97%, at least 98%, or at least 99% identical to the amino acid sequence of any one of SEQ ID NOs: 7-100.

26. The fusion polypeptide of any one of claims 1-5, 10, 13, 14, 17, 18, 21, 22, 24, or 25, wherein the amino acid sequence comprises one or more of the following substitutions:
(a) a substitution at amino acid position 365 (with reference to SEQ ID NO: 1), such as Y365L;
(b) a substitution at amino acid position 511 (with reference to SEQ ID NO: 1), such as V511A;
(c) a substitution at amino acid position 402 (with reference to SEQ ID NO: 1), such as I402V;
and/or
(d) substitutions at amino acid positions 519-521 (with reference to SEQ ID NO: 1) so as to engineer an N-X-T sequence at those amino acid positions, wherein X is any residue but proline.

27. The fusion polypeptide of any one of claims 1-26, wherein the fusion polypeptide is expressed in a fungal cell, such as a yeast cell, or an animal cell, such as an insect cell, or a mammalian cell, such as an HEK293 cell or CHO cell.

28. A composition comprising a fusion polypeptide comprising two or more of the polypeptides of claim 26(a)-(d), and optionally further comprising the unsubstituted starting fusion polypeptide.

29. A composition comprising a mixture of two or more different fusion polypeptides according to any one of claims 1-27.

30. A pharmaceutical composition comprising the fusion polypeptide of any one of claims 1-27 or the composition of claim 28 or 29 and at least one adjuvant.

31. The pharmaceutical composition of claim 30, wherein the adjuvant comprises an aluminum salt and/or a Toll-like receptor (TLR) agonist.

32. The pharmaceutical composition of claim 31, wherein the TLR agonist is a TLR3, TLR4, TLR7, TLR8, TLR7/8, or TLR9 agonist.

33. The pharmaceutical composition of claim 31, wherein the TLR agonist is a TLR9 agonist.

34. The pharmaceutical composition of claim 33, wherein the TLR9 agonist is a CpG di-nucleotide agonist.

35. The pharmaceutical composition of any one of claims 30-34, wherein the pharmaceutical composition has at least one of the following properties, optionally wherein the adjuvant comprises an aluminum salt and does not comprise a TLR agonist or does not comprise a CpG di-nucleotide agonist:
a. is capable of being administered annually;
b. provokes an immune response in a subject that has a durability of at least 6 months and/or of at least 1 year;
c. provokes an antibody-mediated immune response that does not wane after 6 months and/or after 1 year following administration; and
d. anti-RBD IgG antibody titer in a blood sample from a subject administered the pharmaceutical composition does not significantly reduce after 6 months, and/or after 1 year following administration.

36. A polynucleotide molecule that encodes the fusion polypeptide of any one of claims 1-26.

37. The polynucleotide molecule of claim 36, wherein the polynucleotide molecule is a viral vector.

38. A host cell that expresses the polynucleotide molecule of claim 36 or the vector of claim 37.

39. A method of preparing the fusion polypeptide of any one of claims 1-26, comprising incubating the host cell of claim 38 under conditions allowing for expression of the fusion polypeptide, and optionally isolating the fusion polypeptide expressed by the host cell.

40. A method of vaccinating an individual, comprising administering the fusion polypeptide of any one of claims 1-27 or the pharmaceutical composition of any one of claims 30-35 to the individual.

41. The method of claim 40, wherein the method comprises administering the fusion polypeptide or pharmaceutical composition in a single dose.

42. The method of any one of claims 40-41, wherein the method comprises administering the fusion polypeptide or pharmaceutical composition in two doses within a two- to eight-week period of time.

43. The method of any one of claims 40-42, wherein the fusion polypeptide or pharmaceutical composition is administered to the individual every 6 months, every 9 months, or annually.

44. The method of any one of claims 40-43, wherein administration of the fusion protein (a) provokes an immune response in a subject that has a durability of at least 6 months and/or of at least 1 year; (b) provokes an antibody-mediated immune response that does not wane after 6 months and/or after 1 year following administration; and/or (c) provokes an anti-RBD IgG antibody titer in a blood sample from the subject that does not significantly reduce after 6 months, and/or after 1 year following administration.

45. The method of any one of claims 40-44, wherein the method comprises administering the fusion polypeptide of any one of claims 1-27 concurrently or sequentially with at least one adjuvant.

46. The method of claim 45, wherein the at least one adjuvant comprises an aluminum salt and/or a Toll-like receptor (TLR) agonist.

47. The method of claim 46, wherein the TLR agonist is a TLR3, TLR4, TLR7, TLR8, TLR7/8, or TLR9 agonist.

48. The method of claim 46, wherein the TLR agonist is a TLR9 agonist.

49. The method of claim 48, wherein the TLR9 agonist is a CpG di-nucleotide agonist.

50. The method of claim 46, wherein the at least one adjuvant comprises an aluminum salt.

51. The method of claim 46, wherein the at least one adjuvant does not comprise a TLR agonist or does not comprise a CpG di-nucleotide agonist.

52. A method of obtaining antibodies against a SARS-CoV-2 Spike polypeptide, comprising administering the fusion polypeptide of any one of claims 1-27, the pharmaceutical composition of any one of claims 30-35, or the polynucleotide of claim 36 to an animal, and optionally isolating antibodies produced by the animal.

53. The method of claim 52, wherein antibodies are isolated 2 months, 3 months, 6 months, 9 months, or 12 months following administration of the fusion polypeptide, pharmaceutical composition, or polynucleotide to the animal.

54. An isolated antibody produced by the method of claim 52 or 53.
